(19) United States
(12) Patent Application Publication
(10) Pub. No.: US 2018/0028566 A1
(43) Pub. Date: Feb. 1, 2018

(54) GAMMA DELTA T CELLS AS A TARGET FOR TREATMENT OF SOLID TUMORS

(71) Applicant: New York University, New York, NY (US)

(72) Inventors: George Miller, Englewood, NJ (US); Donnele Daley, New York, NY (US); Constantinos Zambirinis, New York, NY (US)

(21) Appl. No.: 15/664,618

(22) Filed: Jul. 31, 2017

Related U.S. Application Data
(60) Provisional application No. 62/368,453, filed on Jul. 29, 2016.
(60) Provisional application No. 62/507,495, filed on May 17, 2017.

(51) Int. Cl. A61K 35/17; C12N 5/0783; A61K 39/00

(52) U.S. Cl. CPC A61K 35/17; A61K 39/0011; C12N 5/0636; A61K 2039/5158; C12N 2502/99; C12N 2502/11; C12N 2501/515

(57) ABSTRACT

Provided herein are γδ T cell suppressors and methods of using such in detecting and treating solid tumors, as well as detection of solid tumors such as PDA or CRC based on the level of γδ T cells.

### CROSS-REFERENCE TO RELATED APPLICATIONS

This application claims priority to U.S. Provisional Patent Application No. 62/368,453, filed on Jul. 29, 2016, and U.S. Provisional Patent Application No. 62/507,495, filed on May 17, 2017, the disclsoures of which are incorporated herein by reference.

### FEDERALLY SPONSORED RESEARCH

This invention was made with government support under Grant Nos. CA-155649, CA-168611, and CA-193111, awarded by the National Institutes of Health (NIH), and under Grant Nos. P30CA016087 and UL1 TR000038, awarded by the National Center for Advancing Translational Sciences (NCATS). The Government has certain rights in the invention.

### BACKGROUND OF THE DISCLOSURE

Pancreatic ductal adenocarcinoma (PDA) is a devastating disease in which the mortality rate approaches the incidence rate (Yadav and Lowenfels, 2013, Gastroenterology 144, 1252-1261). PDA is almost invariably associated with a robust inflammatory cell infiltrate which has considerable influence on disease progression (Andren-Sandberg et al., 1997, Scand J Gastroenterol 32, 97-103; Clark et al., 2007, Cancer research 67, 9518-9527) Peri-pancreatic leukocytic subsets can have divergent effects on tumorigenesis by either combating cancer growth via antigen-restricted tumoricidal immune responses or by promoting tumor progression via induction of immune suppression (Zheng et al., 2013, Gastroenterology 144, 1230-1240). For example, CD8⁺ T cells and Th1-polarized CD4⁺ T cells mediate tumor-protection in murine models of PDA and are associated with prolonged survival in human disease (De Monte et al., 2011, J Exp Med 208, 469-478; Fukunaga et al., 2004, Pancreas 28, e26-31). Negating cytotoxic CD8⁺ anti-tumor responses by myeloid-derived suppressor cells (MDSC) markedly accelerates PDA growth (Pylayeva-Gupta et al., 2012, Cancer Cell 21, 836-847). Conversely, antigen-restricted Th2-deviated CD4⁺ T cells strongly promote PDA progression in mice (Ochi et al., 2012c, J Exp Med 209, 1671-1687). Accordingly, intra-tumoral CD4⁺ Th2 cell infiltrates correlate with reduced survival in human PDA (De Monte et al., 2011, J Exp Med 208, 469-478; Fukunaga et al., 2004, Pancreas 28, e26-31). Nevertheless, intra-pancreatic γδT cells have not been well characterized and their role remains unclear.

### SUMMARY OF THE DISCLOSURE

This disclosure is based at least in part on the findings that immunosuppressive γδT cells with a uniquely activated phenotype infiltrates the pre-neoplastic pancreas and invasive PDA in a mouse PDA model; deletion of the intra-pancreatic γδT cells markedly protects against oncogenesis in vivo and results in an influx and reactivation of immunogenic Th1 cells and CD8⁺ T cells to the tumor microenvironment (TME).

Accordingly, one aspect of the present disclosure features a method for treating a solid tumor, comprising administering to a subject in need thereof an effective amount of a γδ T cell suppressor. In some embodiments, the γδ T cell suppressor is an agent that inhibits an immunosuppressive γδ T cell, for example, a circulating γδ T cell or a γδ T cell infiltrated into tumor tissue or tumor resident organ in the subject. Such a γδ T cell suppressor may be an antibody that specifically binds a γδ T cell, e.g., a γδ T cell comprising a specific gamma or delta chain, such as a δ1 subunit or δ2 subunit. In some instances, the γδ T cell-binding antibody can be a bi-specific antibody that further binds an αβ T cell or NK cell. In addition, the γδ T cell-binding antibody may be tri-specific, i.e., capable of binding to the γ chain of the γδ T cell, the δ chain of the γδ T cell, and an αβ T cell or NK cell. Alternatively, the γδ T cell suppressor is an antibody that blocks recruitment of immunosuppressive γδ T cell to a tumor site in the subject. Such antibodies include, but are not limited to, antibodies specifically binds CCR2, CCL2, or CCR6. Any of the antibodies described herein may be a human antibody or a humanized antibody.

In other embodiments, the γδ T cell suppressor can be an agent that blocks antigenic expansion of immunosuppressive γδ T cells. Alternatively, the γδ T cell suppressor may be an immune cell (e.g., a T cell or an NK cell) expressing a chimeric receptor that targets immunosuppressive γδ T cells.

The subject to be treated by any of the methods described herein may be a human patient having the solid tumor. Examples include, but are not limited to, pancreatic ductal adenocarincoma (PDA), colorectal cancer (CRC), melanoma, breast cancer, lung cancer (for example, non-small cell lung cancer, NSCLC, and small cell lung cancer, SCLC), upper and lower gastrointestinal malignancies (including, but not limited to, esophageal, gastric, and hepatobiliary cancer), squamous cell head and neck cancer, genitourinary, and sarcomas. The subject may have undergone another anti-tumor therapy, e.g., chemotherapy, radiotherapy, immunotherapy, therapy involving a small molecule kinase inhibitor, surgery, or a combination thereof.

In some examples, the method described herein may further comprise performing another anti-tumor therapy, e.g., those described herein, to the subject. For example, the performing step may comprise administering to the subject an inhibitor of a checkpoint molecule (e.g., PD-1, PD-L1, PD-L2, CTLA-4, LAG3, TIM-3 and A2aR), an agonist of a co-stimulatory receptor (e.g., OX40, GITR, CD137, CD40, CD27, and ICOS), or an inhibitor of an innate immune cell target (e.g., KIR, NKG2A, CD96, TLR, and IDO).

In one example, the subject is administered an inhibitor of a checkpoint molecule, which is an anti-PD-L1 antibody. In another example, the subject is further administered a chemotherapeutic agent, such as gemcitabine or abraxane, or a combination thereof (e.g., folinic acid, fluorouracil, oxaliplatin, and irinotecan, a.k.a., FOLFOXIRI).

In another aspect, described herein is a kit for treating a solid tumor in a subject, the kit comprising: (i) a first pharmaceutical composition that comprises a γδ T cell suppressor, and (ii) a second pharmaceutical composition that comprises a chemotherapeutic agent, an inhibitor of a checkpoint molecule, an agonist of a co-stimulatory receptor, or an inhibitor of an innate immune cell target. Further, the present disclosure provides a pharmaceutical composition, comprising (i) a γδ T cell suppressor, and (ii) an inhibitor of a checkpoint molecule, an agonist of a co-stimulatory receptor, or an inhibitor of an innate immune cell target. Also with the scope of the present disclosure are any of the kits or pharmaceutical compositions described herein for use in treating a solid tumor such as PDA, or for manufacturing a medicament for use in treating the solid tumor.

In yet another aspect, provided herein is a method for analyzing a sample, the method comprising: (i) obtaining a biological sample from a subject (e.g., a human patient) suspected of having a solid tumor, for example, pancreatic ductal adenocarcinoma (PDA) or colorectal cancer (CRC); and (ii) measuring the level of γδ T cells in the biological sample. In some embodiments, the γδ T cells are effector memory γδ T (TEM) cells. Optionally, the method may further comprise measuring the level of a checkpoint molecule (e.g., PD-L1), the level of Galectin-9, or both in the biological sample. Alternatively or in addition, the analysis method may further comprise identifying the subject as having or at risk for a solid tumor, such as PDA or CRC, based on the level of the γδ T cells in the biological sample determined in (ii), wherein an elevated level of γδ T cells relative to that of a control subject is indicative of presence or risk of PDA or CRC. In some embodiments, the method may further comprise performing a treatment of PDA to the subject, if the subject is identified as having or at risk for PDA or CRC.

The biological sample may be a peripheral blood sample, or a tissue sample obtained from a suspected tumor site.

In some embodiments, the measuring step in any of the analysis methods described herein may involve an antibody that specifically binds γδ T cells, for example, an antibody specifically binds γδ T cells expressing a T cell receptor comprising a δ1 subunit, or an antibody specifically binds γδ T cells expressing a T cell receptor comprising a δ2 subunit.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1. γδT cells are ubiquitous and activated in human PDA. (a) Frozen sections of human PDA and normal pancreas were stained using a mAb specific for TCRγ/δ or isotype control. Representative images and quantitative data are shown. (b) Single cell suspensions from human PDA tumors and PBMC were co-stained for CD45, CD3, and TCRγ/δ. The percentage of γδT cells among CD3⁺ cells was calculated. Representative contour plots and summary data are shown. Each dot represents a different patient sample. (c) The percentage of PDA-infiltrating γδT cells among CD45⁺ cells was compared with tumor-infiltrating cells expressing select myeloid differentiation markers. (d) The percentage of PDA-infiltrating and PBMC γδT cells among CD3⁺ cells was compared with that of CD4⁺ and CD8⁺αβT cell subsets in each respective compartment. (e) PBMC and PDA-infiltrating CD3⁺TCRγ/δ⁺ cells from PDA patients were gated and co-stained using mAbs specific for CD45RA and CD27. The gating paradigms for Tnaive, TCM, TEM, and TEM-RA populations are shown. Representative contour plots and quantitative data indicating the fraction of TEM γδT cells in each compartment are indicated. (f) PDA-infiltrating and PBMC γδT cells from PDA patients were stained using mAbs specific for CD62L and (g) Vγ9. Representative histograms and quantitative data are shown. Human data are based on tumor tissue or PBMC analyzed from 9-13 PDA patients (*p<0.05, **p<0.01, ***p<0.001).

FIG. 2. γδT cells are highly prevalent and exhibit a uniquely activated phenotype in murine invasive PDA. (a) C57BL/6-Trdc^{tm1Mal} mice whose γδT cells express GFP were orthotopically implanted with KPC-derived tumor and imaged by intra-vital two-photon laser-scanning microscopy at 21 days. (b) WT mice were orthotopically implanted with KPC-derived tumor cells. On day 21, single cell suspensions of digested PDA tumors and splenocytes were co-stained for CD45, CD3, TCRγ/δ, CD4, and CD8 and analyzed by flow cytometry. Representative contour plots and quantitative data are shown. For the bar graphs, for each set of CD4⁺, CD8⁺ and TCRγ/δ⁺, the bars from left to right are: Spleen, and PDA. (c) WT mice were orthotopically implanted with KPC-derived tumor cells. On day 21 spleen (blue histograms) and PDA-infiltrating (red histograms) γδT cells were gated and tested for co-expression of select surface activation markers and Vγ chains. Representative histogram overlays and summary data from 5 mice are shown. For the bar graphs, for each set of bars for FasL, NK1.1, CD39, CD44, JAML, OX40, Vγ4, and Vγ1, the bars from the left to right are: Spleen γδT cells (blue), and PDA γδT cells (red) (d) Spleen and PDA-infiltrating γδT cells from the same mice were tested for expression of IL-10, (e) IL-17, (f) NKG2D, (g) TLR4, TLR7, TLR9, and (h) CCR2, CCR5, and CCR6. Each experiment was repeated at least 3 times using 3-5 mice per data point (*p<0.05, **p<0.01). For (g) and (h), for each bar set, the bars from left to right are: Spleen, and PDA.

FIG. 3. Ablation of γδT cells protects against pancreatic oncogenesis in a slowly progressive model of PDA. (a) KC;Tcrδ^{+/+} and KC;Tcrδ^{-/-} mice were sacrificed at 3, 6, or 9 months of life (n=10-12 mice/cohort). Representative H&E-stained frozen sections are shown. The percentage of pancreatic area occupied by intact acinar structures, and the fractions of ductal structures exhibiting normal morphology, ADM, or graded PanIN I-III lesions were calculated. (b) Weights of pancreata were compared in 3 month-old KC;Tcrδ^{+/+} and KC;Tcrδ^{-/-} mice. (c) Pancreata from 9 month-old KC;Tcrδ^{+/+} and KC;Tcrδ^{-/-} mice were assayed for peri-tumoral fibrosis using trichrome staining. (d) Kaplan-Meier survival analysis was performed for KC;Tcrδ^{+/+} (n=29) and KC;Tcrδ^{-/-} (n=44) mice (p<0.0001). (e, f) KC;Tcrδ^{+/+} mice were treated with UC3-10A6 or isotype control for 8 weeks beginning at 6 weeks of life. (e) Representative H&E stained pancreatic sections are shown. The percentage of pancreatic area occupied by intact acinar structures, and the fractions of ductal structures exhibiting normal morphology, ADM, or graded PanIN I-III lesions were calculated. (f) Tumor weight was recorded (n=5/group; *p<0.05, **p<0.01).

FIG. 4. γδT cell deletion results in massive CD4⁺ and CD8⁺ T cell infiltration and activation in invasive PDA. (a, b) WT and Tcrδ^{-/-} mice were implanted with KPC-derived tumor cells. On day 21 mice were sacrificed. Frozen pancreatic sections were tested for (a) CD8⁺ and (b) CD4⁺ T cell infiltration by IHC (n=5/group). (c) CD8⁺ T cells infiltrating orthotopically-implanted KPC-derived tumors in WT and Tcrδ^{-/-} mice were tested for expression of CD44, (d) ICOS, (e) CTLA-4, and (f) Granzyme B. (g) Similarly, CD4⁺ T cells infiltrating orthotopically-implanted KPC tumors in WT and Tcrδ^{-/-} mice were tested for expression of CD44, (h) OX40, (i) PD-1, and (j) CD62L. Experiments were repeated more than 3 times with similar results using 5 mice per group (*p<0.05, **p<0.01, ***p<0.001).

FIG. 5. γδT cell deletion results in CD4⁺ T cell Th1 differentiation, CD8⁺ T cell activation, and αβT cell-dependent tumor protection in invasive PDA. (a-d) WT and Tcrδ^{-/-} mice were orthotopically implanted with KPC-derived tumor cells. On day 21, tumor-infiltrating CD4⁺ and CD8⁺ T cells were interrogated for (a) co-expression of TNF-α and IFN-γ, (b) expression of T-bet, (c) GATA-3, and (d) FoxP3. Representative contour plots and quantitative data are shown. Experiments were repeated twice with similar results (n=5/group; *p<0.05). (e) WT and Tcrδ^{-/-} pancreata were orthotopically implanted with KPC-derived tumor cells and serially treated with α-CD4 and α-CD8 neutralizing mAbs or isotype controls. Pancreatic tumors were harvested at 3 weeks. Representative images and tumor weights are shown (n=5/group; *p<0.05, **p<0.01, ***p<0.001).

FIG. 6. PDA-associated γδT cells express high levels of T cell exhaustion ligands in multiple murine tumor models and in human disease. (a) Expression of PD-L1 and (b) Galectin-9 were compared in pancreas and spleen γδT cells of 3-month-old KC mice by flow cytometry. Representative contour plots and quantitative data are shown (n=5/group). (c) WT mice were orthotopically implanted with KPC-derived tumor cells. Expression of PD-L1 and Galectin-9 were compared in PDA tumor cells, TAMs (Mφ), MDSC, and γδT cells on day 21 (n=5/group). For each set of bars for Tumor, Mφ, MDSC, and TCRγ6, the bars from left to right are: PD-L1, and Galectin-9 (d) WT mice were orthotopically implanted with KPC-derived tumor cells. On day 21, spleen and PDA-infiltrating γδT cells were tested for expression of select activating ligands. Representative histograms and quantitative data are shown (n=5/group). For each set of bars for B7-1, B7-2, ICOSL, and Ox40L, the bars from left to right are: Spleen, and PDA (e) Orthotopic PDA-bearing WT and Tcrδ^{-/-} mice were tested for expression of PD-L1 in tumor cells, TAMs, and MDSC (n=5/group). For each set of bars for Tumor, Mφ, and MDSC, the bars from left to right are: WT, and TCRγ^{-/-} (f) WT, CCR2^{-/-}, CCR5^{-/-}, and CCR6^{-/-} mice were orthotopically implanted with KPC-derived PDA cells (n=5/group). Animals were sacrificed at 3 weeks, and the fraction of tumor-infiltrating γδT cells expressing PD-L1 and (g) Galectin-9 were determined by flow cytometry. (h, i) PBMC γδT cells from healthy volunteers and PDA patients, and PDA-infiltrating γδT cells and were tested for expression of (h) PD-L1 and (i) Galectin-9. Representative histograms and quantitative data are shown (n=11 patients; *p<0.05, **p<0.01, ***p<0.001).

FIG. 7. Exhaustion ligand blockade reverses the direct suppressive effects of γδT cells on αβT cells and on pancreatic tumorigenesis. (a) Splenic CD4⁺ or (b) CD8⁺ T cells from untreated WT mice were either unstimulated, or stimulated with αCD3/αCD28 alone or in co-culture with PDA-infiltrating γδT cells (5:1 ratio). αPD-L1 (10 μg/ml) was selectively added to each group. The fraction of CD62L⁻ CD44⁺ cells were determined at 72 h by flow cytometry. Representative contour plots and quantitative data are shown. (c) Similarly, CD4⁺ and CD8⁺ T cell expression of TNF-α was measured. Experiments were performed in quadruplicate and repeated 3 times. (d) WT and Tcrδ^{-/-} mice were orthotopically implanted with KPC-derived tumor cells and serially treated with αPD-L1 or αGalectin-9 neutralizing mAbs, or respective isotype controls. Pancreatic tumors were harvested at 3 weeks. Representative gross images are shown (Experiment #1) as are quantitative data on tumor weights from 2 separate experiments using different stocks of KPC-derived tumor cells (n=5/group for each experiment). (e-g) WT and Tcrδ^{-/-} pancreata were again orthotopically implanted with KPC-derived tumor cells and serially treated with αPD-L1 or αGalectin-9 neutralizing mAbs or the respective isotype controls. Pancreatic tumors were harvested at 3 weeks. (e) The fraction of PDA-infiltrating αβT cells among CD45⁺ leukocytes, and (f) CD8⁺ and (g) CD4⁺ T cell adoption of an activated CD62L⁻ CD44⁺ phenotype, were determined by flow cytometry (n=5/group; *p<0.05, **p<0.01, ***p<0.01).

FIG. 8. PDA-infiltrating γδT cells express elevated FoxP3. (a) WT mice were orthotopically implanted with KPC-derived tumor cells. On day 21, splenic and PDA-infiltrating CD3⁺ T lymphocytes were co-stained for CD4, TCRγ6, and FoxP3 or (b) CD4, TCRγ6, and T-bet. Representative contour plots and quantitative data from 5 mice per group are shown (*p<0.05). Experiments were repeated twice with similar results.

FIG. 9. γδT cells in pancreata of KC mice exhibit a distinct phenotype. (a) Single cell suspensions of pancreata, pancreas-draining lymph nodes, and spleen, from 6 month-old KC mice were co-stained for CD45, CD3, and TCRγ/δ. Representative contour plots and quantitative data are shown. For each set of bars for CD4⁺, CD8⁺, and TCR^{γ/δ+}, the bars from left to right are: Spleen, Lymph Node, and PDA, (b) Similarly, select CCR expression. For each set of bars for CCR2, CCR5, and CCR6, the bars from left to right are: Spleen γδT cells, and PDA γ/δT cells, (c) TLR expression. For each set of bars for TLR4, TLR7, and TLR9, the bars from left to right are: Spleen γδT cells, and PDA γδT cells, (d) and surface markers were compared in γδT cells harvested from the pancreas and spleen of 6 month-old KC mice. For each set of bars for NK1.1, CD39, JAML, OX40, and V γ 4, the bars from left to right are: Spleen γδT cells, and PDA γδT cells. Each result was reproduced at least twice (n=5/group; *p<0.05, **p<0.01, ***p<0.001, ****p<0.0001).

FIG. 10. Selective blockade of chemokine signaling mitigates γδT cell expansion and activation in PDA and deletion or depletion of γδT cells or interruption of their recruitment is protective against PDA. (a-e) WT, CCR2^{-/-}, CCL2^{-/-}, CCR5^{-/-}, and CCR6^{-/-} mice were orthotopically implanted with KPC-derived PDA cells (n=5/group). Animals were sacrificed at 3 weeks. (a) The fraction of tumor-infiltrating γδT cells determined by flow cytometry. (b) γδT cell expression of TNF-α, (c) IL-13, (d) IL-17, and (e) IFN-γ was determined for each cohort (n=5/group). (f) WT mice were treated with UC3-10A6 and splenocytes from these mice were analyzed for expression of Vγ4 and Vγ1. (g) WT and Tcrδ^{-/-} pancreata were orthotopically implanted with KPC-derived PDA cells. Tumors were harvested and weighed at 3 weeks after implantation. Representative gross images of PDA and quantitative data of tumor weights are shown (n=10/group). (h) Pancreata from control WT mice, WT mice treated with a neutralizing α-γδT cell mAb, and Tcrδ^{-/-} mice were orthotopically implanted with KPC-derived PDA cells. Kaplan-Meier survival analysis was performed (n=10/group; WT vs Tcrδ^{-/-}: p=0.02; WT vs UC3-10A6: p=0.009; Tcrδ^{-/-} vs UC3-10A6: p=ns). (i) WT, CCR5^{-/-}, CCR6^{-/-}, CCR2^{-/-}, and CCL2^{-/-} mice were orthotopically implanted with KPC-derived PDA cells. Animals were sacrificed at 3 weeks and tumor weights were recorded. Data from 2 separate experiments are shown (n=5/group for each experiment; scale bar=2 cm; *p<0.05, **p<0.01, ****p<0.0001).

FIG. 11. γδT cells do not directly modulate pancreatitis or transformed epithelial cells. (a) Acute pancreatitis was induced using caerulein in C57BL/6-Trdc^{tm1Mal} mice, which express GFP exclusively in γδT cells. Pancreata were harvested at 12 h and immunohostochemistry for GFP was performed. Arrows indicate GFP⁺ cells. (b) Pancreata and spleens of WT mice undergoing caerulein-induced pancreatitis were assessed by flow cytometry for the presence of CD3⁺ TCRγ/δ⁺ cells. The percentage of γδT cells among the intra-pancreatic or spleen T lymphocyte populations, respectively, was calculated at 48 h after commencing caerulein treatment (n=5/group; ****p<0.0001). (c) WT mice were administered caerulein for up to 48 h and then serially observed for a maximum additional 48 h. Cohorts were sacrificed at 0 (untreated), 24, 48, 72, or 96 hours from commencing caerulein treatment and the percentage of pancreas-infiltrating CD4⁺ T cells and γδT cells among CD3⁺ T cells was assessed by flow cytometry (n=3 mice/time-point). (d-g) WT and Tcrδ^{-/-} mice were induced to develop caerulein pancreatitis for 48 h. (d) Severity of pancreatitis was assessed by H&E staining, (e) CD45⁺ pan-leukocyte IHC, and (f) serum amylase and (g) lipase levels (n=5/group). Pancreatitis experiments were repeated more than 5 times with similar results. Representative H&E- and CD45-stained sections are shown. (h-j) KPC-derived PDA cells were co-cultured with PDA-infiltrating or spleen γδT cells in a 1:5 ratio for 24 h. (h) Tumor cell proliferation was measured using the XTT assay. (i) Expression of tumor suppressor or oncogenic proteins was assessed by Western blotting. (j) Expression of tumor-modulatory cytokines was determined in a cytometric bead array. In (j), for each set of bars for IL-6, IL-10, and TNF-α, the bars from left to right are: Control, Splenic γδT cells, and PDA γδT cells. Co-culture experiments were repeated 3 times with similar findings.

FIG. 12. γδT cell deletion results in marked αβT cell expansion and activation in a slowly progressive model of PDA. Cohorts of KC;Tcrδ^{+/+} and KC;Tcrδ^{-/-} mice were sacrificed at 3 months of life. Frozen pancreatic sections were tested for (a) CD4⁺ and (b) CD8+ T cell infiltration by immunohistochemistry (n=8/group). (c) Pancreas draining lymph nodes from 3 month old KC;Tcrδ^{+/+} and KC;Tcrδ^{-/-} mice were harvested and tested for CD4⁺ and CD8⁺ T cell expression of CD44. For each set of bars for CD4⁺ and CD8⁺, the bars from left to right are: KC;Tcrδ^{+/+} and KC;Tcrδ^{-/-}, and (d) PD-1. For each set of bars for CD4⁺ and CD8⁺, the bars from left to right are: KC;Tcrδ^{+/+} and KC;Tcrδ^{-/-}, (e) CD8⁺ T cell expression of ICOS and Granzyme B. For each set of bars for ICOS and Granzyme B, the bars from left to right are: KC;Tcrδ^{+/+} and KC;Tcrδ^{-/-}, (f) CD4⁺ and CD8⁺ T cell expression of IFN-γ. For each set of bars for CD4⁺ and CD8⁺, the bars from left to right are: KC;Tcrδ^{+/+} and KC;Tcrδ^{-/-}, and (g) T-bet. For each set of bars for CD4⁺ and CD8⁺, the bars from left to right are: KC;Tcrδ^{+/+} and KC;Tcrδ^{-/-}, and (h) CD4+ T cell expression of GATA-3 and (i) FoxP3. Representative contour plots and quantitative data are shown. Experiments were repeated 2-3 times with similar results (n=5/group; *p<0.05, ****p<0.0001).

FIG. 13. PDA-infiltrating γδT cells inhibit αβT cells but exhaustion ligand blockade is tumor-protective and activates CD8+ T cells in γδT cell-competent hosts. (a-e) Splenic αβT cells from untreated WT mice were cultured in 96 well plates either unstimulated, or stimulated with αCD3/αCD28 alone or in co-culture with PDA-infiltrating γδT cells (5:1 ratio) or γδT cell conditioned media. The fraction of CD4⁺ and CD8⁺ T cells (a, b) adopting a CD62L⁻ CD44⁺ phenotype and (c, d) expressing IFN-γ was determined at 72 h by flow cytometry. (e) Cytokine expression was also determined by analysis of cell culture supernatant. Experiments were repeated twice (n=4/group). For each set of bars for IFN-γ, TNF-α, IL-4, and IL-10, the bars from left to right are: Unstim αβT cells, αβT cells+Control Sup., αβT cells+γδT cells, and αβT cells+γδT Sup.(f) WT mice were orthotopically implanted with KPC-derived tumor cells and select cohorts were serially treated with an αGalectin-9 neutralizing mAb. Survival was measured according to the Kaplan-Meier method (n=10 mice/group). (g, h) WT and Tcrδ^{-/-} mice were orthotopically implanted with KPC-derived tumor cells and serially treated with αPD-L1 or αGalectin-9 neutralizing mAbs or respective isotype controls. Pancreatic tumors were harvested at 3 weeks, and CD8⁺ T lymphocytes from each cohort were analyzed by flow cytometry for expression of (g) T-bet and (h) TNF-α. (i) Cohorts of 6 week old KC;TCRδ^{+/+} and KC;TCRδ^{-/-} mice were serially treated with αPD-L1 or isotype control for 8 weeks and pancreata were harvested at 14 weeks. Comparative tumor weights are shown (n=5/group). (j, k) Cohorts of WT mice were orthotopically implanted with KPC-derived tumor cells. In parallel, Tcrδ^{-/-} mice were similarly treated but tumor cells were co-injected with FACS-sorted PDA-infiltrating γδT cells that were treated ex-vivo with either (j) Rat IgG isotype, or (k) αPD-L1. PDA tumors were measured at 21 days (n=4/group). (l-m) Tcrδ^{-/-} mice were subcutaneously implanted with KPC⁻ derived tumor cells engineered to express OVA. On day 10, tumors were directly inoculated with PBS, FACS-sorted PDA-infiltrating γδT cells treated ex-vivo with Rat IgG2b, or PDA-infiltrating γδT cells treated with αPD-L1. On day 15 (l) tumor volume (scale bar=1 cm), (m) the fraction of CD8⁺ OVA Pentamer+ T cells among all CD8⁺ T cells, and (n) OVA Pentamer+ T cell expression of CD107a were recorded (n=5/group; *p<0.05, **p<0.01, ***p<0.001).

FIG. 14. γδT cells do not alter myeloid cell infiltration or function in PDA and localize with αβT cells in the TME. (a) WT and Tcrδ^{-/-} mice were orthotopically implanted with KPC-derived pancreatic tumor cells. Tumors were harvested at 3 weeks and analyzed by flow cytometry. CD11b+ myeloid cells were gated and tested for co-expression of Ly6C and Ly6G. Representative contour plots and quantitative data from 5 mice are shown. For each set of bars for Ly6C⁺, Ly6C⁺Ly6G⁺, Ly6C⁻ Ly6G⁻, and Ly6G⁺, the bars from left to right are WT, and Tcrδ^{-/-} (b, c) CFSE-labeled splenic CD3⁺ T cells were either unstimulated, or stimulated with αCD3/αCD28 alone or in co-culture with orthotopic PDA-infiltrating (b) MDSC or (c) TAMs (5:1 ratio) derived from WT or Tcrδ^{-/-} mice. αPD-L1 was added to select co-culture wells. T cell proliferation was determined by dilution of CFSE on flow cytometry. In (b), the bars from left to right are: Unstim., Stim., +WT MDSC, +WT MDSC+αPD-L1, and Tcrδ^{-/-} MDSC. In (c), the bars from left to right are: Unstim., Stim., +WT Mφ, +WT Mφ+αPD-L1, and Tcrδ^{-/-} Mφ, (d, e) CD3⁺ T cells were either unstimulated, or stimulated with αCD3/αCD28 alone or in co-culture with orthotopic PDA-infiltrating γδT cells (d) MDSC or (e) TAMs (5:1 ratio) +/−αPD-L1 (10 μg/ml). T cells activation was determined by expression of TNF-α. In (d) the bars from left to right are: Unstim., Stim., +MDSC, and +MDSC+αPD-L1. In (e), the bars from left to right are: Unstim., Stim., +Mφ, and Mφ+αPD-L1, (f) Human PDA and (g) orthotopic KPC tumors were co-stained for CD11b/TCRαβ or TCRγ6/TCRαβ. The closest distance between each αβT cell and CD11b⁺ myeloid cell or γδT cell, respectively, were calculated. Representative high and low power images and quantitative data are shown. 10 low power fields were examined per pancreas. (h) Orthotopic KPC tumors were co-stained for DAPI, TCRγ6, PD-L1, and TCRαβ or (i) DAPI, CK19, PD-L1, and TCRαβ and imaged by confocal microscopy. Two representative images of each combination is shown.

FIG. 15. Gemcitabine Enhanced γδ T Cells and Reduced CD3+ Cells in PDA in a Mouse Model. A: a graph showing the percentage of CD3⁺ cells mice treated with gemcitabine and saline control. B: a graph showing the percentage of γδ T cells in mice treating with gemcitabine and saline control. C: a graph showing the percentage of Vγ1⁺ cells in mice treated with gemcitabine and saline control. D: a graph showing the percentage of Vγ4⁺ cells in mice treated with gemcitabine and saline control.

FIG. 16. Reduced Tumor Sizes in γδ T Cell-Knock Out PDA Mice. A: a chart showing tumor volumes in γδ T Cell-knock out (GDT) mice and control mice, both transplanted with MCA38 tumor cells. B: a chart showing tumor weight in γδ T Cell-knock out (GDT) mice and control mice, both transplanted with MCA38 tumor cells.

### DESCRIPTION OF THE DISCLOSURE

Immune suppressive inflammation is paramount for PDA progression. Murine modeling of PDA using animals that endogenously express pancreas-specific oncogenic Kras revealed that pancreatic dysplasia is preceded by and accompanied by vigorous pancreatitis (Hingorani et al., 2003). Moreover, a driving oncogenic mutation alone is insufficient for disease progression and concomitant pancreatitis is necessary for PDA development (Guerra et al., 2007, Cancer cell 11, 291-302). The peri-pancreatic immune infiltrate is rife with immune-suppressive elements that support oncogenesis. In particular, innate immune cells within the tumor microenvironment (TME) are apt at educating adaptive immune effectors towards a tumor-permissive phenotype. APC populations, including M2-polarized TAMs and myeloid dendritic cells, induce the generation of PDA-promoting Th2 cells over Th1 cells that facilitate cytotoxic T lymphocytes (CTL) (Ochi et al., 2012b, J Exp Med 209, 1671-1687; Zhu et al., 2014, Cancer Res 74, 5057-5069).

The present disclosure is based, at least in part, on the unexpected discovery of a specific γδ T cell population which constitutes approximately 40% of tumor-infiltrating T cells in human pancreatic ductal adenocarcinoma (PDA). It was found that recruitment and activation of γδ T cells is contingent on diverse chemokine signals; deletion, depletion, or blockade of γδ T cell recruitment was protective against PDA and resulted in increased infiltration, activation, and Th1-polarization of αβ T cells. While αβ T cells were dispensable to outcome in PDA, they are indispensable mediators of tumor-protection upon γδ T cell ablation. PDA-infiltrating γδ T cells expressed high levels of exhaustion ligands and thereby negated adaptive anti-tumor immunity. Blocking PD-L1 in γδ T cells enhanced CD4⁺ and CD8+ T cell infiltration and immunogenicity and induced tumor protection, suggesting that γδ T cells are critical sources of immune-suppressive checkpoint ligands in PDA. Thus, γδ T cells can be described as central regulators of effector T cell activation in cancer via novel cross-talk.

Accordingly, described herein are methods for treating solid tumors such as PDA via targeting γδ T cells and diagnostic methods for PDA using γδ T cells as biomarkers.

Treating Solid Tumors with γδ T Cell Suppressors

The present disclosure provides methods of treating a solid tumor (e.g., PDA or CRC) in a subject by targeting γδ T cell, e.g., inhibiting γδ T cell activity, depleting γδ T cells, blocking recruitment of γδ T cells to tumor sites, and/or suppressing γδ T cell expansion.

### (i) γδ T Cell Suppressors

γδ T cells are distinctive T cells that contain specific T cell receptors (TCR) on their surface; the TCRs each comprise one gamma (γ) and one delta (δ) chain. γδ1 T cells are γδ T cells that bear the Deltal subunit (δ1) or Delta2 subunit (δ2) of the T cell receptor. γδ T cells play roles in both the innate and adaptive immune responses. Activated γδ T cells release interferon (IFN)-γ and tumor necrosis factor (TNF)-α and exhibit potent anti-tumor activity (Gogoi et al., Indian J Med Res. 2013, 138(5): 755-761). As used herein, tumor-associated γδ T cells refer to the γδ T cell population that is permissive to tumor growth, via, e.g., their suppressive activity on αβ T cells, which are protective against tumor development. Tumor-associated γδ T cells may be infiltrated into a tumor site and/or may be circulating T cells.

The term “γδ T cell suppressor” as used herein refers to a compound that is capable of reducing or eliminating the activity of γδ T cells (e.g., tumor-associated suppressive γδ T cells) either directly or indirectly. The target γδ T cells can be circulating γδ T cells and/or γδ T cells infiltrated into the tumor microenvironment (TME). An agent that reduces the activity of γδ T cells (e.g., tumor-associated γδ T cells) refers to an agent capable of reducing the activity of γδ T cells, for example, reducing the immunosuppressive activity of the γδ T cells, by, e.g., at least 10% (e.g., 20%, 30%, 40%, 50%, 60%, 70%, 80%, 90%, or above). In some instances, such an agent eliminates the activity of γδ T cells, (e.g., no significant activity of γδ T cells is detected in a conventional assay in the presence of the agent). The activity of a candidate γδ T cell suppressor can be determined via conventional assays or assays described herein.

A γδ T cell suppressor for use in the method described herein may be (i) an agent that reduces the level of γδ T cells, particularly tumor-associated suppressive γδ T cells. Such a suppressor may be an antibody specific to γδ T cells (for example, an antibody specific to γδ1 T cells) that depletes γδ T cells. Alternatively, such a suppressor may be an immune cell (e.g., a T cell or an NK cell) expressing a chimeric receptor that comprises an antigen-binding domain specific to the γδ T cells (e.g., specific to a cell surface receptor thereof such as TCR).

A γδ T cell suppressor for use in the method described herein may also be an agent that suppresses γδ T cell activity, for example, the immunosuppressive activity on αβ T cells. Such a suppressor may be an agent (e.g., an antibody or a small molecule) that targets a cell surface receptor of a γδ T cell and blocks the signaling pathway mediated by the γδ T cell receptor and its cognate ligand (e.g., a ligand on another immune cells). γδ T cell surface receptors to be targeted by the suppressor may include TCR (or a subunit thereof) or a checkpoint molecule such as PD-L1. Such a suppressor may also be an agent (e.g., an antibody or a small molecule) that targets the ligand to which the γδ T cell surface receptor binds (e.g., Galectin-9).

Alternatively, a γδ T cell suppressor can be an agent that reduces the expression level of a γδ T cell-associated molecule that mediates the immunosuppressive function of the T cells (both extracellularly and intracellularly), for example, agents that reduce the expression level of one or more immune checkpoint molecule(s), such as PD-L1) or Galectin-9 on γδ T cells (e.g., interfering RNAs).

In other embodiments, a γδ T cell suppressor may be an agent that blocks recruitment of γδ T cells to a tumor site, for example, antibodies specific to chemokines or ligands thereof, such as CCR2, CCL2, or CCR6.

A. Antibodies Suppressing γδ T Cells

In some embodiments, the γδ T cell suppressors described herein are antibodies that suppress γδ T cells, for example, reducing or eliminating γδ T cells and/or inhibiting γδ T cell activities, directly or indirectly. Such antibodies may bind γδ T cells (e.g., γδ1 T cells), thereby reducing/eliminating γδ T cells via, e.g., antibody-mediated cell toxicity (ADCC), and/or blocking the interaction between γδ T cells and other immune cells (for example, αβ T cells). In some examples, the antibody binds (e.g., specifically binds) a TCR (e.g., a TCR containing the delta 1 subunit or a TCR containing the delta 2 subunit) or a component thereof (e.g., a delta 1 subunit or a delta 2 subunit). In other examples, the antibody binds (e.g., specifically binds) a γδ T cell surface molecule that mediates the immunosuppressive activity of the γδ T cell, for example, a checkpoint molecule (e.g., PD-L1) or Galectin-9.

In some instances, antibodies binding to γδ T cells may be a bi-specific or tri-specific T cell engager or NK cell engager, which can form a link between αβ T cells and the target γδ T cells or between NK cells and the target γδ T cells. Such linkage causes the αβ T cells or the NK cells to exert cytotoxic activity on the target γδ T cells, thereby eliminating or reducing the levels of the target γδ T cells. A bi-specific T cell or NK cell engager may be a bi-specific antibody that binds both the target γδ T cell and an αβ T cell or NK cell, for example, a surface receptor of the αβ T cell (e.g., CD3) or NK cell (e.g., CD16). A tri-bispecific T cell or NK cell engager may be a tri-specific antibody that binds the γ chain of the γδ T cells, the δ chain of the γδ T cells, and an the αβ T cell or NK cell, for example, a surface receptor of the αβ T cell (e.g., CD3) or NK cell (e.g., CD16).

The antibody may also be specific to a chemokine or a ligand thereof that plays a role in recruitment of γδ T cells to a tumor site. Examples of chemokines/ligands thereof include, but are not limited to, CCR2, CCL2, and CCR6. Alternatively or in addition, the antibody may block antigenic expansion of γδ T cells.

Galectin-9 is a member of the Galectins family, which has high binding affinity to β-galactoside sugars. Galectin-9 has three different isoforms which differ in the length of the linker region. Exemplary human Galectin-9 polypeptides include those described under GenBank accession no. O00182.2, GenBank accession no. BAB83624.1, and GenBank accession no. BAB83623.1. In some examples, the anti-Galectin-9 antibodies described herein binds the CRD1 domain or the CRD2 domain of Galectin-9.

An antibody (interchangeably used in plural form) as used herein is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

In some embodiments, an antibody as described herein can bind and inhibit a target antigen (e.g., γδ T cells, for example, a cell surface receptor thereof) by at least 50% (e.g., 60%, 70%, 80%, 90%, 95% or greater). The apparent inhibition constant (Ki^{aPP} or K_{i,app}), which provides a measure of inhibitor potency, is related to the concentration of inhibitor required to reduce enzyme activity and is not dependent on enzyme concentrations. The inhibitory activity of the antibody described herein can be determined by routine methods known in the art.

The K_{i,}^{aPP} value of an antibody may be determined by measuring the inhibitory effect of different concentrations of the antibody on the extent of the reaction (e.g., enzyme activity); fitting the change in pseudo-first order rate constant (v) as a function of inhibitor concentration to the modified Morrison equation (Equation 1) yields an estimate of the apparent Ki value. For a competitive inhibitor, the Ki^{app} can be obtained from the y-intercept extracted from a linear regression analysis of a plot of K_{i,}^{aPP} versus substrate concentration.

$$v = A \cdot \frac{\begin{matrix}( [ E \} - [ I \} - {K}_{i}^{app} ) + \\ \sqrt{{( [ E ] - [ I ] - {K}_{i}^{app} )}^{2} + 4 [ E ]} \cdot {K}_{i}^{app}\end{matrix}}{2} \qquad ( Equation 1 )$$

Where A is equivalent to vₒ/E, the initial velocity (vₒ) of the enzymatic reaction in the absence of inhibitor (I) divided by the total enzyme concentration (E).

In some embodiments, the antibody described herein may have a Ki^{app} value of 1000, 900, 800, 700, 600, 500, 400, 300, 200, 100, 50, 40, 30, 20, 19, 18, 17, 16, 15, 14, 13, 12, 11, 10, 9, 8, 7, 6, 5 pM or less for the target antigen or antigen epitope as described herein. In some embodiments, the antibody may have a lower Ki^{app} for a first target (e.g., a human delta 1 subunit of a γδ T cell receptor, a human delta 2 subunit of a γδ T cell receptor, a human Galectin-9, or a human PD-1) relative to a second target (e.g., a mouse γδ T cell receptor, a mouse Galectin-9, or a house PD-1). Differences in Ki^{aPP} (e.g., for specificity or other comparisons) can be at least 1.5, 2, 3, 4, 5, 10, 15, 20, 37.5, 50, 70, 80, 91, 100, 500, 1000, 10,000 or 10⁵ fold. In some examples, the antibody inhibits a first antigen (e.g., a first protein in a first conformation or mimic thereof) better relative to a second antigen (e.g., the same first protein in a second conformation or mimic thereof; or a second protein). In some embodiments, any of the antibodies may be further affinity matured to reduce the Ki^{app} of the antibody to the target antigen or antigenic epitope thereof.

The antibodies described herein can be murine, rat, human, or any other origin (including chimeric or humanized antibodies). Such antibodies are non-naturally occurring, i.e., would not be produced in an animal without human act (e.g., immunizing such an animal with a desired antigen or fragment thereof).

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In one example, the antibody used in the methods described herein is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, and/or six) which are altered with respect to the original antibody, which are also termed one or more CDRs “derived from” one or more CDRs from the original antibody. Humanized antibodies may also involve affinity maturation.

In another example, the antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, the antibodies described herein specifically bind to the corresponding target antigen or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen (e.g., γδ T cell or a cell surface receptor thereof such as TCR, Galectin-9, or PD-L1) is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen. In some embodiments, the antibodies described herein specifically bind to γδ T cells, for example, γδ1 T cells. In some embodiments, the antibodies described herein specifically bind to a Galectin-9 polypeptide, for example, human Galectin-9 or an epitope therein (e.g., the CRD1 or CRD2 regions therein).

In some embodiments, an antibody as described herein has a suitable binding affinity for the target antigen (e.g., γδ T cells). As used herein, “binding affinity” refers to the apparent association constant or K_{A}. The K_{A} is the reciprocal of the dissociation constant (K_{D}). The antibody described herein may have a binding affinity (K_{D}) of at least 10⁻⁵, 10⁻⁶, 10⁻⁷, 10⁻⁸, 10⁻⁹, 10⁻¹⁰ M, or lower for the target antigen or antigenic epitope. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold. In some embodiments, any of the antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

Antibodies capable of binding to γδ T cells, Galectin-9, or a checkpoint molecule as described herein can be made by any method known in the art. See, for example, Harlow and Lane, (1998) Antibodies: A Laboratory Manual, Cold Spring Harbor Laboratory, New York.

In some embodiments, antibodies specific to a target antigen as described herein can be made by the conventional hybridoma technology. The full-length target antigen or a fragment thereof, optionally coupled to a carrier protein such as KLH, can be used to immunize a host animal for generating antibodies binding to that antigen. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, D. W., et al., In Vitro, 18:377-381 (1982). Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the monoclonal antibodies specific to the target antigens described herein. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies capable of inhibiting γδ T cell activity, directly or indirectly. Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a target antigen or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, succinic anhydride, SOCl, or R1N═C═NR, where R and R1 are different alkyl groups, can yield a population of antibodies (e.g., monoclonal antibodies).

If desired, an antibody (monoclonal or polyclonal) of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy in inhibiting the activity of the target antigen. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse® from Amgen, Inc. (Fremont, Calif.) and HuMAb-Mouse® and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Alternatively, antibodies capable of binding to the target antigens as described herein may be isolated from a suitable antibody library via routine practice, for example, using the phage display, yeast display, ribosomal display, or mammalian display technology known in the art.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of VH and VL of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human VH and VL chains having amino acid sequences that are homologous to those of the parent non-human antibody are identified from any antibody gene database using the parent VH and VL sequences as search queries. Human VH and VL acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage or yeast scFv library and scFv clones specific to a target antigen can be identified from the library following routine procedures. Positive clones can be subjected to further screening to identify those that inhibit the activity of the target antigen.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of the target polypeptide have been replaced (swapped) with sequences from a closely related, but antigenically distinct protein (such as another member of the neurotrophin protein family). By assessing binding of the antibody to the mutant target antigen, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art.

In some examples, an antibody as described herein can be prepared by the conventional recombinant technology using a suitable host cell, for example, a mammalian cell line (e.g., CHO cells).

B. Other γδ T Cell Suppressors

In addition to the antibodies described herein, γδ T cell suppressors may be antisense nucleic acid molecules capable of blocking or decreasing the expression of an intra- or extra-cellular molecule of a γδ T cell (e.g., a tumor-associated γδ T cell) that mediates the immunosuppressive activity thereof, for example, a checkpoint molecule (e.g., PD-L1) or Galectin-9. Nucleotide sequences encoding those target molecules are known and are readily available from publicly available databases. See above disclosures. It is routine to prepare antisense oligonucleotide molecules that will specifically bind a target mRNA without cross-reacting with other polynucleotides. Exemplary sites of targeting include, but are not limited to, the initiation codon, the 5′ regulatory regions, the coding sequence and the 3′ untranslated region. In some embodiments, the oligonucleotides are about 10 to 100 nucleotides in length, about 15 to 50 nucleotides in length, about 18 to 25 nucleotides in length, or more. The oligonucleotides can comprise backbone modifications such as, for example, phosphorothioate linkages, and 2′-O sugar modifications well known in the art.

Alternatively, the expression and/or release of any of the target antigens described herein can be decreased using gene knockdown, morpholino oligonucleotides, small interfering RNA (siRNA or RNAi), microRNA or ribozymes, methods that are well-known in the art. RNA interference (RNAi) is a process in which a dsRNA directs homologous sequence-specific degradation of messenger RNA. In mammalian cells, RNAi can be triggered by 21-nucleotide duplexes of small interfering RNA (siRNA) without activating the host interferon response. The dsRNA used in the methods disclosed herein can be a siRNA (containing two separate and complementary RNA chains) or a short hairpin RNA (i.e., a RNA chain forming a tight hairpin structure), both of which can be designed based on the sequence of the target gene. Alternatively, it can be a microRNA.

Optionally, a nucleic acid molecule to be used in the method described herein (e.g., an antisense nucleic acid, a small interfering RNA, or a microRNA) as described above contains non-naturally-occurring nucleobases, sugars, or covalent internucleoside linkages (backbones). Such a modified oligonucleotide confers desirable properties such as enhanced cellular uptake, improved affinity to the target nucleic acid, and increased in vivo stability.

In one example, the nucleic acid has a modified backbone, including those that retain a phosphorus atom (see, e.g., U.S. Pat. Nos. 3,687,808; 4,469,863; 5,321,131; 5,399,676; and 5,625,050) and those that do not have a phosphorus atom (see, e.g., U.S. Pat. Nos. 5,034,506; 5,166,315; and 5,792,608). Examples of phosphorus-containing modified backbones include, but are not limited to, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having 3′-5′ linkages, or 2′-5′ linkages. Such backbones also include those having inverted polarity, i.e., 3′ to 3′, 5′ to 5′ or 2′ to 2′ linkage. Modified backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In another example, the nucleic acid used in the disclosed methods includes one or more substituted sugar moieties. Such substituted sugar moieties can include one of the following groups at their 2′ position: OH; F; O-alkyl, S-alkyl, N-alkyl, O-alkenyl, S-alkenyl, N-alkenyl; O-alkynyl, S-alkynyl, N-alkynyl, and O-alkyl-O-alkyl. In these groups, the alkyl, alkenyl and alkynyl can be substituted or unsubstituted C₁ to C₁₀ alkyl or C₂ to C₁₀ alkenyl and alkynyl. They may also include at their 2′ position heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide. Preferred substituted sugar moieties include those having 2′-methoxyethoxy, 2′-dimethylaminooxyethoxy, and 2′-dimethylaminoethoxyethoxy. See Martin et al., Helv. Chim. Acta, 1995, 78, 486-504.

In yet another example, the nucleic acid includes one or more modified native nucleobases (i.e., adenine, guanine, thymine, cytosine and uracil). Modified nucleobases include those described in U.S. Pat. No. 3,687,808, The Concise Encyclopedia Of Polymer Science And Engineering, pages 858-859, Kroschwitz, J. I., ed. John Wiley & Sons, 1990, Englisch et al., Angewandte Chemie, International Edition, 1991, 30, 613, and Sanghvi, Y. S., Chapter 15, Antisense Research and Applications, pages 289-302, CRC Press, 1993. Certain of these nucleobases are particularly useful for increasing the binding affinity of the antisense oligonucleotide to its target nucleic acid. These include 5-substituted pyrimidines, 6-azapyrimidines and N-2, N-6 and O-6 substituted purines (e.g., 2-aminopropyl-adenine, 5-propynyluracil and 5-propynylcytosine). See Sanghvi, et al., eds., Antisense Research and Applications, CRC Press, Boca Raton, 1993, pp. 276-278).

Any of the nucleic acids can be synthesized by methods known in the art. See, e.g., Caruthers et al., 1992, Methods in Enzymology 211, 3-19, Wincott et al., 1995, Nucleic Acids Res. 23, 2677-2684, Wincott et al., 1997, Methods Mol. Bio. 74, 59, Brennan et al., 1998, Biotechnol Bioeng., 61, 33-45, and Brennan, U.S. Pat. No. 6,001,311. It can also be transcribed from an expression vector and isolated using standard techniques.

In other embodiments, the γδ T cell suppressor described herein can be a non-antibody compound that directly or indirectly reduces, inhibits, neutralizes, or abolishes the biological activity of γδ T cells. Such an inhibitory compound should exhibit any one or more of the following characteristics: (a) reduces the level of tumor-associated γδ T cells; (b) reduces the expression level of molecules such as immune check point molecules that are involved in the immunosuppressive activity of γδ T cells; and/or (c) blocks suppression of αβ T cells by γδ T cells (for example, by activating checkpoint signaling). Such suppressors may reduce the level of γδ T cells/eliminate the γδ T cells directly, or block the immunosuppressive activity of the γδ T cells activity. For example, the suppressor may block the Galectin-9 signaling pathway. In some instances, the non-antibody compounds may be mutants of γδ T cell surface receptors or mutants of their cognate ligands, which are capable of binding to the cell surface receptor/ligand and blocking their bioactivity.

In some instances, the γδ T cell suppressor may be an immune cell such as a T cell (a CD4⁺ or CD8⁺ T cell) or an NK cell that expresses a chimeric antigen receptor (CAR) targeting γδ T cells. Such a CAR construct may comprise an extracellular domain specifically binds a γδ T cell and eliminate the target γδ T cell via CAR-T cell-mediated cell toxicity.

In other embodiments, the γδ T cell suppressor may be small molecule compounds that suppress the activity of γδ T cells. Such a small molecule compound may have a molecular weight of about any of 100 to 20,000 daltons, 500 to 15,000 daltons, or 1000 to 10,000 daltons. Such small molecule compounds may be obtained from compound libraries. The libraries can be spatially addressable parallel solid phase or solution phase libraries. See, e.g., Zuckermann et al. J. Med. Chem. 37, 2678-2685, 1994; and Lam Anticancer Drug Des. 12:145, 1997. Methods for the synthesis of compound libraries are well known in the art, e.g., DeWitt et al. PNAS USA 90:6909, 1993; Erb et al. PNAS USA 91:11422, 1994; Zuckermann et al. J. Med. Chem. 37:2678, 1994; Cho et al. Science 261:1303, 1993; Carrell et al. Angew Chem. Int. Ed. Engl. 33:2059, 1994; Carell et al. Angew Chem. Int. Ed. Engl. 33:2061, 1994; and Gallop et al. J. Med. Chem. 37:1233, 1994. Libraries of compounds may be presented in solution (e.g., Houghten Biotechniques 13:412-421, 1992), or on beads (Lam Nature 354:82-84, 1991), chips (Fodor Nature 364:555-556, 1993), bacteria (U.S. Pat. No. 5,223,409), spores (U.S. Pat. No. 5,223,409), plasmids (Cull et al. PNAS USA 89:1865-1869, 1992), or phages (Scott and Smith Science 249:386-390, 1990; Devlin Science 249:404-406, 1990; Cwirla et al. PNAS USA 87:6378-6382, 1990; Felici J. Mol. Biol. 222:301-310, 1991; and U.S. Pat. No. 5,223,409).

### (ii) Pharmaceutical Compositions

Any of the γδ T cell suppressors (e.g., antibodies, antisense nucleic acids, polypeptide mutants, or small molecule compounds) as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The γδ T cell suppressors may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(v nylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT′ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

(iii) Therapeutic Applications

To practice the method disclosed herein, an effective amount of the γδ T cell suppressor described herein, formulated in a suitable pharmaceutical composition as also described herein, can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a solid tumor, such as pancreatic duct adenocarincoma (PDA), colorectal cancer (CRC), melanoma, breast cancer, lung cancer (e.g., non-small cell lung cancer, NSCLC, and small cell lung cancer, SCLC), upper and lower gastrointestinal malignancies (e.g., esophageal, gastric, and hepatobiliary), squamous cell head and neck, genitourinary, and sarcomas. A subject having a solid tumor can be identified by routine medical examination, e.g., laboratory tests, diagnostic biopsy, organ functional tests, surgical intervention, a suitable imaging modality, or a combination thereof. Such a subject may also be identified by the diagnostic method described herein. A subject suspected of having any of such target disease/disorder might show one or more symptoms of the disease/disorder. A subject at risk for the disease/disorder can be a subject having one or more of the risk factors for that disease/disorder. In some embodiments, the subject to be treated by the method described herein may be a human cancer patient who has undergone or is subjecting to an anti-cancer therapy, for example, chemotherapy, radiotherapy, immunotherapy, surgery, or administration of a targeted agent, which is directed to a specific molecule involved in the target cancer. Examples of targeted agents include small molecule tyrosine kinase inhibitors, including, but not limited to, Imatinib (Gleevec®/Glivec®), Gefitinib (Iressa®), Erlotinib (OSI-774, Tarceva®), Lapatinib (GW-572016, Tykerb®), Canertinib (CI-1033), Sunitinib (SU 11248, Sutent®), Zactima (ZD6474), Vatalanib (PTK787/ZK 222584), Sorafenib (Bay 43-9006, Nexavar®), Leflunomide (SU101, Arava®), Dasatinib (Sprycel®), Regorafenib (Bay 73-4506, Stivarga®), Nilotinib (Tasigna®), Pazopanib (Votrient®), Palbociclib (Ibrance®), and Ribociclib (Kisqali®).

As used herein, “an effective amount” refers to the amount of each active agent required to confer therapeutic effect on the subject, either alone or in combination with one or more other active agents. In some embodiments, the therapeutic effect is reduced γδ T cell presence, including reduced γδ T cell activity or expression, or enhanced anti-tumor immunity via, e.g., enhanced αβ T cell activity and/or reduced activity of γδ T cells, e.g., circulating γδ T cells or γδ T cells infiltrated into the TME. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for a γδ T cell suppressor such as an antibody as described herein may be determined empirically in individuals who have been given one or more administration(s) of the suppressor. Individuals are given incremental dosages of the suppressor. To assess efficacy of the suppressor, an indicator of the disease/disorder can be followed.

Generally, for administration of any of the antibodies described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. In some embodiments, the dose is a flat dose, which may range from about 50 mg to 100 mg to 150 mg to 200 mg to 250 mg to 300 mg or more, depending on the factors mentioned above. In some instances, the flat dose may be 100 mg or 200 mg. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg 10 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 10 mg/kg (such as about 3 μg/mg, about 10 μg/mg, about 30 μg/mg, about 100 μg/mg, about 300 μg/mg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, and about 10 mg/kg) may be used. In other embodiments, flat dosing ranging from about 50 mg to about 300 mg (such as about 50 mg, about 100 mg, about 125 mg, about 150 mg, about 175 mg, about 200 mg, about 225 mg, about 250 mg, about 275 mg, and about 300 mg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

For the purpose of the present disclosure, the appropriate dosage of a γδ T cell suppressor as described herein will depend on the specific suppressor employed, the type and severity of the solid tumor, whether the suppressor is administered for preventive or therapeutic purposes, previous therapy, the patient's clinical history and response to the antagonist, and the discretion of the attending physician. Typically the clinician will administer an antibody, until a dosage is reached that achieves the desired result. In some embodiments, the desired result is a decrease in thrombosis. Methods of determining whether a dosage resulted in the desired result would be evident to one of skill in the art.

Administration of one or more antagonists can be continuous or intermittent, depending, for example, upon the recipient's physiological condition, whether the purpose of the administration is therapeutic or prophylactic, and other factors known to skilled practitioners. The administration of the antagonist may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

In some embodiments, the antibodies described herein are administered to a subject in need of the treatment at an amount sufficient to inhibit the γδ T cell activity by at least 20% (e.g., 30%, 40%, 50%, 60%, 70%, 80%, 90% or greater) in vivo. In other embodiments, the antibodies are administered in an amount effective in reducing the activity level of a target antigen (e.g., Galectin-9) by at least 20% (e.g., 30%, 40%, 50%, 60%, 70%, 80%, 90% or greater).

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Therapeutic compositions containing a polynucleotide (e.g., those encoding the antibodies described herein) are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The therapeutic polynucleotides and polypeptides described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

The particular dosage regimen, i.e., dose, timing and repetition, used in the method described herein will depend on the particular subject and that subject's medical history.

In some embodiments, more than one antibody, or a combination of an antibody and another suitable therapeutic agent, may be administered to a subject in need of the treatment. The antibody can also be used in conjunction with other agents that serve to enhance and/or complement the effectiveness of the agents.

Treatment efficacy for a target disease/disorder can be assessed by methods well-known in the art.

### (iv) Combined Therapy

Any of the γδ T cell suppressors described herein may be utilized in conjunction with one or more other types of anti-cancer therapy, such as chemotherapy, surgery, radiation, gene therapy, or a treatment involving one or more targeted agents such as those described herein. Such therapies can be performed simultaneously or sequentially (in any order) with the immunotherapy according to the present disclosure.

When co-administered with an additional therapeutic agent, suitable therapeutically effective dosages for each agent may be lowered due to the additive action or synergy.

In some embodiments, the γδ T cell suppressor can be combined with other immunomodulatory treatments such as, e.g., inhibitors of a checkpoint molecule (e.g., PD-1, PD-L1, PD-L2, CDLA-4, LAG3, TIM-3, or A2aR), agonists of a co-stimulatory receptor (e.g., DX40, GITR, CD137, CD40, CD27, and ICOS), inhibitors of an innate immune cell target (e.g., KIR, NKG2A, CD96, TLR, and IDO). In some embodiments, the γδ T cell suppressor is administered with an anti-PD-L1 antibody. Without being bound by theory, it is reported herein that γδ T cell suppressors can release the inhibition of αβ T cells, providing anti-tumor protection and may enhance immune surveillance against tumor cells by, e.g., activating CD4⁺ and/or CD8⁺ T cells. Thus, combined use of a γδ T cell suppressor and an immunomodulatory agent such as those described herein would be expected to significantly enhance anti-tumor efficacy.

In other embodiments, the γδ T cell suppressor described herein can also be co-used with a chemotherapeutic agent or regimen, including alkylating agents (e.g., cyclophosphamide, ifosfamide, melphalan (L-sarcolysin) and chlorambucil)anthracyclines, cytoskeletal disruptors (Taxanes), epothilones, histone deacetylase inhibitors, inhibitors of topoisomerase I, inhibitors of topoisomerase II, kinase inhibitors, nucleotide analogs and precursor analogs, peptide antibiotics (e.g., dactinomycin (actinomycin D), daunorubicin (daunomycin; rubidomycin), doxorubicin, bleomycin, plicamycin (mithramycin) and mitomycin (mitomycin C)), platinum-based agents, retinoids, vinca alkaloids (e.g., vinblastine (VLB)) and derivatives thereof, or FOLFOXIRI (a thermotherapy regimen including folinic acid, fluorouracil, oxaliplatin, and irinotecan). Other cancer chemotherapeutic agents include ethylenimines and methylmelamines such as hexamethylmelamine, thiotepa; alkyl sulphonates such as busulfan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide). Further non-limiting examples include: (i) anti-angiogenic agents (e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000)); (ii) a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof; and (iii) chemotherapeutic compounds such as, e.g., pyrimidine analogs (5-fluorouracil, floxuridine, capecitabine, gemcitabine and cytarabine (cytosine arabinoside)), purine analogs (e.g., mercaptopurine (6-mercaptopurine; 6-MP), thioguanine (6-thioguanine; TG) and pentostatin (2′-deoxycoformycin)), folate antagonists and related inhibitors (methotrexate (amethopterin), mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones, and navelbine, epidipodophyllotoxins (etoposide and teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin (daunomycin; rubidomycin), doxorubicin (adriamycin), idarubicin, anthracyclines, mitoxantrone, bleomycin, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, mitoxantrone, topotecan, and irinotecan), corticosteroids (cortisone, dexamethasone, hydrocortisone, methylprednisolone, prednisone, and prednisolone); growth factor signal transduction kinase inhibitors; mitochondrial dysfunction inducers and caspase activators; and chromatin disruptors. Also included are biological response modifiers such as interferon alphenomes. Further miscellaneous agents include platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); taxol and analogues/derivatives.

Additional useful agents can be found in, e.g., Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

It was reported that chemotherapy (e.g., gemcitabine) and/or immune therapy could enhance the level of immune modulators such as checkpoint molecules, resulting in suppressed immunity against tumor cells. Erisson et al., J. Translational Medicine (2016), 14:282; Grabosch et al., J. ImmunoTherapy of Cancer (2015), 3(suppl 2): P302; and Azad et al., EMBO J. (2016). Here, γδ T cell suppressors were found to reprogram immune responses targeting tumor cells, particularly in PDA. As such, the co-use of a γδ T cell suppressor and a chemotherapeutic agent (e.g., gemcitabine) or immunotherapeutic agent (e.g., anti-PD-L1 antibody) would be expected to result in synergistic therapeutic activity against solid tumors such as PDA or CRC.

### Kits for Use in Treating Solid Tumor

The present disclosure also provides kits for use in treating or alleviating a solid tumor such as PDA and CRC. Such kits can include one or more containers comprising a γδ T cell suppressor, e.g., any of those described herein, and optionally a second therapeutic agent to be co-used with the γδ T cell suppressor, which is also described herein.

In some embodiments, the kit can comprise instructions for use in accordance with any of the methods described herein. The included instructions can comprise a description of administration of the γδ T cell suppressor, and optionally the second therapeutic agent, to treat, delay the onset, or alleviate a target disease as those described herein. The kit may further comprise a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of a γδ T cell suppressor generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating a solid tumor such as PDA or CRC. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a γδ T cell suppressor as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### Methods for Diagnosing Solid Tumors

Also described herein are methods for determining the presence and/or measuring the level of γδ T cells in a biological sample obtained from a subject who is suspected of having a solid tumor such as PDA or CRC. The γδ T cell level thus determined may be used as a biomarker for assessing whether the subject has or is at risk for PDA or CRC, or for assessing treatment efficacy of an anti-PDA or anti-CRC treatment on that subject. In some embodiments, the γδ T cells are effector memory γδ T (TEM) cells. In some embodiments, the γδ T cells are Vγ9⁻ cells.

Such an assay method can comprise at least the following steps: (i) obtaining a biological sample from a subject (e.g., a human patient) suspected of having solid tumor such as PDA or CRC; and (ii) measuring the level and/or of immunosuppressive γδ T cells in the biological sample. Optionally, the level of a checkpoint molecule (for example, PD-L1), the level of Galectin-9, or both in the biological sample can also be measured.

The method may further comprise identifying the subject as having or at risk for the solid tumor if the γδ T cell level thus measured is higher than the γδ T cell level of a control subject (e.g., a PDA-free or CRC-free subject of the same species). A therapy for solid tumor such as PDA, e.g., those described herein or known in the art, can then be applied to the subject, if the subject is identified as having or at risk for PDA or CRC.

A subject suspected of having a solid tumor such as PDA or CRC may exhibit one or more symptoms associated with the solid tumor, for example, jaundice and related laboratory and clinical symptoms, dark urine, light-colored or greasy stools, itchy skin, belly or back pain, weight loss and poor appetite, nausea and vomiting, gallbladder or liver enlargement, and/or blood clots. Such a subject (e.g., a human patient) may be identified by routine medical procedures.

A suitable biological sample can be obtained from a subject as described herein via routine practice. Non-limiting examples of biological samples include fluid samples such as blood (e.g., whole blood, plasma, or serum), urine, and saliva, and solid samples such as tissue (e.g., skin, lung, nasal) and feces. Such samples may be collecting using any method known in the art or described herein, e.g., buccal swab, nasal swab, venipuncture, biopsy, urine collection, or stool collection. In some embodiments, the biological sample is a peripheral blood sample. In some embodiments, the biological sample is a blood sample comprising one or more populations of immune cells. In other embodiments, the biological sample may be a tissue biopsy sample, which may be obtained from a suspected tumor site from the subject.

For prognosis purposes, any of the exemplary samples as described herein (e.g., blood samples or tissue samples) can be obtained from a subject prior to a treatment of a solid tumor (e.g., PDA or CRC), after the treatment, and/or during the course of the treatment.

In some embodiments, the sample may be processed or stored. Exemplary processing includes, for example, cell lysis and extraction of materials from the lysate (e.g., DNA, RNA, or protein). Exemplary storage includes, e.g., adding preservatives to the sample and/or freezing the sample.

The level and/or unique characteristics of immunosuppressive γδ T cells, optionally also the level of a checkpoint molecule (e.g., PD-L1) and/or Galectin-9, in a biological sample can be measured using an antibody that specifically binds to the γδ T cells, or optionally an antibody specific to the checkpoint molecule and an antibody specific to Galectin-9. The level of the checkpoint molecule, and/or Galectin-9 may also be determined as the level of proteins in the sample, the level of mRNAs in the sample, or the activity level of the molecule in the sample, or a combination thereof. Assays for measuring levels of mRNA, protein and activity are known in the art and described herein, e.g., including probe-based assays, array-based assays, PCR-based assays, bead-based assays, immuno-based assays, sequencing, bisulfate assays, etc. (see, e.g., Molecular Cloning: A Laboratory Manual, J. Sambrook, et al., eds., Fourth Edition, Cold Spring Harbor Laboratory Press, Cold Spring Harbor, N.Y., 2012; Current Protocols in Molecular Biology, John Wiley & Sons, Inc., New York; Current Protocols in Gene Expression, John Wiley & Sons, Inc., New York; Microarray Methods and Protocols, R. Matson, CRC Press, 2012; Antibodies: A Laboratory Manual, Cold Spring Harbor Laboratory Press, 2^{nd} ed., 2013).

In some examples, the level of the specific protein in a biological sample, such as a blood sample of a tissue sample, is measured via a suitable method. Exemplary protein level assays include, but are not limited to, immunoassays (e.g., Western blot or enzyme-linked immunosorbent assay (ELISA)) and multiplex bead-based assays. Such assays are known in the art and commercially available. In some examples, the cell-surface expression level of γδ TCRs is measured using a suitable method known in the art or described herein. Such assays may involve the use of a suitable antibody specific to γδ TCR, e.g., those described herein.

In other examples, a level of mRNA is determined in a conventional method or a method described herein. Exemplary mRNA level assays include, but are not limited to probe-based assays (e.g., northern blots, nuclease protection assays, in situ hybridization), array-based assays (e.g., microarrays), PCR-based assays (e.g., quantitative PCR), multiplex bead-based assays (e.g., commercially-available Luminex® technology such as xMAP® and xTAG®, Illumina), and sequencing-based assays. Such assays are known in the art and commercially available.

In some examples, the activity level of a selected compound in a biological sample is measured via a suitable method. Exemplary activity level assays include assays for measuring levels of factors secreted by γδ T cells, for example, CCL2, EGF, granzyme A, granzyme B, or IFN-γ.

In a further example, the level of γδ T cells of a sample (and optionally the level of the checkpoint molecule and/or Galectin-9) can be assessed using the Histo (H)-score approach, which is a method known in the art to assess the extent of nuclear immunoreactivity. Briefly, the staining intensity (0, 1+, 2+, or 3+) is determined for each cell in a fixed field. The percentage of cells at each staining intensity level is calculated, and an H-score is assigned using the following formula:

[1×(% cells 1+)+2×(% cells 2+)+3×(% cells 3+)]

Therefore, the H-score can range from 0-300. A program, such as X-tile, can then be used to establish cutoffs within the calculated range of the data. For example, H score cutoffs that correlate with survival can be determined, which can then be validated in a validation data set.

In another example, the level of γδ T cells in a fixed field can be assessed using the intensity score method, which is also well developed in the art.

The γδ T cell level of a biological sample obtained from a subject as described herein can be relied on to determine whether the subject has or at risk for PDA. If the subject is a PDA patient under a treatment of PDA, the change of γδ T cell levels before and after the treatment, or during the course of the treatment, could be relied on to evaluate the treatment efficacy on that subject. In some examples, the γδ T cell level of the candidate subject can be compared with a pre-determined value as described herein, or the γδ T cell level of a control subject, which can be a subject of the same species and free of PDA or CRC. Optionally, the control subject has matched age, gender, and other physical features as the candidate subject. An elevated level of γδ T cells in the biological sample as compared with the pre-determined value or the γδ T cell level of the control subject indicates that the subject has or at risk for PDA or CRC. Alternatively, a decrease of γδ T cells in a subject undergoing an anti-PDA therapy or anti-CRC therapy after the treatment of along the course of the treatment is indicative of treatment efficacy.

As used herein, “an elevated level of γδ T cells” means that the level of γδ T cells is above a pre-determined value, such as a pre-determined threshold or the level of γδ T cells in a control subject as described herein, e.g., at least 20%, 30%, 40%, 50%, 60%, 70%, 80%, 90%, 100%, 2-fold, 5-fold, 10-fold, 50-fold, or 100-fold higher than the pre-determined value or the level of the control subject. An elevated level of γδ T cells also includes increasing a phenomenon from a zero state (e.g., no or undetectable γδ T cells in a control) to a non-zero state (e.g., some γδ T cells or detectable γδ T cells in a sample).

A pre-determined value can be the γδ T cell level in a control sample (a controlled level), which can be measured using any of the methods known in the art or described herein. In some examples, the pre-determined value is measured by the same method applied for measuring the γδ T cell level in a biological sample. The control level may be a level of the γδ T cells in a control sample, control subject, or a population of control subjects. The control may be (or may be derived from) a normal subject (or normal subjects).

Normal subjects, as used herein, refer to subjects that are apparently healthy and show no signs or symptoms of PDA or CRC (free of PDA or free of CRC). The population of control subjects may therefore be a population of normal subjects.

It is to be understood that the methods provided herein do not require that a control level be measured every time a subject is tested. Rather, in some embodiments, it is contemplated that control levels are obtained and recorded and that any test level is compared to such a pre-determined level. The pre-determined level may be a single-cutoff value or a range of values.

By comparing the γδ T cell level(s) of one or more biological samples obtained from a subject and the pre-determined value as described herein, the subject can be identified as having or at risk for PDA or CRC. Further, decrease of γδ T cells during a course of treatment is indicative that the treatment is effective on the subject.

A subject identified by any of the diagnostic methods described herein may be treated by a conventional anti-PDA therapy or conventional anti-CRC therapy or any of the treatment methods described herein.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Role of γδ T Cells in Pancreatic Cancer

γδT cells have not been well-characterized in PDA and their role in the programming of the TME remains ill-defined. The instant study revealed that γδT cells (almost exclusively Vγ4⁺Vγ1⁻ cells) are pervasive in human and murine PDA and tumor infiltration with γδT cells promotes oncogenic progression whereas genetic deletion, therapeutic depletion, and blockade of recruitment of γδT cells markedly delays morphologic transformation of the pancreas and increases median animal survival by nearly one year in a slowly progressive model of PDA. In contrast to these findings, γδT cells have long been considered potent anti-tumor entities in diverse tumor subtypes (Cordova et al., 2012, PLoS One 7, e49878; Todaro et al., 2009, J Immunol 182, 7287-7296). In melanoma, renal cell cancer, and colon cancer the putative protective effects of γδT cells have led to strategies employing exogenous activation of γδT cells to maximize their tumoricidal activity in vivo (Gao et al., 2003, The Journal of experimental medicine 198, 433-442; Girardi et al., 2001, Science 294, 605-609; Lanca and Silva-Santos, 2012, Oncoimmunology 1, 717-725). While the instant findings are ostensibly paradoxical to the described function of γδT cells in these cancer models, it has been demonstrate herein that the γδT cells in PDA exhibit a unique phenotype. Most interestingly, PDA-infiltrating γδT cells express substantial FoxP3 which is absent in spleen γδT cells from the same animals.

Surprisingly, it was demonstrated that deletion of γδT cells in PDA does not influence the fraction of myeloid cells in the TME nor does it affect their functional capacity to suppress T cell proliferation. Further, the in vitro correlative studies suggested that secreted factors in γδT cell conditioned media were non-inhibitory to CD4⁺ and CD8⁺ T cell activation.

Overall, it was demonstrated that γδT cells create an immune-suppressive adaptive TME through checkpoint receptor ligation in tumor-infiltrating αβT cells. Deletion of γδT cells in PDA results in a robust influx of CD4⁺ and CD8⁺ T cells. Furthermore, in the absence of γδT cells, CD4⁺ T cells exhibit accentuated Th1-differentiation and CD8⁺ T cells exhibit a heightened cytotoxic phenotype. Moreover, whereas deletion of CD4⁺ and CD8⁺ T cells did not accelerate tumor progression in γδT cell-competent hosts, in Tcrδ^{-/-} mice αβT cell deletion nearly tripled the rate of PDA growth. This observation supports the notion that αβT cells are entirely dispensable in PDA, but are reprogramed into powerful anti-tumor entities in the absence of γδT cells.

The present study also showed that γδT cells express considerably higher levels of PD-L1 and Galectin-9 in PDA than cancer cells. More importantly, it was demonstrated that γδT cells are important contributors to PD-L1 and Galectin-9 induced T cell exhaustion in the TME based on our observation that inhibition of PD-L1 and Galectin-9 in PDA is protective in vivo in the presence of γδT cells, whereas in absence of γδT cells PD-L1 or Galectin-9 blockade offers no additional tumor-protective benefit. Even more, PD-L1 or Galectin-9 blockade expand and potently activate PDA-infiltrating CD4⁺ and CD8⁺ T cells in γδT cell-competent hosts but do not enhance αβT cell immunogenicity in the absence of γδT cells. It is surprising that checkpoint receptor blockade would not have potency in Tcrδ^{-/-} mice considering the substantial myeloid cell infiltrate in PDA (Liou et al., 2015, Cancer discovery 5, 52-63; Pylayeva-Gupta et al., 2012, Cancer Cell 21, 836-847).

In summary, it was shown here that PDA-infiltrating γδT cells are a highly influential lymphocyte subset in human and murine PDA which promote pancreatic oncogenesis and reduce survival via novel cross-talk with the adaptive immune compartment. These data implicate γδT cells as high-yield targets for the development of experimental therapeutics in PDA and has potential implications for the mechanistic progression of oncogenesis in other cancer subtypes. Finally, γδT cells may have prognostic significance in PDA, and can be used for predicting response to immunotherapeutic regimens.

### Experimental Procedures

(I) Animals and In Vivo Procedures

C57BL/6 (H-2Kb), C57BL/6-Trdc^{tm1Mal}, CCR2^{-/-}, CCR5^{-/-}, CCR6^{-/-}, CCL2^{-/-}, and B6.129P2-Tcrd^{tm1Mom}/J (Tcrδ^{-/-}) mice were purchased from Jackson Labs (Bar Harbor, Me.). KC mice, which develop pancreatic neoplasia endogenously by expressing mutant Kras, were generated by crossing LSL-Kras^{G12D} and p48^{Cre} mice (Hingorani et al., 2003, Cancer Cell 4, 437-450). Tcrδ^{-/-} mice were crossed with KC mice to generate KC;Tcrδ^{-/-} animals. For orthotopic tumor challenge, mice were administered intra-pancreatic injections of tumor cells derived from KPC mice (1×10⁵ cells in Matrigel) and sacrificed at 3 weeks as described (Zambirinis et al., 2015, The Journal of experimental medicine 212, 2077-2094). For subcutaneous tumor challenge, KPC-derived tumor cells (1×10⁶) engineered to express OVA using pCI-neo-cOVA (gift of Maria Castro; Addgene plasmid #25097) were administered to the flanks of mice (Yang et al., 2010, Proceedings of the National Academy of Sciences of the United States of America 107, 4716-4721). In select experiments, FACS-sorted PDA-infiltrating γδT cells were orthotopicaly transferred (8×10⁵) together with tumor cells or directly inoculated into subcutaneous tumors (3×10⁵). In other experiments, animals were treated twice weekly with i.p. injection of neutralizing mAbs directed against TCR Vγ4 (UC3-10A6, 8 mg/kg), PD-L1 (10F.9G2, 5 mg/kg), or Galectin-9 (RG9-1, 6 mg/kg; all BioXCell, West Lebanon, N.H.). In select experiments CD4 (GK1.5; BioXCell) or CD8 (53-6.72; Monoclonal Antibody Core Facility, Sloan Kettering Institute, New York, N.Y.) T cells were depleted using previously described regimens (Bedrosian et al., 2011, Gastroenterology 141, 1915-1926 e1911-1914). Acute pancreatitis was induced using a regimen of seven hourly i.p. injections of caerulein (50 μg/kg; Sigma, St. Louis, Mo.) for two consecutive days as we have described (Bedrosian et al., 2011, Gastroenterology 141, 1915-1926 e1911-1914). Serum amylase and lipase levels were measured using commercial kits (Sigma) according to the manufacturer's instructions. Animal procedures were approved by the NYU School of Medicine IACUC.

(II) Human and Murine Cellular Isolation

Pancreatic leukocytes were harvested from mouse PDA as described previously (Ochi et al., 2012a, The Journal of clinical investigation 122, 4118-4129). Briefly, pancreata were resected in total and placed in ice-cold PBS with 1% FBS with Collagenase IV (1 mg/mL; Worthington Biochemical, Lakewood, N.J.) and DNAse I (2 U/mL; Promega, Madison, Wis.). After mincing, tissues were incubated in the same solution at 37° C. for 30 minutes with gentle shaking. Specimens were passed through a 70 μm mesh, and centrifuged at 350 g for 5 minutes. Human pancreatic tissues and PBMC were collected under an IRB approved protocol. Human pancreatic leukocytes were prepared in a similar manner to mice. PBMC were isolated by overlaying whole blood diluted 3:1 in PBS over an equal amount of Ficoll (GE Healthcare, Princeton, N.J.). Cells were then centrifuged at 2100 RPM and the buffy coat harvested as we have described (Rehman et al., 2013, J Immunol 190, 4640-4649).

(III) Flow Cytometry and FACS Sorting

Cells were suspended in ice-cold PBS with 1% FBS. After blocking FcγRIII/II with an anti-CD16/CD32 mAb (eBioscience, San Diego, Calif.), cell labeling was performed by incubating 10⁶ cells with 1 μg of fluorescently conjugated antibodies directed against murine CD45 (30-F11), CD3 (17A2), CD4 (RM4-5), CD8 (53-6.7), Tcrγ/δ (GL3), CD62L (MEL-14), FasL (MFL3), VΓ1 (2.11), Vγ4 (UC3-10A6), NK1.1 (PK136), CD39 (Duha59), CCR2 (K036C2), CCR5 (HM-CCR5), CCR6 (292L17), CD44 (IM7), JAML (4E10), NKG2D (CX5), CD11b (M1/70), Gr1 (RB6-8C5), PD-1 (29F.1A12), ICOS (15F9), Tcrα/β (B1), TLR4 (SA15-21), TNF-α (MP6-XT22), IL-13 (eBio13A), IL-17 (TC11-18H10.1), IL-10 (JESS-16E3), INF-γ (XMG1.2), Granzyme B (12-8898-80), PD-L1 (10F.9G2), Galectin-9 (4G9-35), B7-1 (16-10A1), B7-2 (PO3), ICOSL (HK5.3), OX-40L (RM134L), CD107A (1D4B), OX-40 (OX-86; all Biolegend, San Diego, Calif.), TLR7 (IMG-581A), TLR9 (26C593.2; both Imgenex, San Diego, Calif.), T-bet (eBio4B10), GATA-3 (TWAJ), and FoxP3 (FJK-16s; all eBioscience). OVA-restricted CD8⁺ T cell proliferation was determined using an H-2kb SIINFEKL OVA Pentamer (ProImmune, Oxford, United Kingdom). Intracellular staining was performed using the FoxP3 Fixation/Permeabilization Solution Kit (eBiosciences). Analysis of human cells was performed using fluorescently conjugated antibodies directed against CD45 (HI30), CD3 (SK7), CD45RA (HI100), CD27 (O323), CD62L (DREG-56), CD14 (HCD14), CD15 (W6D3), CD11c (3.9), Vγ9 (B3; all Biolegend), Tcrγ/δ (B1.1; eBioscience). Flow cytometry was performed on the LSR-II (BD Biosciences, Franklin Lakes, N.J.). Cytokine levels in cell culture supernatant were measured using a cytometric bead array (BD Biosciences). FACS-sorting was performed on the SY3200 (Sony, Tokyo, Japan). Data were analyzed using FlowJo (Treestar, Ashland, Oreg.).

(IV) Statistical Analysis

Data is presented as mean+/−standard error. Survival was measured according to the Kaplan-Meier method. Statistical significance was determined by the Student's t test and the Wilcoxon test using GraphPad Prism 6 (GraphPad Software, La Jolla, Calif.). P-values <0.05 were considered significant.

(V) Western Blotting

Cells or tissues were lysed in ice-cold RIPA buffer. Total protein was quantified using the BioRad DC Protein Assay according to the manufacturer's instructions (BioRad, Hercules, Calif.). Western blotting was performed as described previously with minor modifications (Ochi et al., 2012, The Journal of clinical investigation 122, 4118-4129). Briefly, 10% Bis-Tris polyacrylamide gels (NuPage; Invitrogen, Carlsbad, Calif.) were equiloaded with 10-30m protein, electrophoresed at 200 V and electrotransferred to PVDF membranes. After blocking with 5% BSA, membranes were probed with primary antibodies to Bcl-XL (54H6), Rb (C-15), c-Myc (9E10), PTEN (26H9), p53 (DO-7), and β-actin (8H10D10), all Cell Signaling, Beverly, Mass. Blots were developed by ECL (Thermo Scientific, Asheville, N.C.).

(VI) Histology, Immunohistochemistry, and Microscopy

For histological analysis, pancreatic specimens were frozen in OCT medium or fixed with 10% buffered formalin, dehydrated in ethanol, embedded with paraffin, and stained with H&E or Gomori's Trichrome. The fraction of preserved acinar area was calculated as previously described (Ochi et al., 2012, The Journal of clinical investigation 122, 4118-4129). The fraction and number of ducts containing all grades of PanIN lesions was measured by examining 10 high-power fields (HPFs; 40×) per slide. PanINs were graded according to established criteria (Hruban et al., 2001, The American journal of surgical pathology 25, 579-586): In PanIN I ducts, the normal cuboidal pancreatic epithelial cells transition to columnar architecture (PanIN Ia) and gain polyploid morphology (PanIN Ib). PanIN II lesions are associated with loss of polarity. PanIN III lesions, or in-situ carcinoma, show cribriforming, budding off of cells, and luminal necrosis with marked cytological abnormalities, without invasion beyond the basement membrane. Slides were evaluated by an expert pancreas pathologist (CH). Immunohistochemistry (IHC) was performed using antibodies directed against CD4 (RM4-5; BD Bioscience) and CD8 (YTS169.4; Abcam). Quantifications were performed by assessing 10 HPF per slide. For immunofluorescent staining, frozen specimens were probed with antibodies directed against TCRγ/δ (GL3; Biolegend), TCRαβ (H57-597; Biolegend), or CD11b (M1/70; Biolegend). For analysis of human tissues, frozen sections of human pancreatic cancer specimens were probed with antibodies directed against TCRγ/6 (B1.1; eBioscience), TCRαβ (TP26; Biolegend), PD-L1 (Polyclonal, Abcam), or CD11b (M1/70; Biolegend). Images were acquired using the Zeiss LSM700 confocal microscope along with ZEN software (Carl Zeiss, Thornwood, N.Y.). The proximity of αβT cells to γδT cells or CD11b⁺ cells, respectively, was determined by measuring the distance between each αβT cell and its spatially closest counterpart. Distances were measured in micrometers on low power fields (20×). The averages distances were calculated for 10 low power fields per pancreas.

(VII) Intravital Imaging

Orthotopic pancreas tumor-bearing C57BL/6-Trdc^{tm1Mal} mice were anesthetized and a left subcostal laparotomy incision was made. The spleen and pancreatic tumor were externalized. The mouse was then placed prone on a heated (37° C.) stage mounted with a coverslip which was in contact with the pancreatic tumor. To visualize the pancreatic vasculature, mice were injected i.v. with 25 μg Evan Blue (Sigma) 10 min before imaging. Images were acquired with a LSM 710 inverted microscope (Zeiss) with a MaiTai Ti: Sapphire laser (Spectra-Physics, Santa Clara, Calif.) tuned to 910-930 nm. Emitted fluorescence was detected through 420/40, 465/30, 520/30, 575/70, and 660/50 nm band-pass filters and nondescanned detectors to generate second harmonic signals (collagen fibers) and 4-color images. All the images were acquired at least 50 μm below the tumor capsule. ZEN software was used for analysis.

(VIII) In Vitro T Cell Activation Assays

For T cell activation assays, spleen CD4⁺ or CD8⁺ T cells (5×10⁴) were labeled with CFSE (eBioscience) and plated alone or with PDA-infiltrating γδT cells, MDSC, or TAMs (5:1 ratio) in 96 well plates coated with anti-CD3 (145-2C11, 10 μg/ml) and anti-CD28 (37.51; 10 μg/ml, both Biolegend). After 72 hours, αβT cells were harvested and analyzed by flow cytometry. In selected experiments, cells were treated with a neutralizing mAb directed against PD-L1 (10F.9G2, 10 μg/ml; BioXCell).

### Results

(I) Activated γδT Cells are Ubiquitous in Human PDA

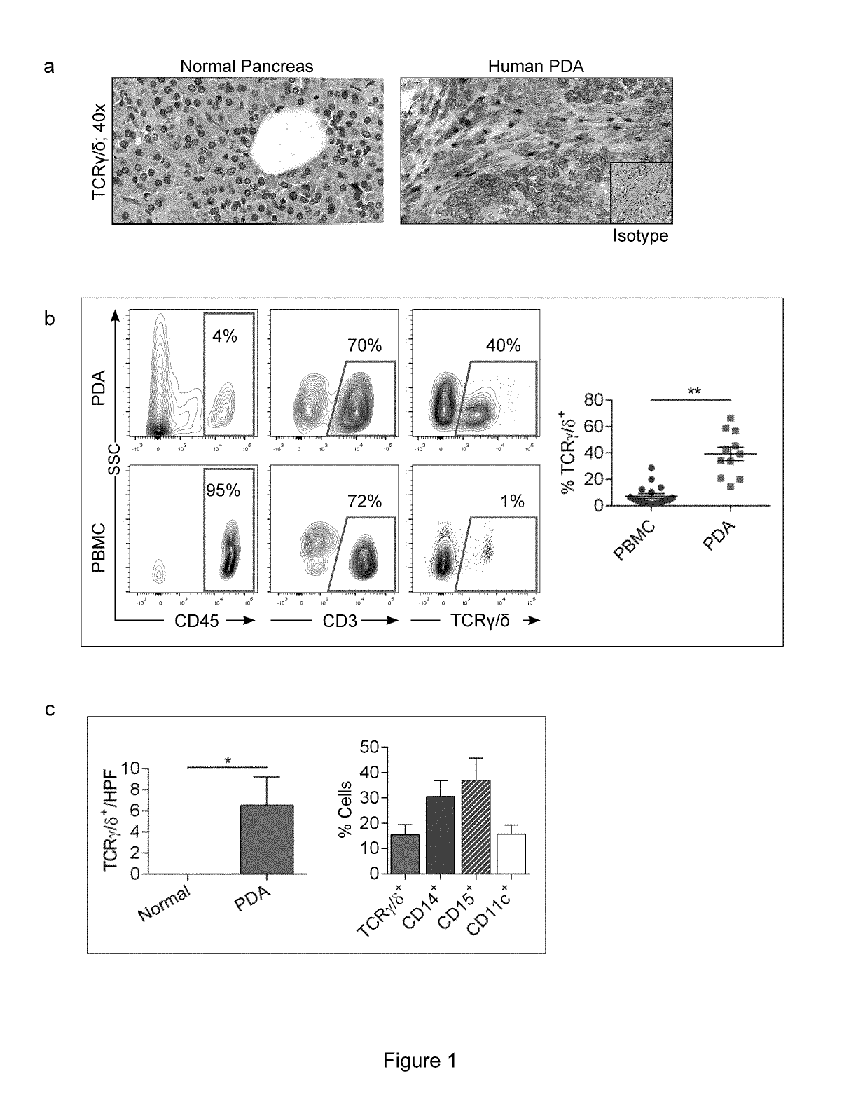

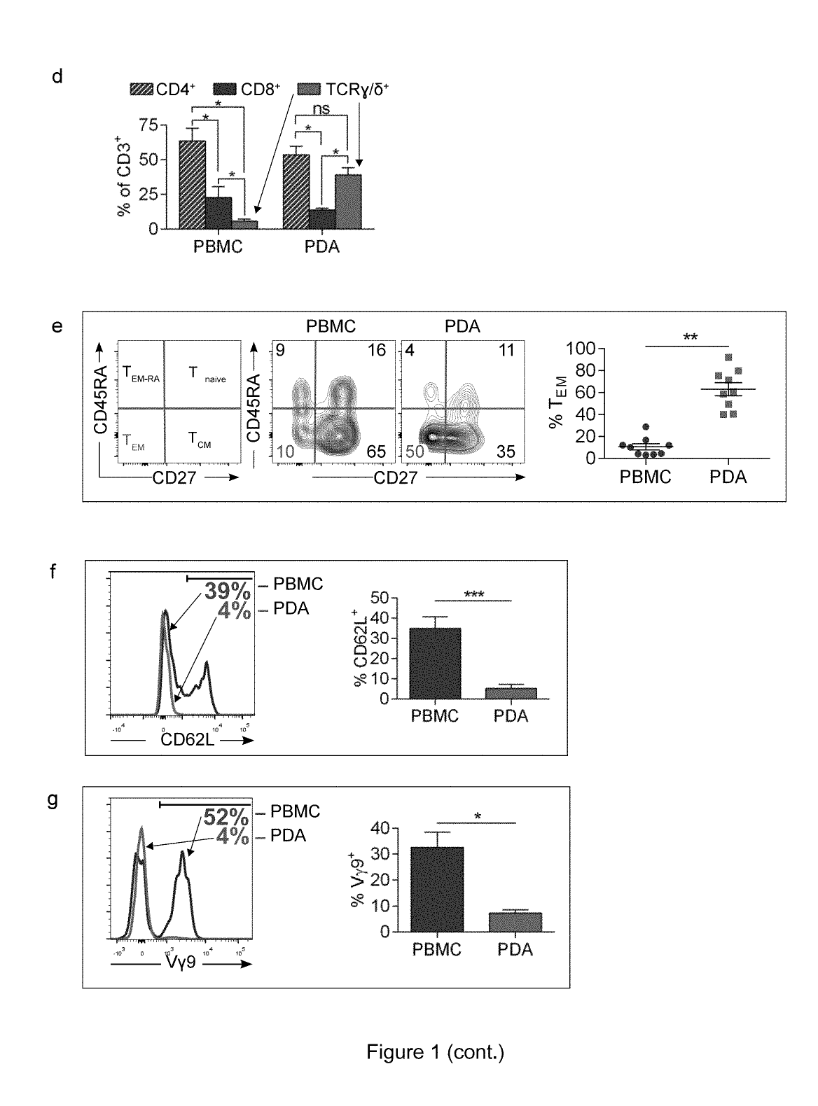

Immunohistochemical analysis revealed that γδT cells are widely distributed within the human PDA tumor stroma but absent in normal pancreas (FIG. 1a). Moreover, up to 75% of human PDA-infiltrating T cells were TCRγ/δ⁺ compared with a much lower fraction in PBMC (FIG. 1b). On average, γδT cells had a similar prevalence to select myeloid-derived cellular subsets within the PDA TME (FIG. 1c) and comprised a significantly higher percentage of tumor-infiltrating lymphocytes compared with CD8⁺ T cells (FIG. 1d). Human T cell subsets, including γδT cells, can be broadly classified as central memory (TCM) or effector memory (TEM) based on their co-expression of CD45RA and CD27 (Sallusto et al., 2004, Annual review of immunology 22, 745-763). We found γδT cells in PBMC were predominantly TCM whereas PDA-infiltrating γδT cells were mostly TEM cells, indicative of a distinctly activated phenotype (FIG. 1e). Accordingly, tumor-infiltrating γδT cells down-regulated CD62L compared with their counterparts in PBMC (FIG. 1f). However, Vγ9⁺ γδT cells associated with tumoricidal function (Izumi et al., 2013, Cytotherapy 15, 481-491; Kunzmann et al., 2012, Journal of immunotherapy 35, 205-213) were notably absent in PDA, suggestive of tumor-permissive properties (FIG. 1g).

(II) A Distinctly Activated γδT Cell Population is Prominent in Invasive and Pre-Invasive Murine PDA

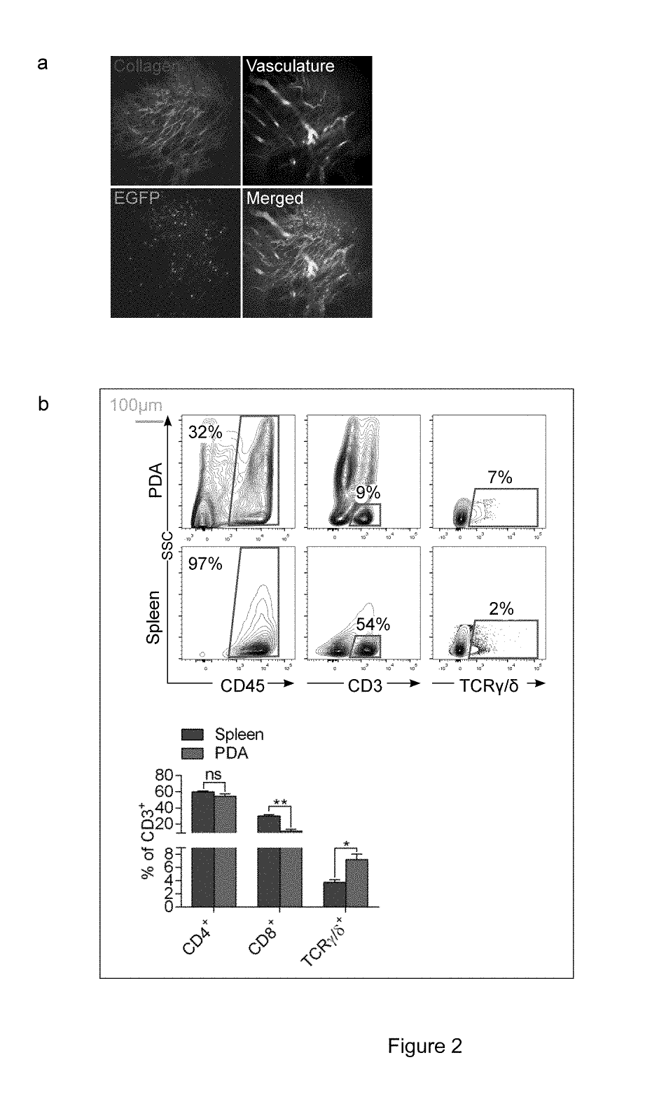

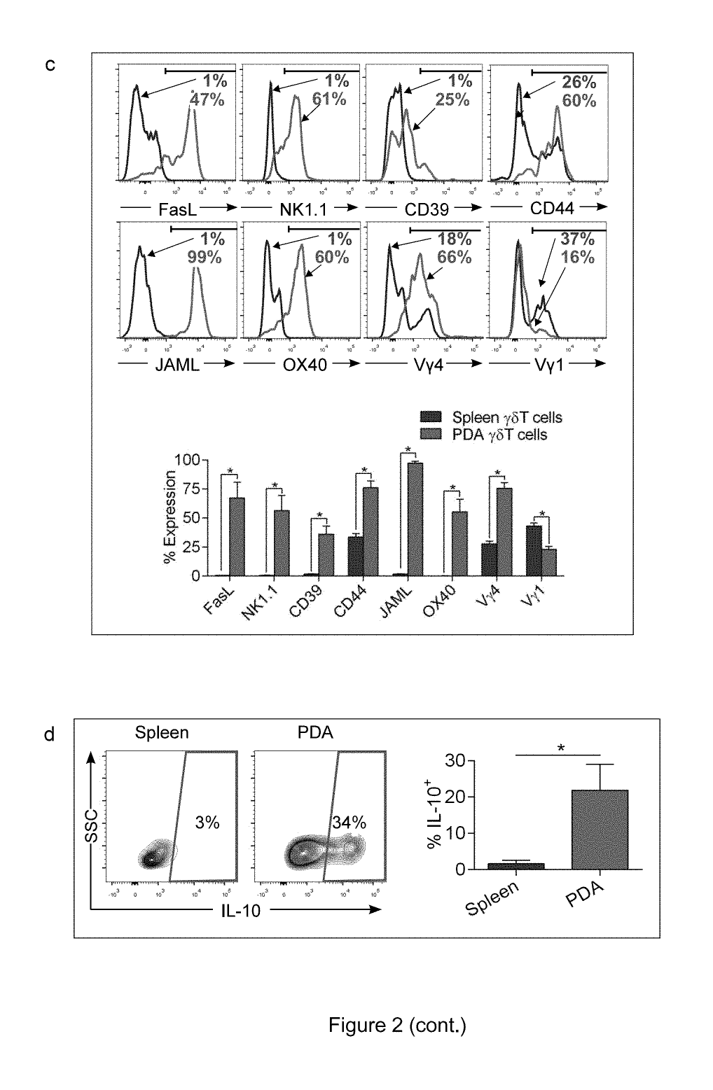

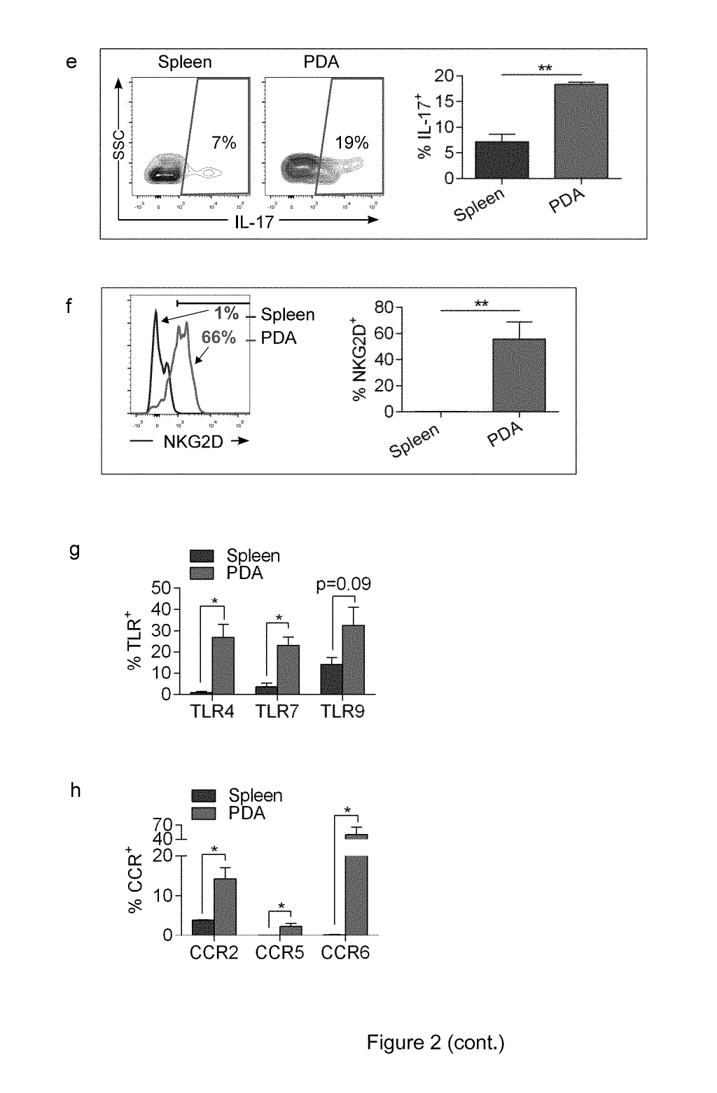

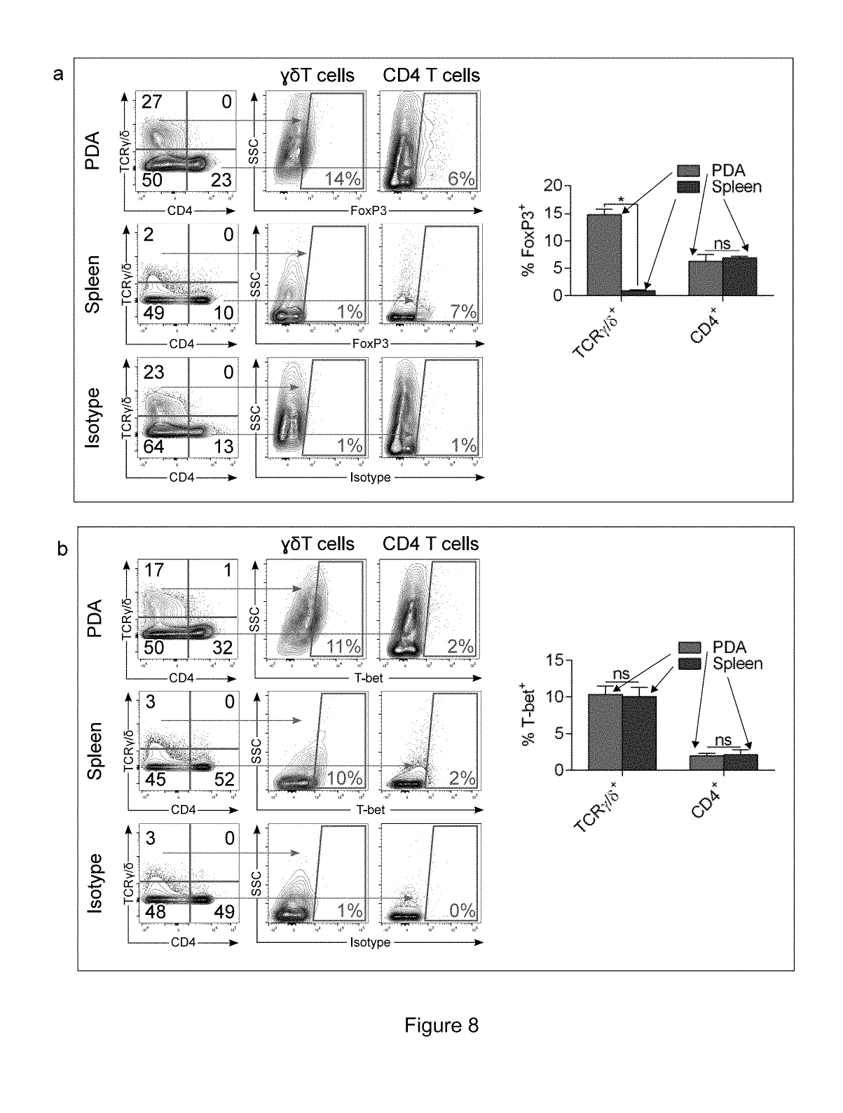

In vivo imaging of pancreata from C57BL/6-Trdc^{tm1Mal} mice harboring orthotopically implanted Pdx1^{Cre};Kras^{G12D};Tp53^{R172H} (KPC)-derived invasive PDA suggested that γδT cells were highly prevalent in the interstitial space of murine PDA (FIG. 2a). Flow cytometry suggested a higher frequency of γδT cells infiltrating orthotopic KPC tumors compared with the spleen of tumor-bearing mice (FIG. 2b). Similar to human disease, the population of PDA-infiltrating γδT cells in mice were distinctly activated expressing higher FasL, NK1.1, CD39, CD44, JAML, and OX40 compared with spleen γδT cells (FIG. 2c). Further, in contrast to spleen, PDA-infiltrating γδT cells contained a prominent Vγ4⁺ subset whereas Vγ1⁺ cells were rare (FIG. 2c). Tumor-infiltrating γδT cells also expressed elevated levels of IL-10 and IL-17 (FIG. 2d, e). Similarly, Th1-(TNFα, IFNγ), and additional Th2-(IL-13) cytokines were highly expressed in PDA-infiltrating γδT cells (not shown). Moreover, PDA-infiltrating γδT cells exhibited a substantial FoxP3⁺ fraction which has been associated with immune suppressive function (Kang et al., 2009, Immunology letters 125, 105-113), compared with absent expression of FoxP3⁺ in spleen γδT cells (FIG. 8a). Conversely, T-bet was equally expressed in γδT cells in both compartments (FIG. 8b). Further, PDA-infiltrating γδT cells expressed high levels of the NKG2D receptor (FIG. 2f) as well as elevated TLR4, TLR7 and TLR9 (FIG. 2g) which are potential avenues for cellular activation in PDA (Zambirinis et al., 2015, The Journal of experimental medicine 212, 2077-2094). CCR2, CCR5, and CCR6 were also upregulated in PDA-infiltrating γδT cells (FIG. 2h).

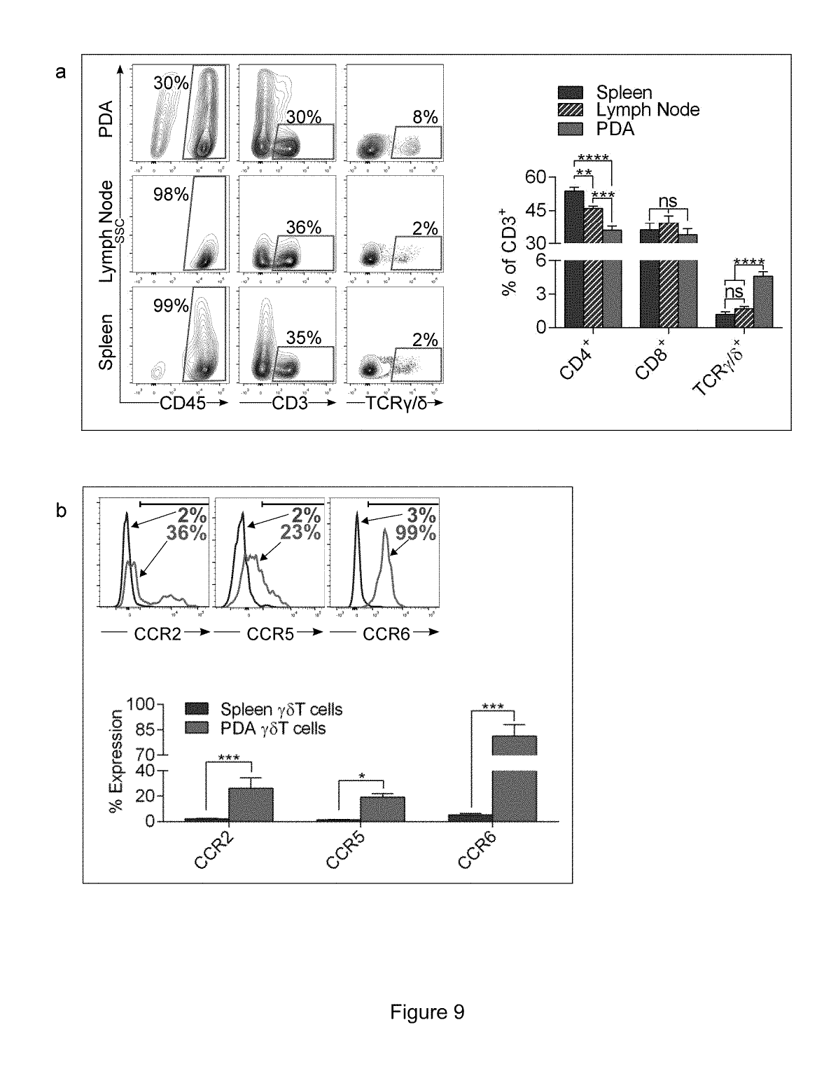

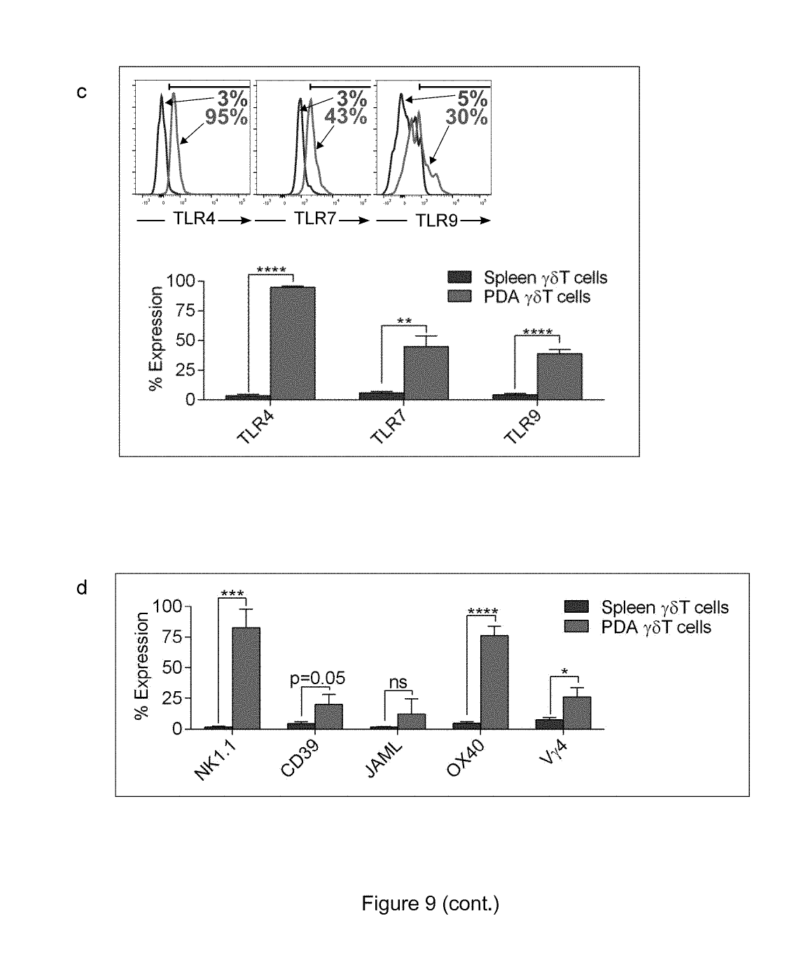

To determine whether γδT cells were similarly prominent in a slowly progressive model of PDA, we interrogated pancreata of 6 month-old p48^{Cre};Kras^{G12D} (KC) mice harboring pre-invasive tumor. γδT cells represented ˜6-8% of CD3⁺ T cells in the pancreas of KC mice compared with ˜2% in the spleen and tumor-draining lymph nodes (FIG. 9a). Further, similar to mice with invasive PDA, γδT cells expressed high levels of chemokine receptors (FIG. 9b), TLRs (FIG. 9c), and activation markers, and included a prominent Vγ4⁺ fraction (FIG. 9d).

(III) γδT Cell Recruitment and Activation in PDA is Contingent on Diverse Chemokine Signaling

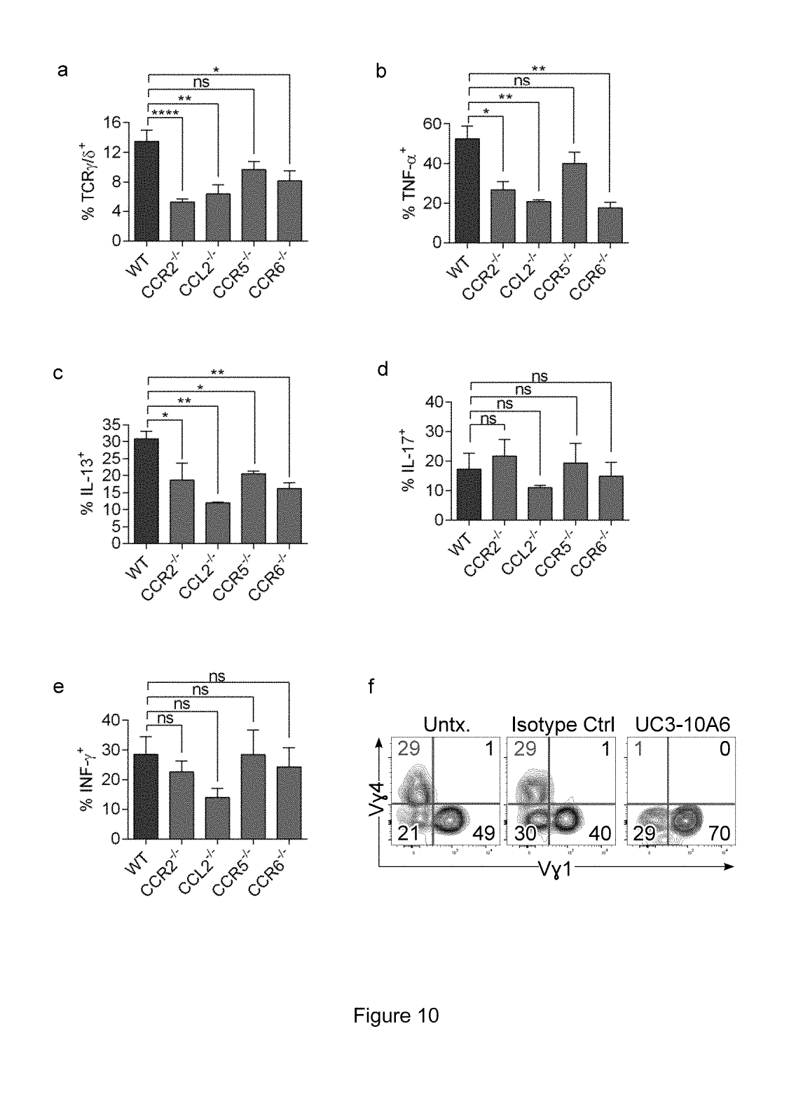

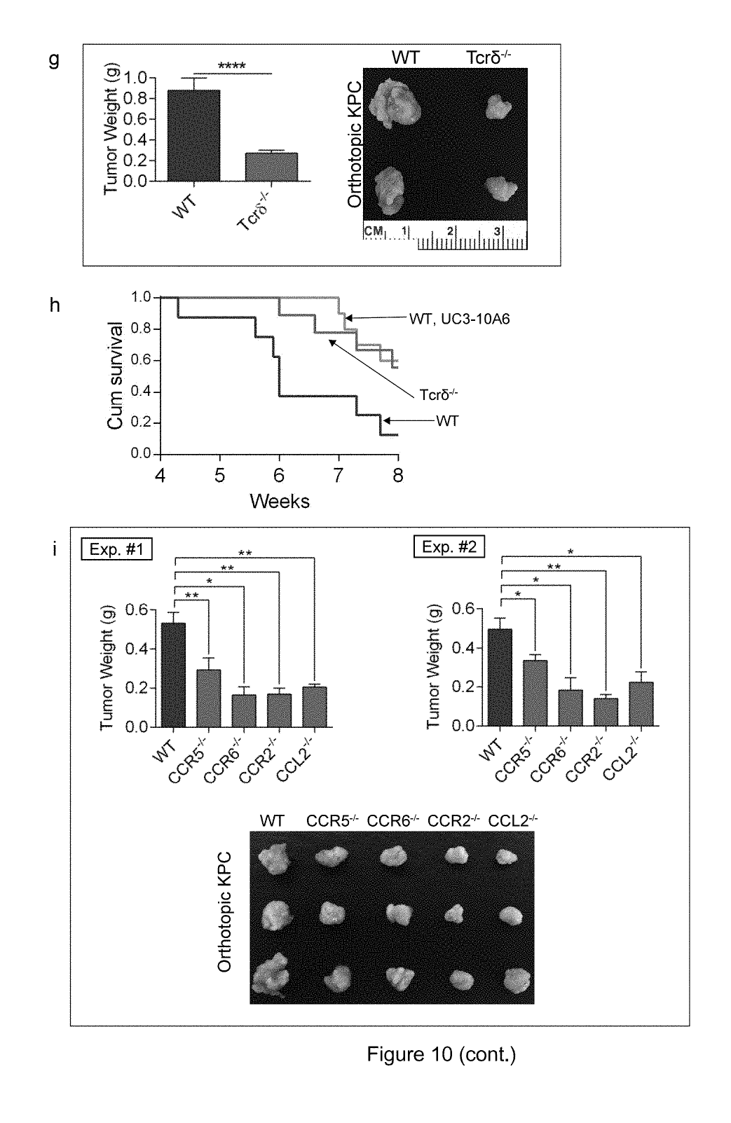

Since we found that PDA-infiltrating γδT cells express high CCR2, CCR5, and CCR6, we postulated that ligation of these receptors is critical in their recruitment to the TME. To test this, we challenged CCR2^{-/-}, CCL2^{-/-}, CCR5^{-/-}, and CCR6^{-/-} mice with orthotopic KPC-derived tumor and measured γδT cell infiltration on day 21. Deletion of CCR2, CCL2, or CCR6 significantly reduced γδT cell infiltration to the TME (FIG. 10a). Moreover, selective CCR2, CCR5, CCR6 or CCL2 deletion mitigated TNF-α and IL-13 expression from PDA-infiltrating γδT cells whereas γδT cell expression of IL-17 and IFN-γ were not affected (FIG. 10b-e). γδT cell expression of FoxP3 or IL-10 were similarly not perturbed by blockade of chemokine signaling (not shown).

(IV) γδT Cells Promote Pancreatic Oncogenesis

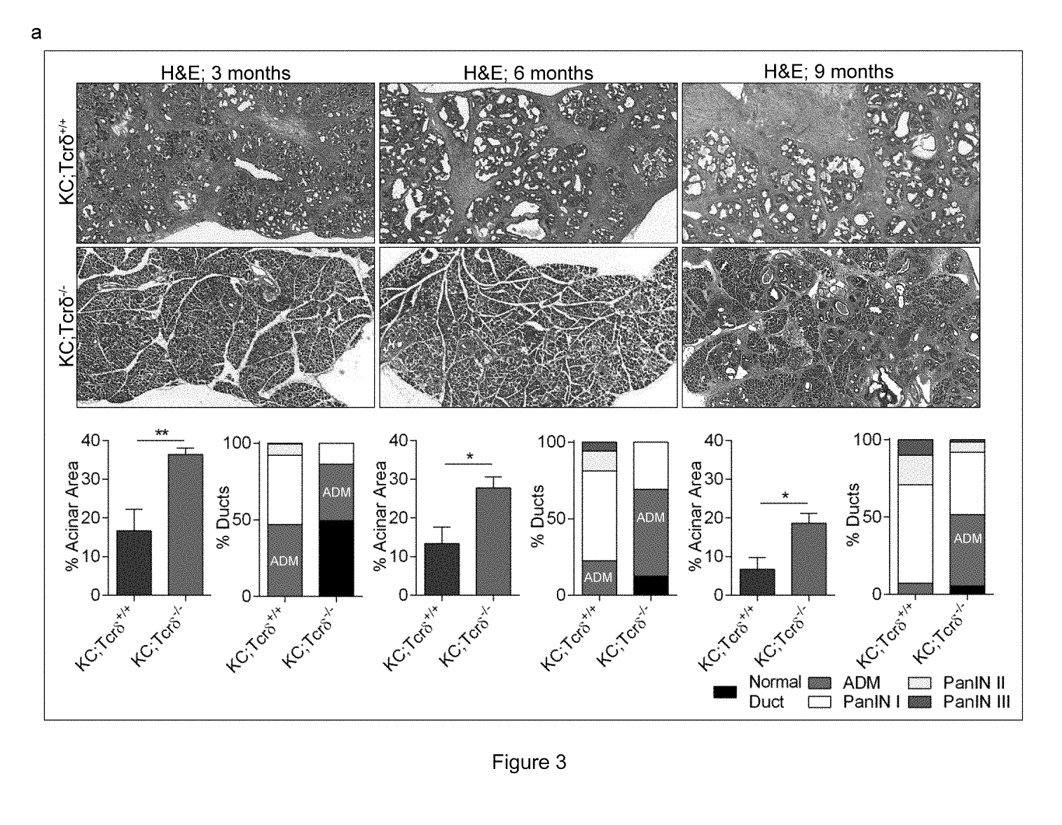

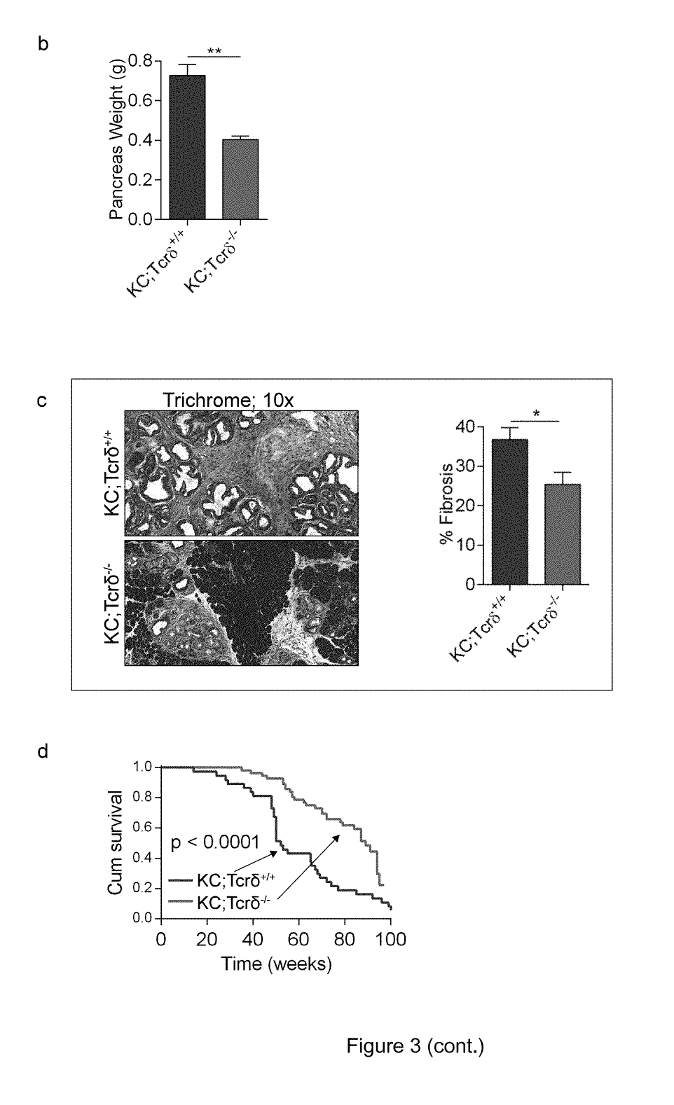

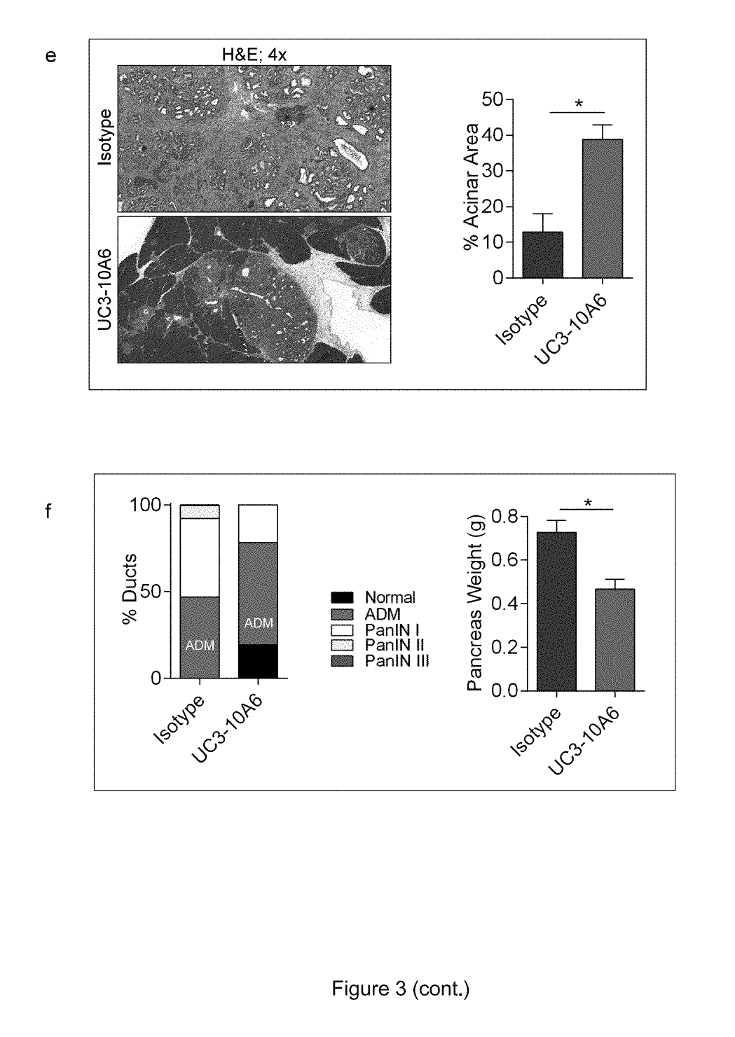

Since γδT cells are a prominent lymphocytic subset within the pancreatic TME, we postulated that they play a critical role in oncogenesis. To test this, we crossed KC mice with Tcrδ^{-/-} mice. Pancreata of KC;Tcrδ^{-/-} mice were protected from progressive oncogenesis exhibiting a diminished rate of acinar replacement by dysplastic ducts and substantially slower PanIN progression at multiple time-points (FIG. 3a). Analysis of pancreas weights confirmed the protective effects of γδT cell deletion (FIG. 3b). γδT cell ablation was also associated with reduced peri-tumoral fibrosis (FIG. 3c). Moreover, Kaplan-Meier analysis revealed a nearly 1 year increase in the median survival of γδT cell-deficient KC mice compared with controls (FIG. 3d).

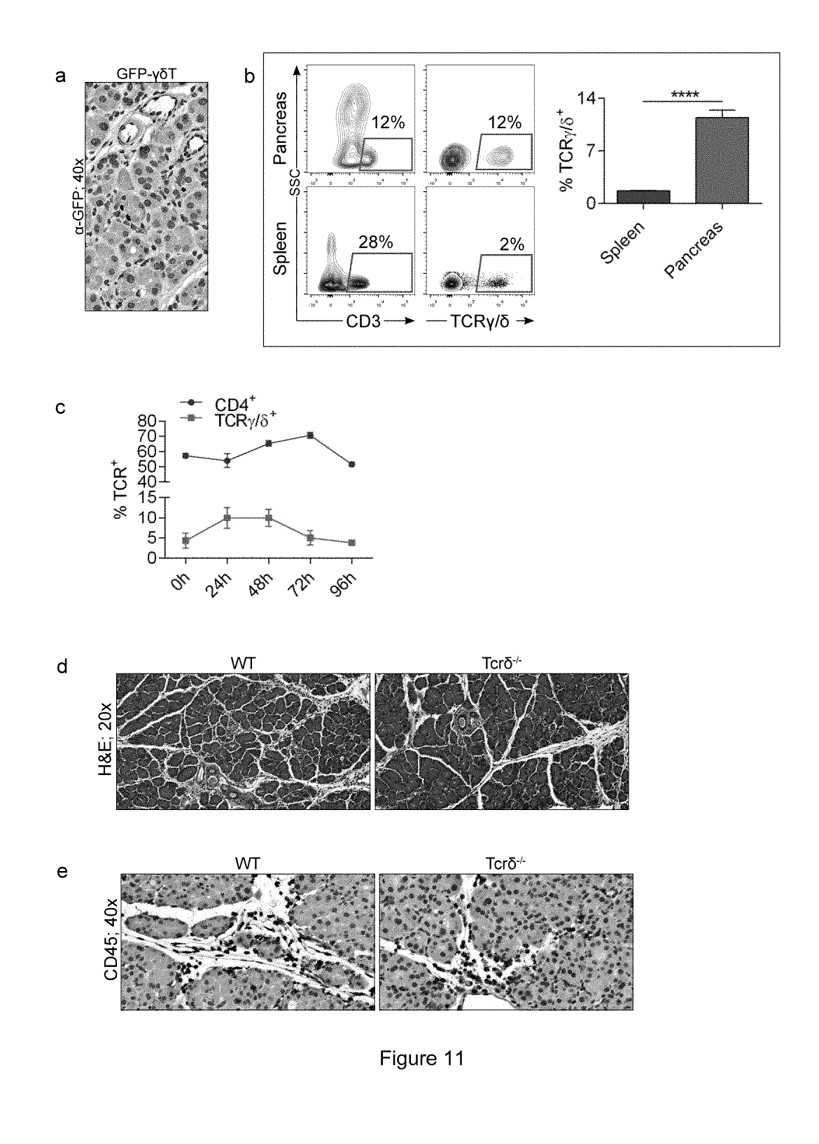

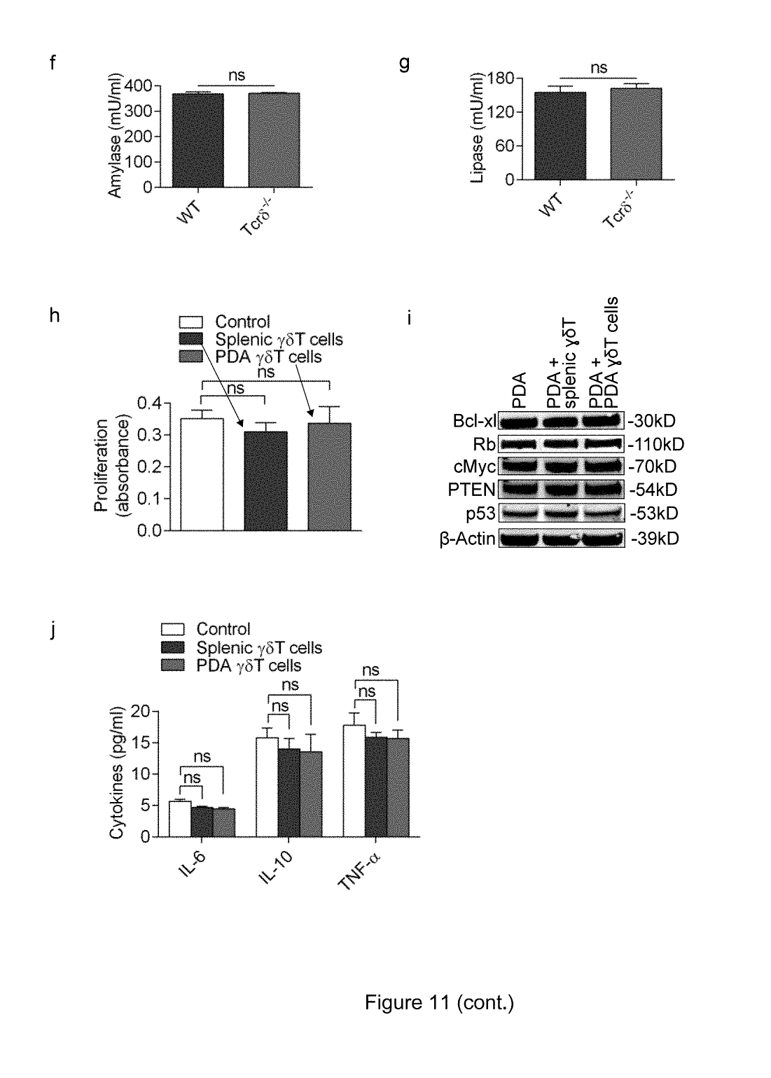

Since genetic deletion of γδT cells has limited translational applicability to human disease, we tested whether in vivo depletion of Vγ4⁺γδT cells using a neutralizing mAb would offer similar protection (FIG. 10f). We treated 6 week-old KC mice for 8 weeks with UC3-10A6 or isotype control and assessed their effects on tumorigenesis. γδT cell depletion protected against oncogenic progression based on histological analysis of ductal transformation (FIG. 3e) and tumor mass (FIG. 3f). To determine whether the presence of γδT cells are similarly associated with accelerated tumorigenesis in an invasive model of PDA, we orthotopically implanted KPC-derived tumor cells into the pancreatic body of WT and Tcrδ^{-/-} mice. Consistent with our data in KC mice, deletion of γδT cells impressively protected against tumor growth and extended survival in the orthotopic KPC model (FIGS. 10g, h). γδT cell depletion similarly extended survival in invasive PDA (FIG. 10g). Moreover, blocking γδT cell recruitment and activation using mice deficient in selective chemokine signaling was also protective (FIG. 10i). Notably, disease phenotype in caerulein-induced pancreatitis was not mitigated in Tcrδ^{-/-} mice suggesting that the ability of γδT cells to modulate pancreatic disease is specific to PDA (FIG. 11a-g).

(V) PDA-Infiltrating γδT Cells do not have Direct Pro-Tumorigenic Effects on Epithelial Cells

We hypothesized that γδT cells may have direct oncogenic effects on transformed epithelial cells. To test this, we co-cultured tumor cells derived from KPC mice with FACS-sorted PDA-infiltrating γδT cells. However, γδT cells failed to enhance proliferation (FIG. 11h) or deregulate expression of oncogenic or tumor suppressor genes (FIG. 11i) in transformed epithelial cells. Similarly, γδT cell co-culture did not elicit pro-inflammatory or regulatory cytokine production from tumor cells suggesting that PDA-infiltrating γδT cells do not promote tumorigenesis via direct engagement of cancer cells (FIG. 11j).

(VI) γδT Cells Support an Immune Suppressive Pancreas Tumor Microenvironment in Invasive and Pre-Invasive PDA

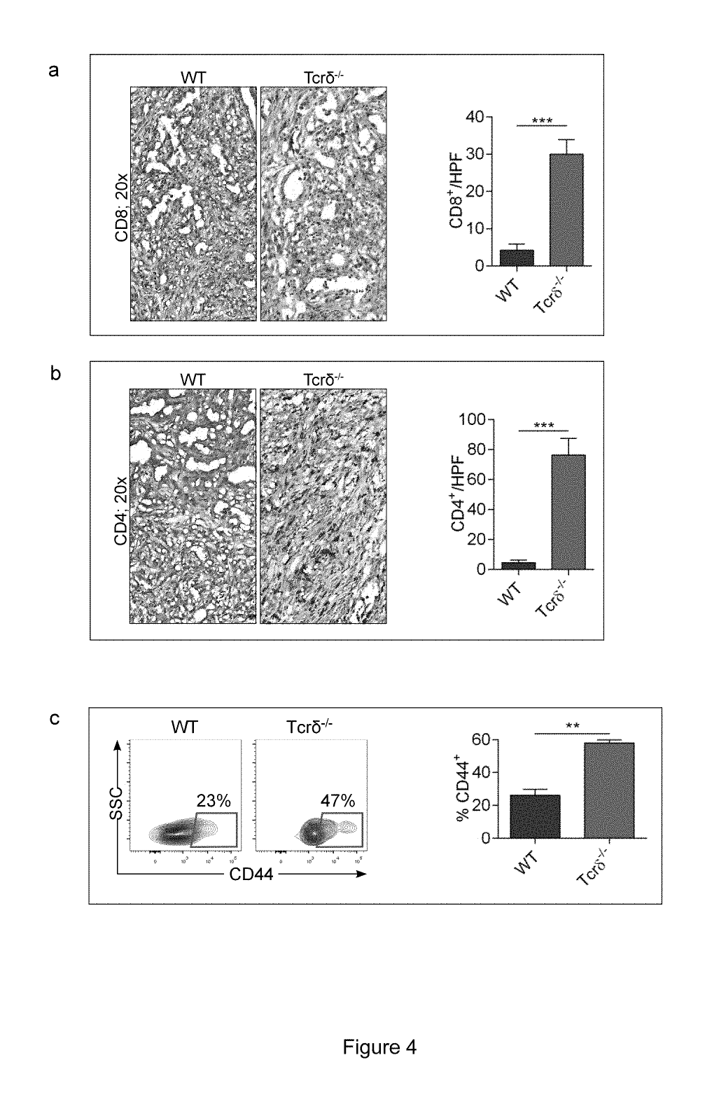

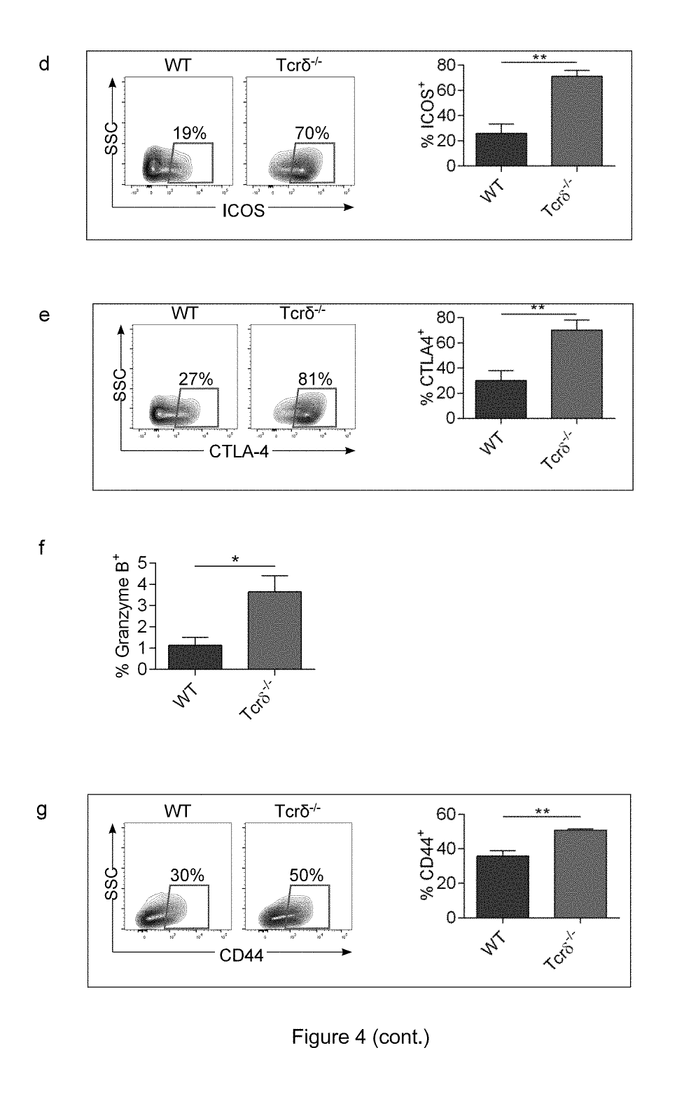

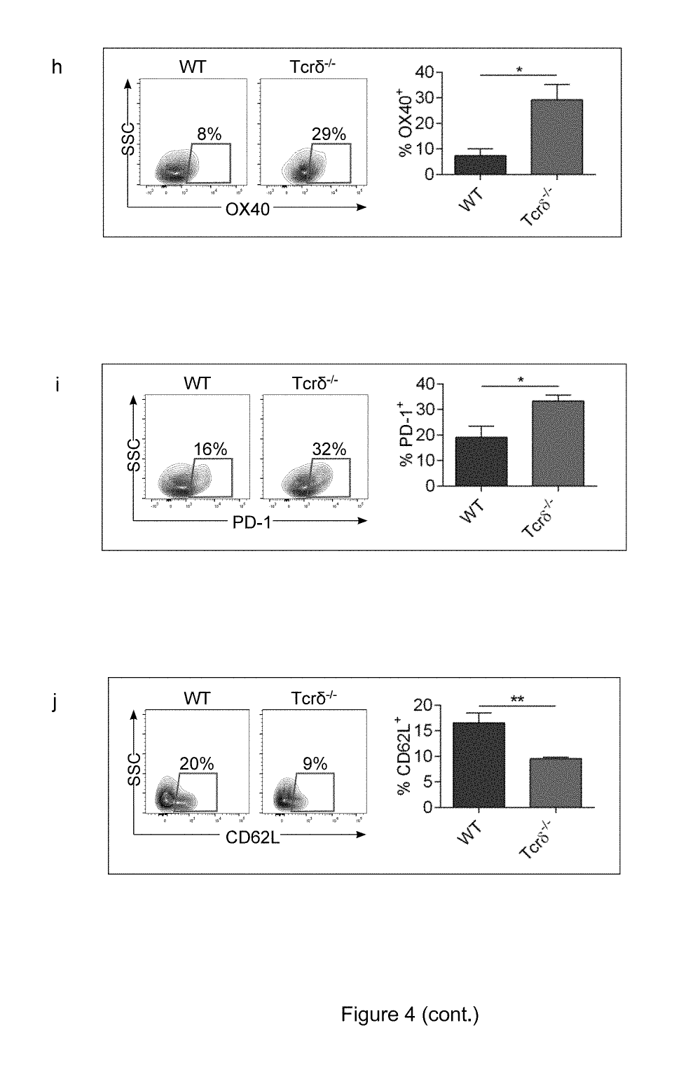

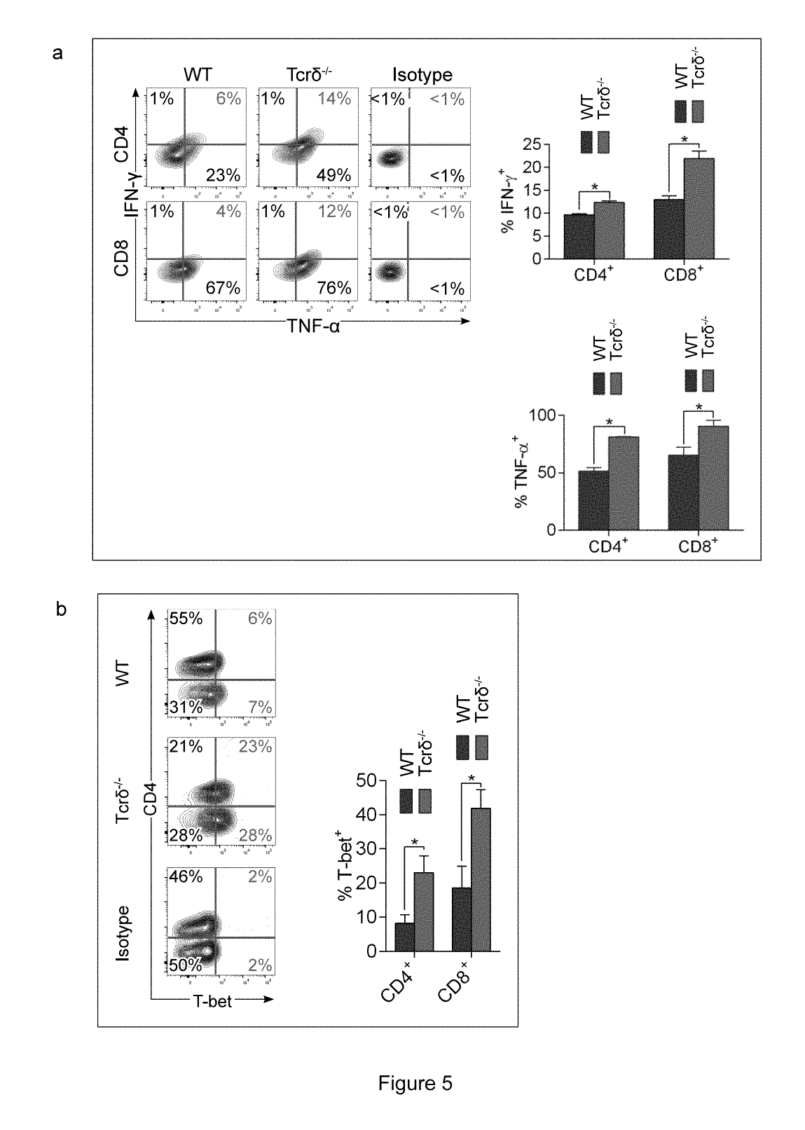

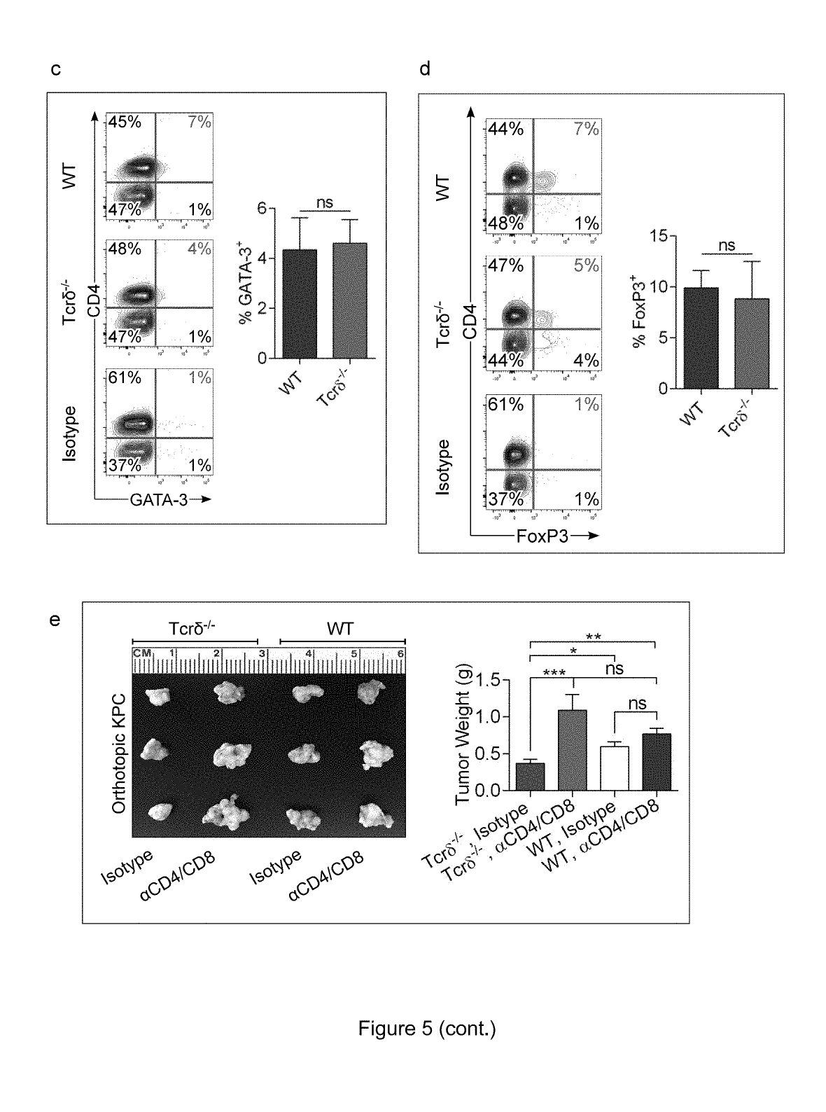

Intra-pancreatic γδT cells may promote tumorigenesis by engendering an immune-suppressive pancreatic TME. It was found in this study that whereas CD4⁺ and CD8⁺ T cells were scarce in invasive PDA tumors, tumor-infiltrating CD4⁺ and CD8⁺ T cells increased ˜10-fold in absence of γδT cells (FIG. 4a, b). Moreover, besides expanding in number, PDA-infiltrating αβT cells were markedly activated in Tcrδ^{-/-} hosts. CD8⁺ T cells infiltrating γδT cell-deficient tumors expressed higher CD44 (FIG. 4c), ICOS (FIG. 4d), CTLA4 (FIG. 4e), and Granzyme B (FIG. 4f), each indicative of higher cytotoxic T cell activation. Similarly, CD4⁺ T cells infiltrating γδT cell-deficient tumors expressed higher CD44 (FIG. 4g), OX40 (FIG. 4h), and PD-1 (FIG. 4i), and lower CD62L (FIG. 4j). Further, both CD4⁺ and CD8⁺ T cells expressed elevated TNF-α and IFN-γ in γδT cell-deleted tumors, indicative of enhanced Th1-differentation and higher CD8⁺ T cell cytotoxicity (FIG. 5a). Accordingly, PDA-infiltrating CD4⁺ and CD8⁺ T cells each sharply upregulated T-bet expression in the context of γδT cell deletion (FIG. 5b). GATA-3 and FoxP3 expression in CD4⁺ T cells were not affected by γδT cell deletion (FIG. 5c, d). Collectively, these data suggest immunogenic reprogramming of adaptive αβT lymphocytes in PDA in the absence of γδT cells.

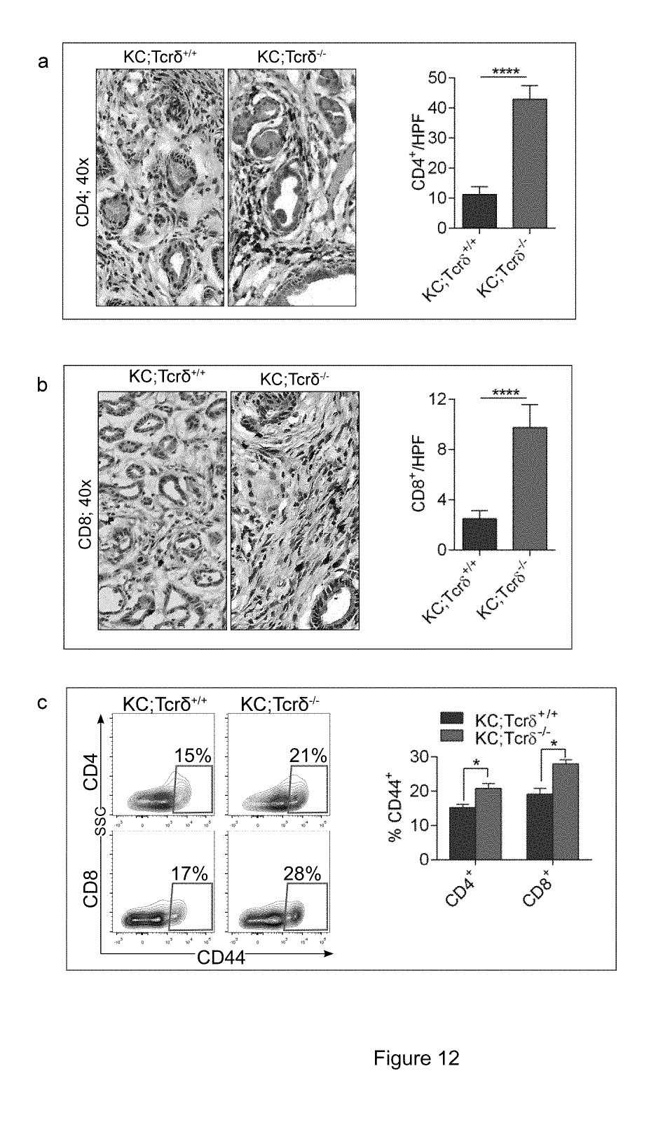

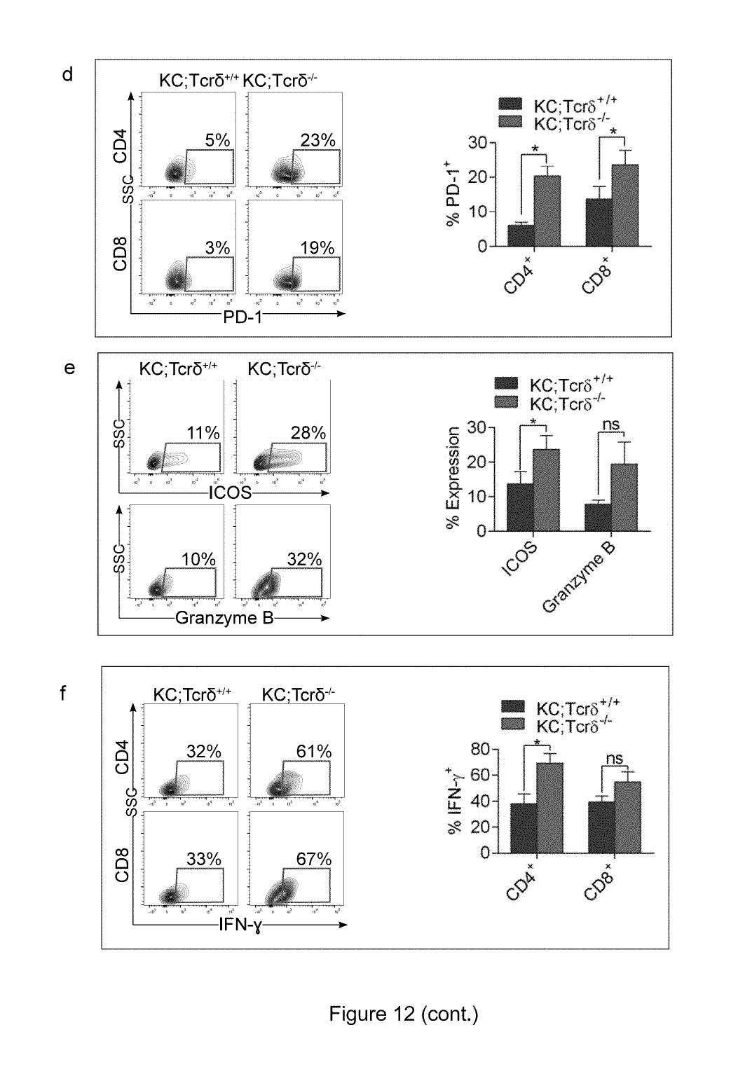

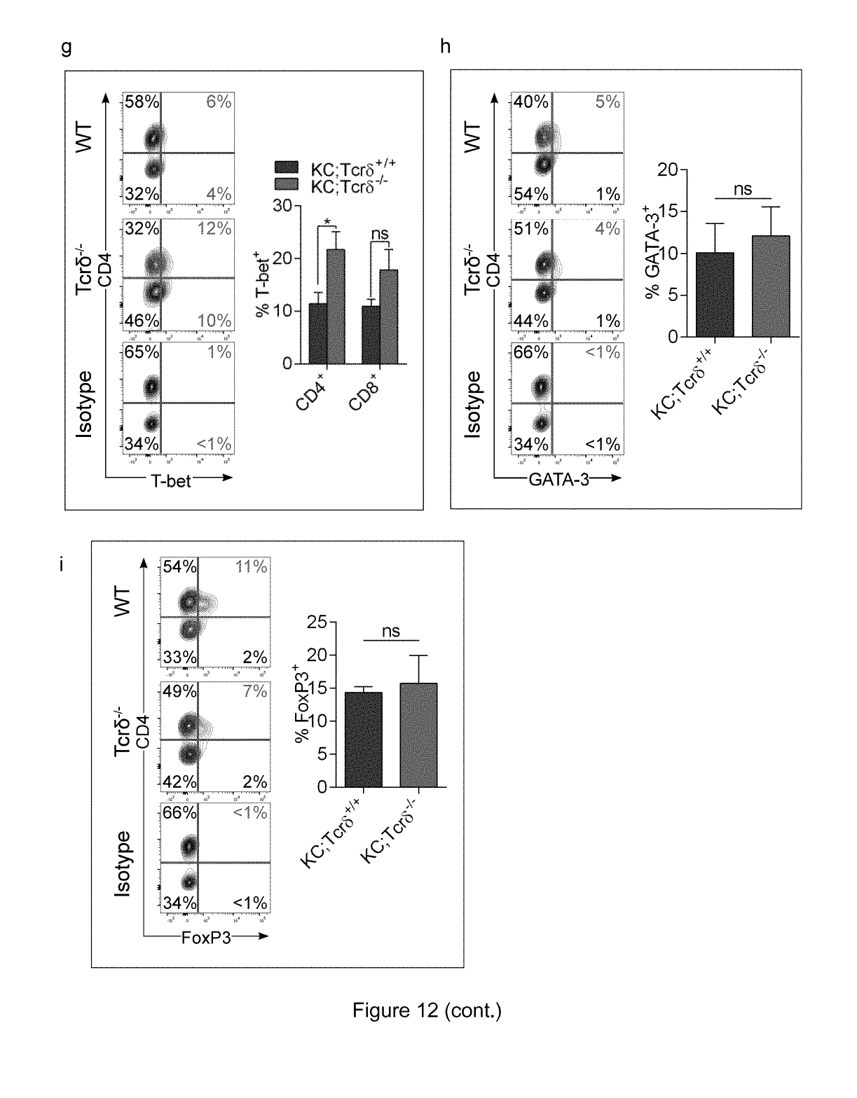

To determine whether γδT cells similarly delimit αβT cell expansion and activation in a slowly progressive model of PDA, we compared CD4⁺ and CD8⁺ T cell phenotype in KC;Tcrδ^{+/+} versus KC;Tcrδ^{-/-} pancreata. We found that while CD4⁺ and CD8⁺ T cells were scarce in KC;Tcrδ^{+/+} controls, both lymphocyte populations were markedly expanded in KC;Tcrδ^{-/-} pancreata (FIG. 12a, b). Further, both CD4⁺ and CD8⁺ T cells in PDA-draining lymph nodes expressed higher CD44 (FIG. 12c) and PD-1 (FIG. 12d) in KC;Tcrδ^{-/-} animals compared with KC;Tcrδ^{+/+}. Similarly, ICOS and Granzyme B expression were increased in CD8⁺ T cells in KC;Tcrδ^{-/-} hosts (FIG. 12e). Moreover, similar to the orthotopic KPC model, pancreas-draining CD4⁺ and CD8⁺ T cells in KC mice upregulated IFN-γ (FIG. 12f) and T-bet (FIG. 12g) in the context of γδT cell deletion whereas CD4⁺ T cell expression of GATA-3 and FoxP3 were unaffected by γδT cell deletion (FIG. 12h, i).

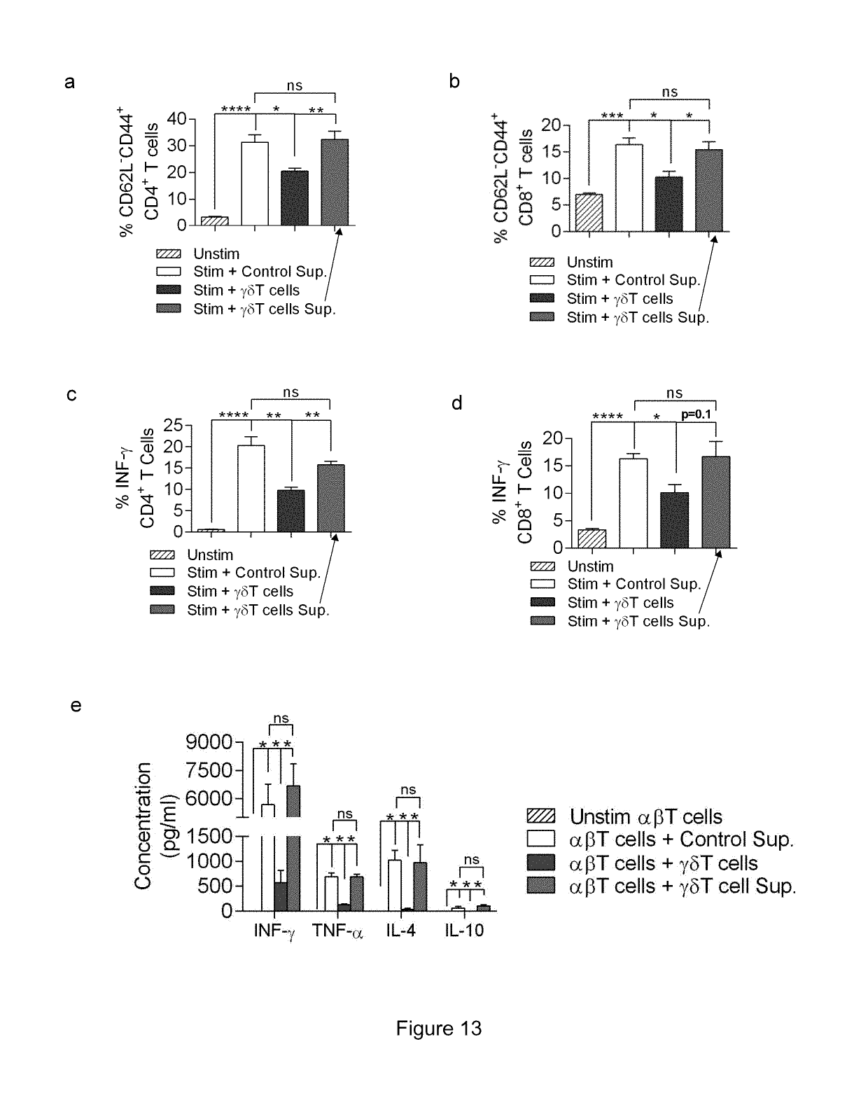

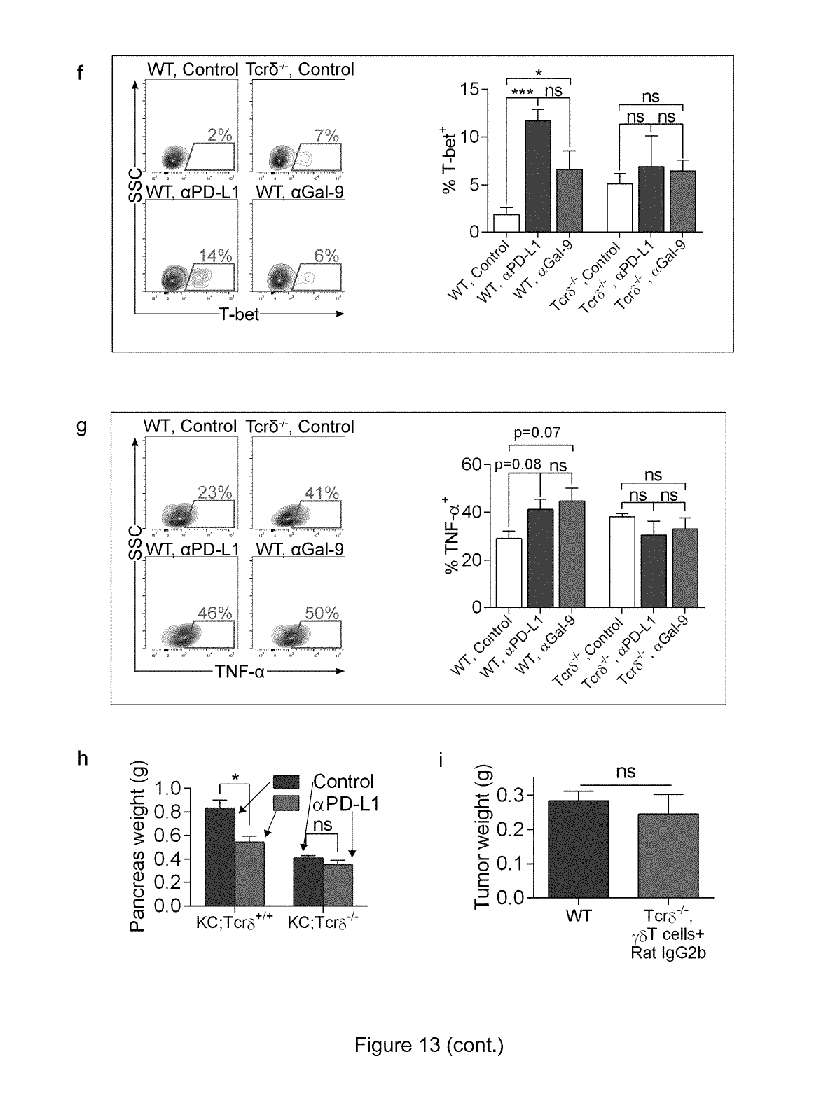

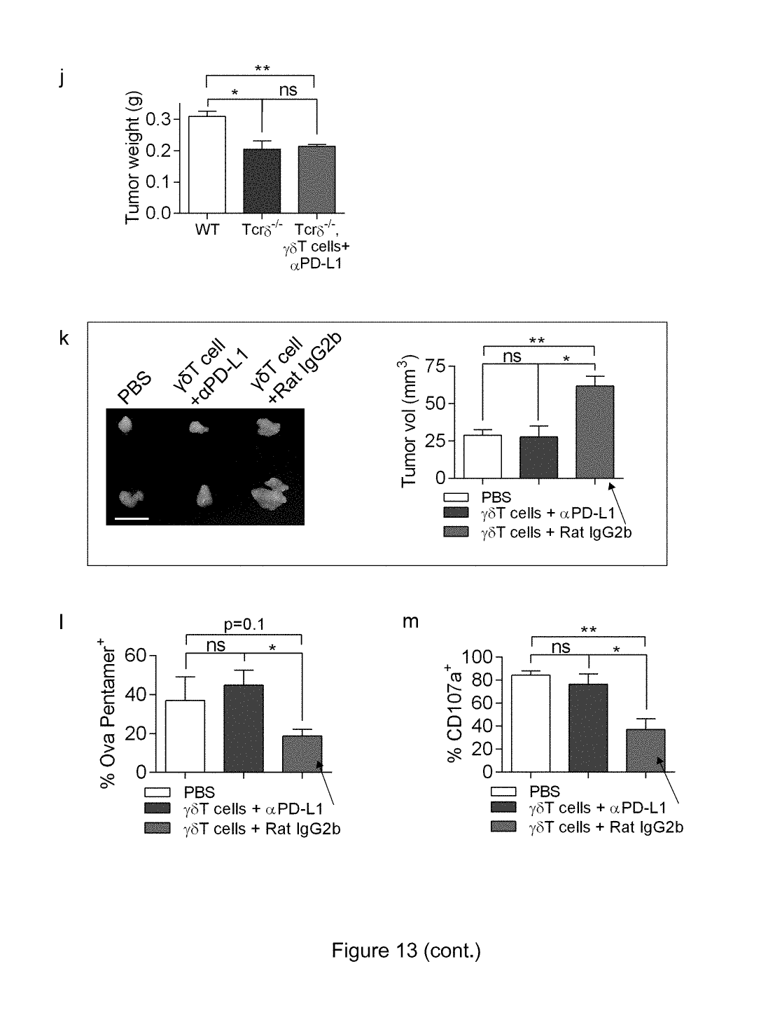

To definitively test whether enhanced αβT cell immunogenicity accounts for the protection against PDA observed in γδT cell-deficient animals, we depleted CD4⁺ and CD8⁺ T cells in Tcrδ^{-/-} mice and WT controls coincident with KPC-derived orthotopic tumor challenge. Ablation of αβT cell populations did not accelerate tumor growth in WT hosts but completely reversed the tumor-protective effects of γδT cell deletion. These data suggest that tumor-protection in PDA-bearing Tcrδ^{-/-} mice is mediated by αβT cells (FIG. 5e). To test whether PDA-infiltrating γδT cell inhibition of CD4⁺ and CD8⁺ T cells requires direct cellular interaction, we activated spleen αβT cells in vitro using CD3/CD28 co-ligation alone or in the context of either co-culture with PDA-derived γδT cells or admixture with γδT cell-conditioned media. Direct γδT cell coculture prevented CD4⁺ and CD8⁺ T cells from adopting an activated CD44⁺CD62L⁻ phenotype (FIG. 13a, b) and expressing immune-modulatory cytokines (FIG. 13c-e); however, γδT cell-conditioned media was non-inhibitory. These data suggest that γδT cells do not inhibit αβT cells via secreted factors but require direct cellular interaction.

(VII) Pancreas-Infiltrating γδT Cells Express High T Cell Exhaustion Ligands

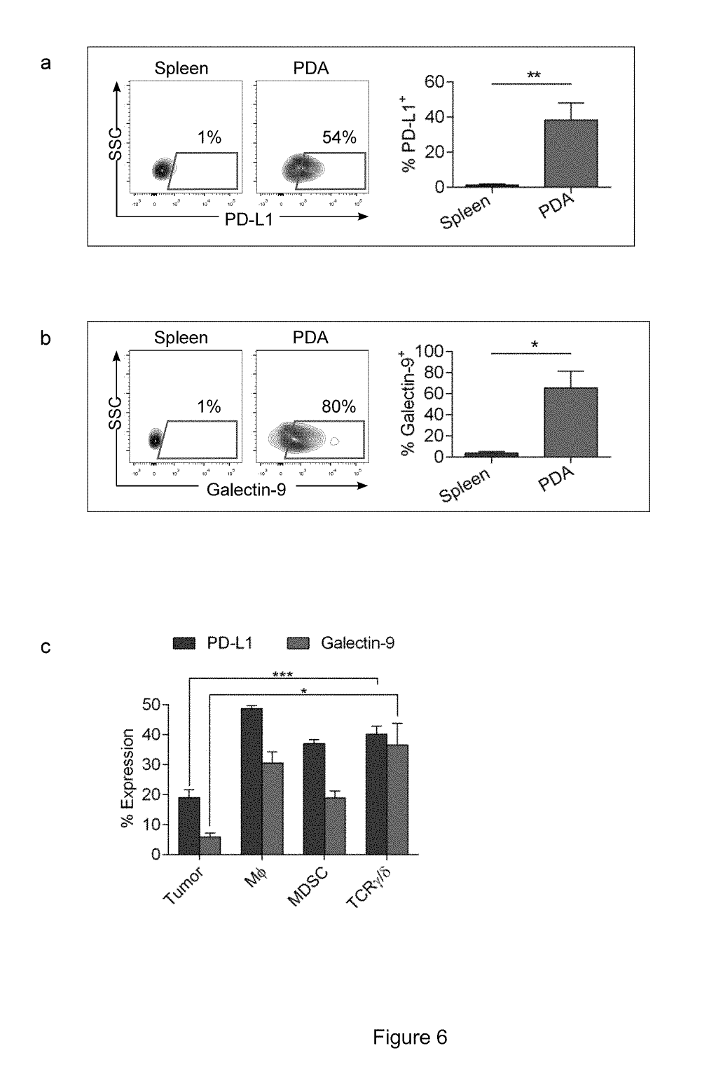

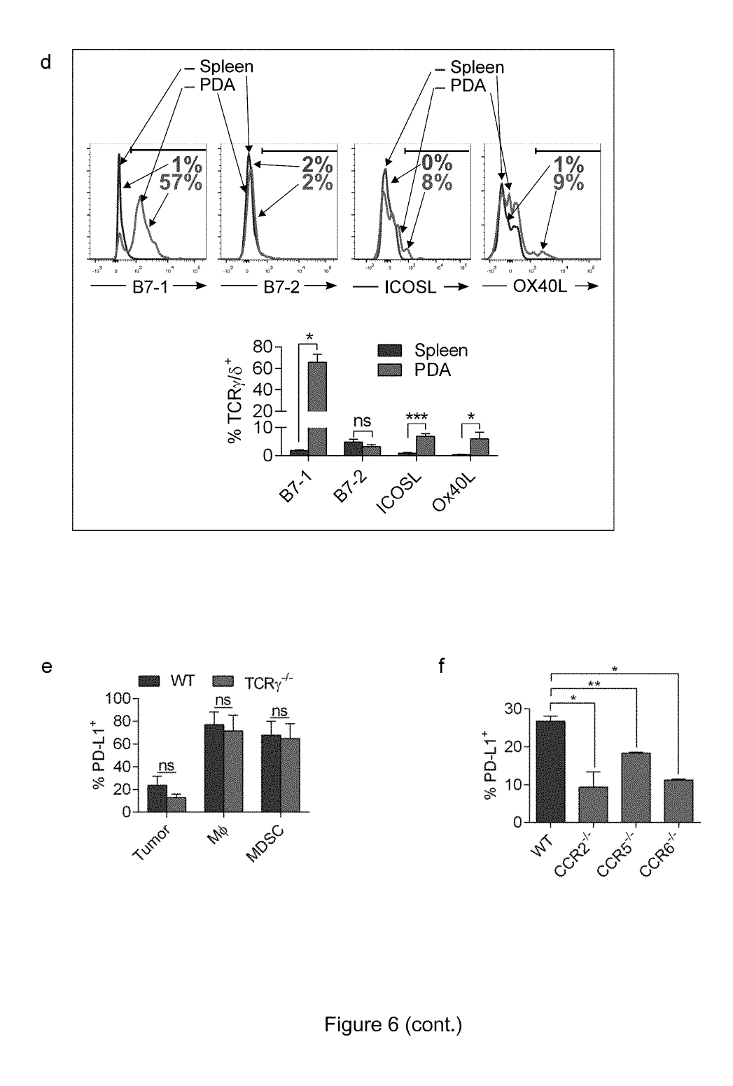

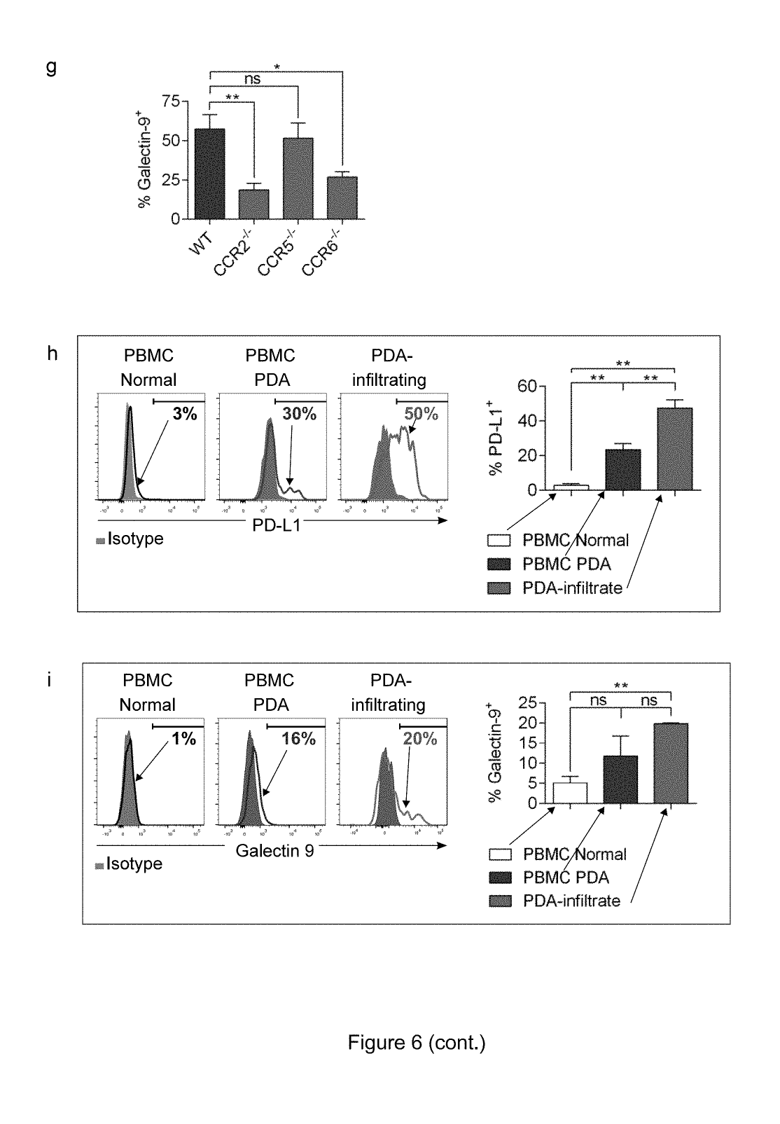

γδT cells may directly inhibit CD4⁺ and CD8⁺ T cell activation. It was discovered herein that PDA-infiltrating γδT cells in KC mice expressed high PD-L1 (FIG. 6a) and Galectin-9 (FIG. 6b) compared with absent expression of these ligands in spleen γδT cells. Similarly, γδT cells in orthotopic KPC tumors also expressed elevated PD-L1 and Galectin-9 (FIG. 6c). Expression levels of PD-L1 and Galectin-9 in PDA-infiltrating γδT cells were markedly higher than in cancer cells and comparable with that of tumor-infiltrating myeloid cell populations (FIG. 6c). By contrast, PDA-infiltrating γδT cells expressed elevated B7-1 but low levels of other activating ligands including B7-2, ICOSL, and OX40L in orthotopic KPC (FIG. 6d) and KC (not shown) tumors. Exhaustion ligand expression in myeloid or tumor cells in PDA was not affected by γδT cell deletion (FIG. 6e). Notably, besides regulating γδT cell expansion and activation, CCR2, CCR5, and CCR6 signaling were necessary for γδT cell expression of PD-L1 or Galectin-9 (FIG. 6f, g). To determine whether these findings translated to human disease, we tested PD-L1 expression in human PDA. Remarkably, PBMC γδT cells in PDA patients expressed elevated PD-L1 compared with absent PD-L1 expression in PBMC γδT cells from healthy subjects (FIG. 6h). Moreover, PD-L1 was expressed in 50% of tumor-infiltrating γδT cells in human PDA (FIG. 6i). Similarly, Galectin-9 was upregulated in human PDA-infiltrating γδT cells (FIG. 6j).

(VIII) γδT Cells Inhibit of Cell Activation Via Checkpoint Receptor Ligation

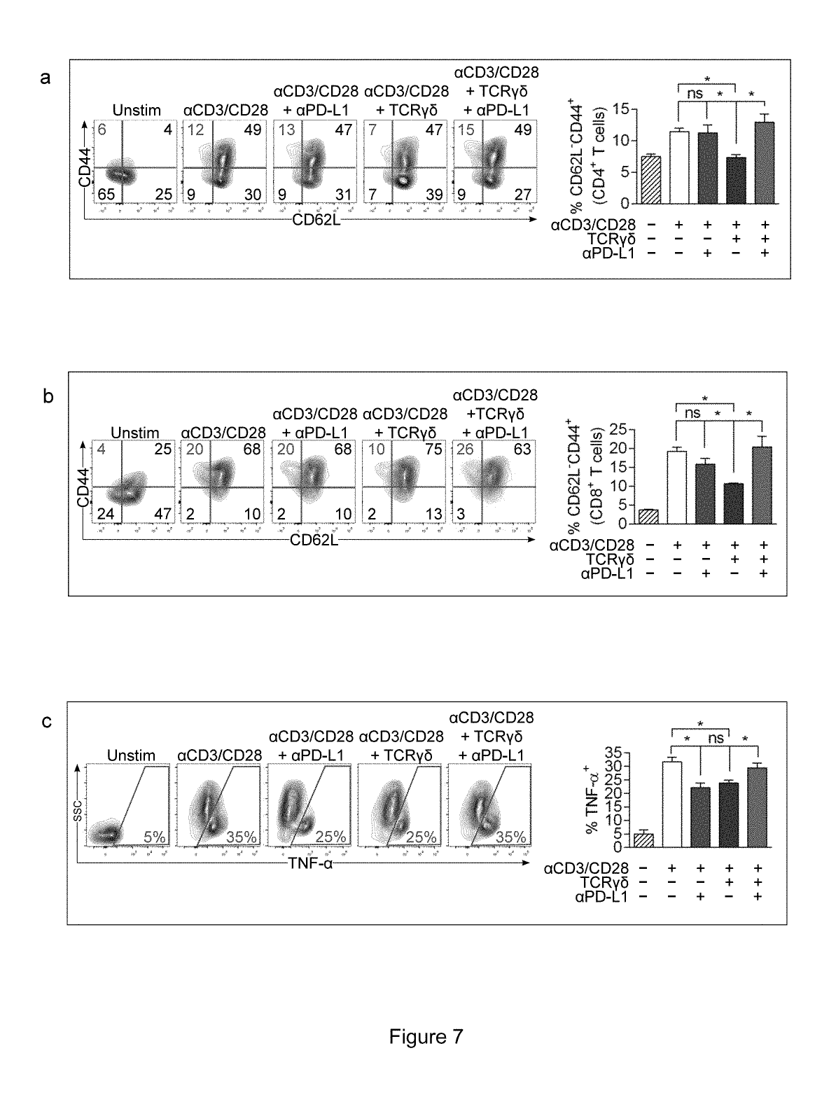

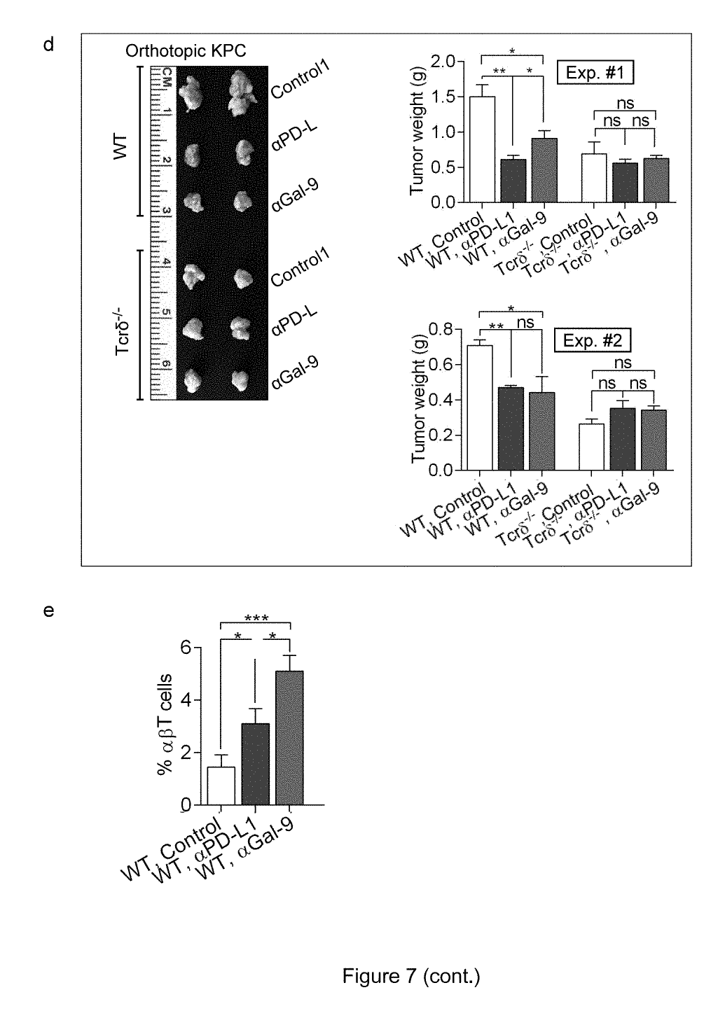

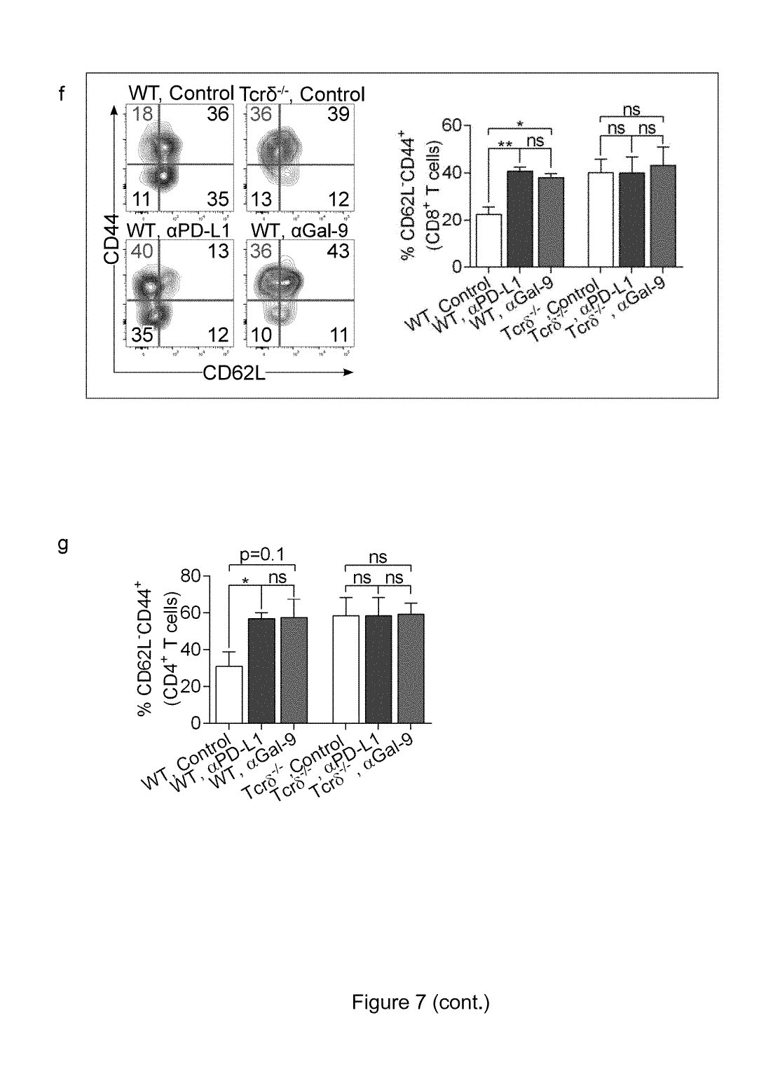

Previous reports have shown that low PD-L1 expression is associated with improved survival in human PDA and that PD-L1 blockade in murine PDA protects mice longitudinally (Nomi et al., 2007, Clinical cancer research: an official journal of the American Association for Cancer Research 13, 2151-2157). We found that Galectin-9 blockade extends animal survival in PDA (FIG. 13f). We postulated that γδT cells promote PDA progression by preventing αβT cell activation via checkpoint receptor ligation. To test this, we again activated spleen CD4⁺ and CD8⁺ T cells in vitro using CD3/CD28 co-ligation alone or in the context of co-culture with PDA-derived γδT cells. Similar to our previous experiments, γδT cells prevented CD4⁺ (FIG. 7a) and CD8⁺ (FIG. 7b) T cells from adopting an activated CD44⁺CD62L⁻ phenotype; however, γδT cell-mediated suppression was reversed with PD-L1 blockade. Further, whereas PDA-infiltrating γδT cells prevented αβT cell expression of TNF-α in vitro, this was again reversed by PD-L1 blockade (FIG. 7c).

To definitively test whether γδT cells promote PDA progression in vivo via checkpoint ligand-dependent immune-suppression, we serially blocked PD-L1 or Galectin-9 using neutralizing mAbs in cohorts of WT and Tcrδ^{-/-} mice challenged with orthotopic KPC-derived tumor. Consistent with our hypothesis, PD-L1 or Galectin-9 blockade protected WT mice but were ineffective at further inducing tumor-protection in Tcrδ^{-/-} animals (FIGS. 7d). Moreover, αPD-L1 and αGalectin-9 each substantially increased αβT cell infiltration of PDA in WT mice (FIG. 7e) but failed to enhance αβT cell infiltration in Tcrδ^{-/-} hosts (not shown). Similarly, both PD-L1 and Galectin-9 blockade in vivo induced an activated CD4⁺ and CD8⁺ T cell phenotype in orthotopic PDA in WT mice but did not enhance αβT cell activation or Th1-polarization in PDA in Tcrδ^{-/-} hosts (FIGS. 7f, g and 13g, h). To determine whether checkpoint ligand antagonism was also only efficacious in γδT cell-competent hosts in a slowly progressive model of PDA, we serially treated cohorts of 6 week old KC;Tcrδ^{+/+} and KC;Tcrδ^{-/-} mice for 8 weeks with an αPD-L1 mAb. Again, PD-L1 inhibition protected KC pancreata from oncogenic progression but offered no benefit in KC;Tcrδ^{-/-} mice (FIG. 13i). Moreover, adoptive transfer of PDA-entrained γδT cells to Tcrδ^{-/-} mice coincident with orthotopic tumor challenge resulted in tumor growth rates comparable to WT mice (FIG. 13j). However, ex-vivo blockade of PD-L1 in γδT cells prior to adoptive transfer failed to accelerate tumor growth (FIG. 13k). To determine whether PDA-infiltrating γδT cells abrogate antigen-restricted anti-tumor immunity in a PD-L1 dependent manner, we directly inoculated PDA-infiltrating γδT cells into established subcutaneous PDA tumors engineered to express OVA in Tcrδ^{-/-} hosts. γδT cell administration again accelerated tumor growth and concomitantly diminished OVA-specific CD8+ T cell proliferation and activation. However, ex-vivo blockade of PD-L1 blockade in γδT cells abrogated their tumor-promoting and immune-suppressive effects (FIG. 13l-m). Collectively, these data imply γδT cells are important mediators of checkpoint receptor dependent immune-suppression in PDA.

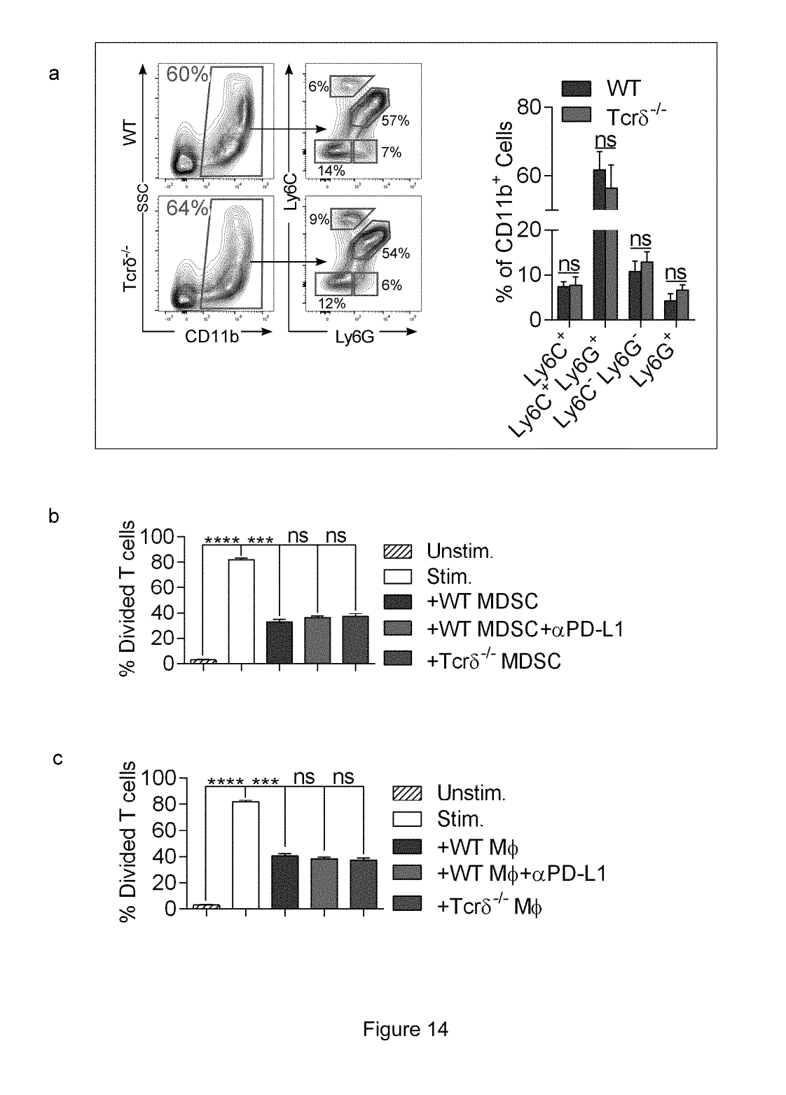

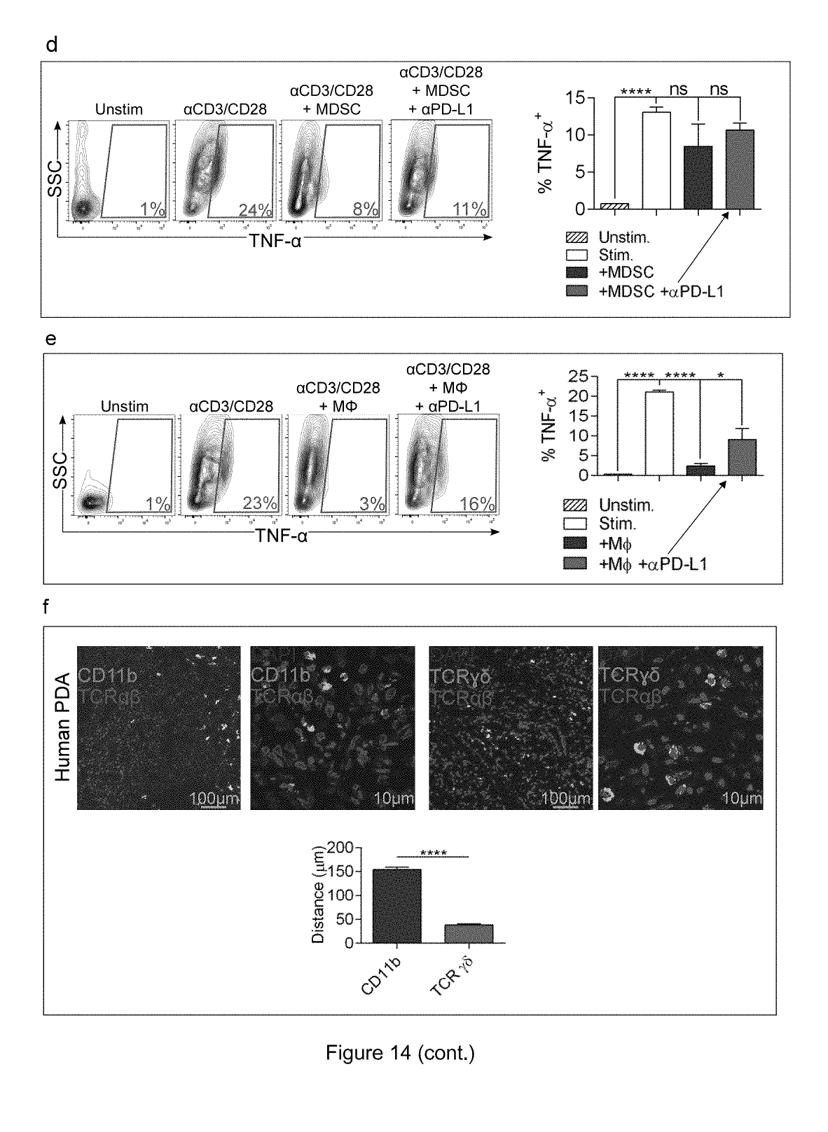

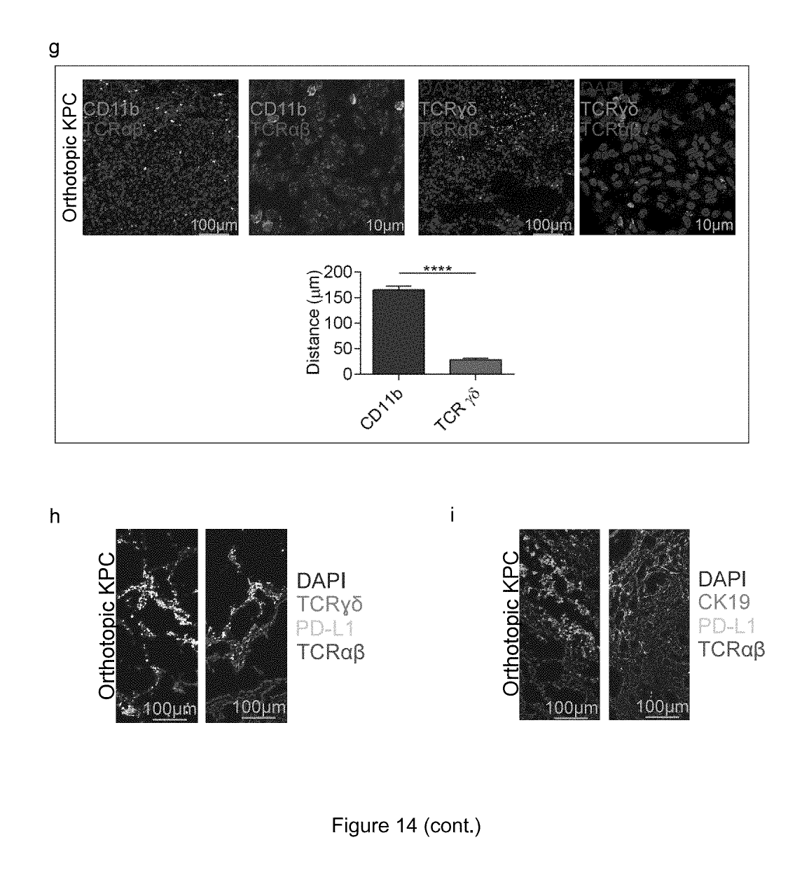

Notably, whereas γδT cell deletion augmented αβT cell infiltration and activation in PDA, it did not alter the fraction of PDA-infiltrating MDSCs or tumor-associated macrophages (TAMs) (FIG. 14a). Similarly, γδT cell deletion did not affect the capacity of MDSCs or TAMs to mitigate T cell proliferation in PDA (FIG. 14b, c). Further, in contrast to the exhaustion ligand-dependent immune-suppressive effects of γδT cells, PDA-infiltrating MDSC inhibition of αβT cell activation was independent of PD-L1 and macrophage-mediated inhibition was only partially mitigated by PD-L1 blockade based on αCD3/αCD28-mediated αβT cell proliferation (FIG. 14b, c), expression of TNF-α (FIG. 14d, e) and adoption of a CD44⁺CD62L⁻ phenotype (not shown). Moreover, whereas αβT cells were in intimate proximity with γδT cells in the PDA TME, myeloid cells were separated by great distances from αβT cells in situ in human PDA (FIG. 14f), in invasive murine PDA (FIG. 14g) and in pre-invasive disease (not shown) suggesting enhanced opportunity for direct γδT cell αβT cell interaction and limited opportunity for direct cross-talk between macrophages and αβT cells. Similarly, whereas αβT cells were in direct contact with PD-L1⁺γδT cells (FIG. 14h), αβT cells were not in close proximity of PD-L1⁺ epithelial cells (FIG. 14i).

### Example 2: Gemcitabine Treatment Increased Suppressive gd T Cells in PDA Mice

C57BL/6 (H-2Kb) and B6.129P2-Tcrd^{tm1Mom}/J (Tcrδ^{-/-}) mice were purchased from Jackson Labs (Bar Harbor, Me.) and bred in-house. Age-matched female mice were used in each experiment. For orthotopic tumor experiments, 8-10 week old mice were used. For orthotopic pancreatic tumor challenge, mice were administered intra-pancreatic injections of tumor cells derived from KPC mice. Cells were suspended in PBS with 50% Matrigel (BD Biosciences, Franklin Lakes, N.J.) and 1×10⁵ tumor cells were injected into the body of the pancreas via laparotomy. Mice were sacrificed 3 weeks later.

To study the effects of gemcitabine hydrochloride, mice were administered gemcitabine hydrochloride (2 mg; Sigma-Aldrich, St. Louis, Mo.) by intraperitoneal (i.p.) injection three times weekly for 3 weeks. In other experiments, animals were treated thrice weekly with i.p. injections of neutralizing mAbs directed against TCR Vγ4 (UC3-10A6, 200 ug; BioXCell, West Lebanon, N.H.). All animal procedures were approved by the New York University School of Medicine IACUC.

Murine single cell suspensions for flow cytometry were prepared as described previously (Daley et al., Cell 166:1485-1499 (2016)). Briefly, pancreata were placed in cold RPMI 1640 with Collagenase IV (1 mg/mL; Worthington Biochemical, Lakewood, N.J.) and DNAse I (2 U/mL; Promega, Madison, Wis.) and minced with scissors to sub-millimeter pieces. Tissues were then incubated at 37° C. for 30 minutes with gentle shaking every 5 minutes. Specimens were passed through a 70 μm mesh, and centrifuged at 350 g for 5 minutes. The cell pellet was resuspended in cold PBS with 1% FBS. Single cell splenocyte suspensions were prepared as previously described (Daley et al., Cell 166:1485-1499 (2016)). Cell labeling was performed after blocking FcγRIII/II with an anti-CD16/CD32 mAb (eBioscience, San Diego, Calif.) by incubating 1×10⁶ cells with 1 μg of fluorescently conjugated mAbs directed against murine CD3 (17A2), CD4 (RM4-5), CD8 (53-6.7), CD45 (30-F11), Tcrγ/δ (GL3), VΓ1 (2.11), Vγ4 (UC3-10A6; all Biolegend, San Diego, Calif.). Flow cytometry was carried out on the LSR-II flow cytometer (BD Biosciences). Data were analyzed using FlowJo v.10.1 (Treestar, Ashland, Oreg.).

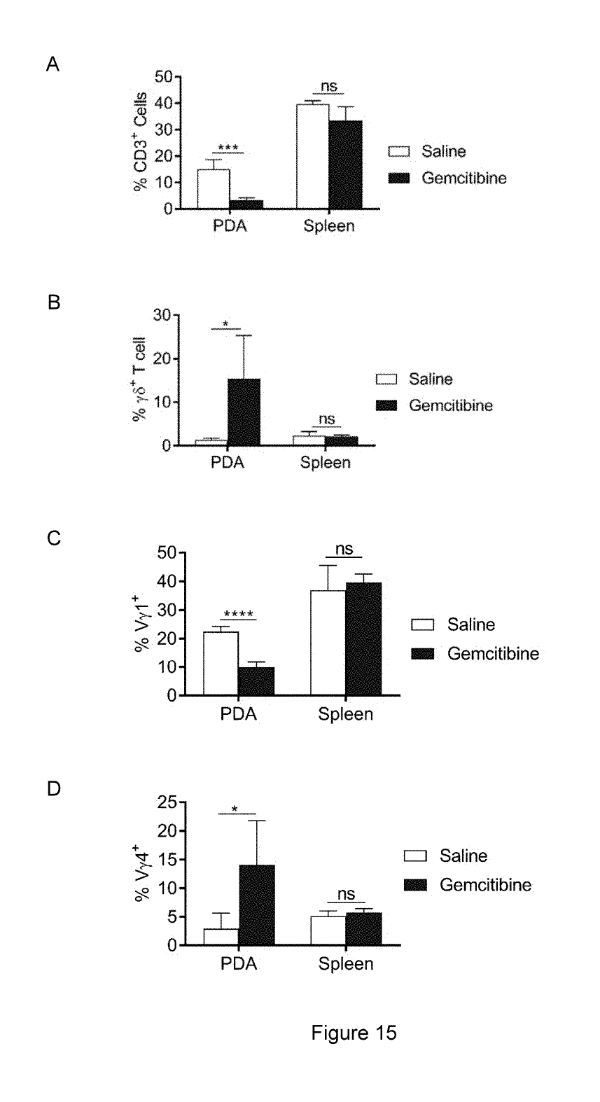

After three weeks of the protocol described above, the number of Vγ1⁺ and Vγ4⁺γδ T cells in the spleen and in the tumor were measured by flow cytometry following the method described above. As shown in FIG. 15, there was a significantly greater percentage of Vγ4⁺γδ T cells in the tumor in mice treated with gemcitibine, and a significantly smaller percentage of Vγ1⁺γδ T cells in the tumor as compared to the tumors of the saline group. Also, there was a significantly greater percentage of γδ T cells in tumor the gemcitibine-treated group compared to the saline group. The difference between groups in the spleen was not significant.

The results from this study indicate that chemotherapeutic agents such as gemcitabine could increase the level of γδ T cells, particularly Vγ4⁺ and/or Vγb 1⁻ cells in TME of PDA mice. Thus, combined PDA therapy of γδ T cell suppressors and chemotherapeutic agents such as gemcitabine would be expected to exert superior therapeutic effects due to, at least, the effect of the γδ T cell suppressors to counteract the enhanced tumor-promoting activity of γδ T cells induced by the chemotherapeutic agents.

### Example 3: γδ T Cell Knockout Mice Showed Lower Tumor Burden in a Colorectal Tumor Mouse Model

C57BL/6 (H-2Kb) and B6.129P2-Tcrd^{tm1Mom}/J (Tcrδ^{-/-}) mice were purchased from Jackson Labs (Bar Harbor, Me.) and bred in-house. Age-matched female mice were used in each experiment. For orthotopic tumor experiments, 8-10 week old mice were used. For orthotopic pancreatic tumor challenge, mice were administered intra-pancreatic injections of tumor cells derived from KPC mice. Cells were suspended in PBS with 50% Matrigel (BD Biosciences, Franklin Lakes, N.J.) and 1×10⁵ tumor cells were injected into the body of the pancreas via laparotomy. Mice were sacrificed 3 weeks later. In select experiments, MC38 cells (1×10⁶ cells in 200 ul Matrigel) were administered subcutaneously and tumor volume was serially recorded using Vernier calipers.

Murine single cell suspensions for flow cytometry were prepared as described previously (Daley et al., Cell 166:1485-1499 (2016)). Briefly, pancreata were placed in cold RPMI 1640 with Collagenase IV (1 mg/mL; Worthington Biochemical, Lakewood, N.J.) and DNAse I (2 U/mL; Promega, Madison, Wis.) and minced with scissors to sub-millimeter pieces. Tissues were then incubated at 37° C. for 30 minutes with gentle shaking every 5 minutes. Specimens were passed through a 70 μm mesh, and centrifuged at 350 g for 5 minutes. The cell pellet was resuspended in cold PBS with 1% FBS. Single cell splenocyte suspensions were prepared as previously described (Daley et al., Cell 166:1485-1499 (2016)). Cell labeling was performed after blocking FcγRIII/II with an anti-CD16/CD32 mAb (eBioscience, San Diego, Calif.) by incubating 1×10⁶ cells with 1 μg of fluorescently conjugated mAbs directed against murine CD3 (17A2), CD4 (RM4-5), CD8 (53-6.7), CD45 (30-F11), Tcrγ/δ (GL3), VΓ1 (2.11), Vγ4 (UC3-10A6; all Biolegend, San Diego, Calif.). Flow cytometry was carried out on the LSR-II flow cytometer (BD Biosciences). Data were analyzed using FlowJo v.10.1 (Treestar, Ashland, Oreg.).

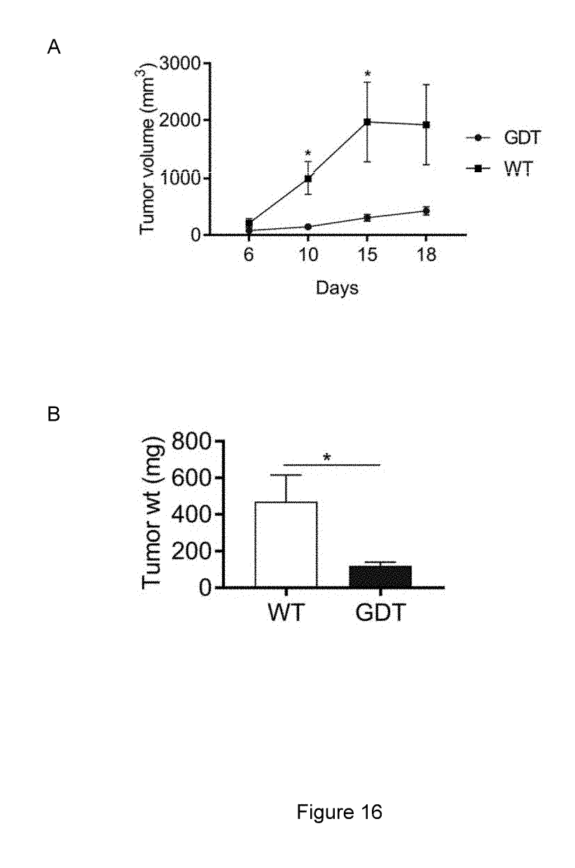

Ten wild-type and 10 γδ T cell knockout mice were administered MCA38 tumor cells (colorectal tumor cells) subcutaneously as described above. The tumor size was measured on days 6, 10, 15, and 18. The weights of the tumors were also measured at the end of the experiment. As shown in FIG. 16, both tumor volume and tumor weight were lower in the γδ T cell knockout mice relative to the control mice.

What is claimed is:

1. A method for treating a solid tumor, comprising administering to a subject in need thereof an effective amount of a γδ T cell suppressor.

2. The method of claim 1, wherein the γδ T cell suppressor is an agent that inhibits an immunosuppressive γδ T cell.

3. The method of claim 2, wherein the immunosuppressive γδ T cell is a circulating γδ T cell or a γδ T cell infiltrated into tumor tissue in the subject.

4. The method of claim 1, wherein the γδ T cell suppressor is an antibody that specifically binds a γδ T cell.

5. The method of claim 4, wherein the antibody is a bi-specific antibody that binds (i) the γδ T cell and (ii) an αβ T cell or NK cell.

6. The method of claim 4, wherein the antibody is a tri-specific antibody that binds (i) they chain of the γδ T cell, (ii) the δ chain of γδ T cell, and (iii) an αβ T cell or NK cell.

7. The method of claim 4, wherein the antibody specifically binds a γδ T cell comprising a δ1 subunit or a δ2 subunit.

8. The method of claim 1, wherein the γδ T cell suppressor is an antibody that blocks recruitment of immunosuppressive γδ T cell to a tumor site in the subject.

9. The method of claim 8, wherein the antibody specifically binds CCR2, CCL2, or CCR6.

10. The method of claim 4, wherein the antibody is a human antibody or a humanized antibody.

11. The method of claim 1, wherein the γδ T cell suppressor is an agent that blocks antigenic expansion of immunosuppressive γδ T cells.

12. The method of claim 1, wherein the γδ T cell suppressor is an immune cell expressing a chimeric receptor that targets immunosuppressive γδ T cells.

13. The method of claim 12, wherein the immune cell is a T cell or an NK cell.

14. The method of claim 1, wherein the subject has undergone another anti-tumor therapy.

15. The method of claim 1, further comprising performing another anti-tumor therapy to the subject.

16. The method of claim 14, wherein the other anti-tumor therapy is chemotherapy, radiotherapy, immunotherapy, therapy involving a small molecule kinase inhibitor, surgery, or a combination thereof.

17. The method of claim 15, wherein the other anti-tumor therapy is chemotherapy, radiotherapy, immunotherapy, therapy involving a small molecule kinase inhibitor, surgery, or a combination thereof.

18. The method of claim 15, wherein the performing step comprises administering to the subject an inhibitor of a checkpoint molecule, an agonist of a co-stimulatory receptor, or an inhibitor of an innate immune cell target.

19. The method of claim 18, wherein the checkpoint molecule is selected from the group consisting of PD-1, PD-L1, PD-L2, CTLA-4, LAG3, TIM-3 and A2aR.

20. The method of claim 18, wherein the co-stimulatory receptor is selected from the group consisting of OX40, GITR, CD137, CD40, CD27, and ICOS.

21. The method of claim 18, wherein the innate immune cell target is selected from the group consisting of KIR, NKG2A, CD96, TLR, and IDO.

22. The method of claim 11, wherein the subject is administered an inhibitor of a checkpoint molecule, which is an anti-PD-L1 antibody.

23. The method of claim 15, wherein the performing step comprises administering to the subject a chemotherapeutic agent.

24. The method of claim 23, wherein the chemotherapeutic agent is gemcitabine or abraxane.

25. The method of claim 1, wherein the subject is a human patient having the solid tumor.

26. The method of claim 25, wherein the solid tumor is pancreatic duct adenocarcinoma (PDA), colorectal cancer (CRC), melanoma, breast cancer, lung cancer (for example, non-small cell lung cancer, NSCLC, and small cell lung cancer, SCLC), upper and lower gastrointestinal malignancies (including, but not limited to, esophageal, gastric, and hepatobiliary cancer), squamous cell head and neck cancer, genitourinary cancers, and sarcomas.

27. A kit for treating a solid tumor in a subject, the kit comprising:
(i) a first pharmaceutical composition that comprises a γδ T cell suppressor, and
(ii) a second pharmaceutical composition that comprises a chemotherapeutic agent, an inhibitor of a checkpoint molecule, an agonist of a co-stimulatory receptor, or an inhibitor of an innate immune cell target.

28. A pharmaceutical composition, comprising (i) a γδ T cell suppressor, and (ii) an inhibitor of a checkpoint molecule, an agonist of a co-stimulatory receptor, or an inhibitor of an innate immune cell target.

29. A method for analyzing a sample, the method comprising:
(i) obtaining a biological sample from a subject suspected of having pancreatic ductal adenocarcinoma (PDA) or colorectal cancer (CRC); and
(ii) measuring the level of γδ T cells in the biological sample.

30. The method of claim 29, wherein the γδ T cells are effector memory γδ T (TEM) cells.

31. The method of claim 29, wherein the biological sample is a peripheral blood sample.

32. The method of claim 29, wherein the biological sample is a tissue sample obtained from a suspected tumor site.

33. The method of claim 29, wherein the measuring step involves an antibody that specifically binds γδ T cells.

34. The method of claim 33, wherein the antibody specifically binds γδ T cells expressing a T cell receptor comprising a δ1 subunit or a δ2 subunit.

35. The method of claim 29, further comprising measuring the level of a checkpoint molecule, the level of Galectin-9, or both in the biological sample.

36. The method of claim 35, wherein the checkpoint molecule is PD-L1.

37. The method of claim 29, further comprising identifying the subject as having or at risk for PDA or CRC based on the level of the γδ T cells in the biological sample determined in (ii), wherein an elevated level of γδ T cells relative to that of a control subject is indicative of presence or risk of PDA or CRC.

38. The method of claim 37, further comprising performing a treatment of PDA or CRC to the subject, if the subject is identified as having or at risk for PDA or CRC.

39. The method of claim 29, wherein the subject is a human subject.
